(19) United States
(12) Patent Application Publication
(10) Pub. No.: US 2023/0082497 A1
(43) Pub. Date: Mar. 16, 2023

(54) GENETICALLY ENGINEERED PLANTS THAT EXPRESS 6-PHOSPHOGLUCONATE DEHYDRATASE AND/OR 2-KETO-3-DEOXY-6-PHOSPHOGLUCONATE ALDOLASE

(71) Applicant: YIELD10 BIOSCIENCE, INC., Woburn, MA (US)

(72) Inventors: Frank Anthony SKRALY, Woburn, MA (US); Jihong TANG, Woburn, MA (US); Kristi D. SNELL, Belmont, MA (US)

(21) Appl. No.: 17/904,343

(22) PCT Filed: Feb. 19, 2021
(86) PCT No.: PCT/US2021/018743

§ 371 Date: Aug. 16, 2022

Related U.S. Application Data
(60) Provisional application No. 62/979,198, filed on Feb. 20, 2020.

(51) Int. Cl. C12N 15/82; C12N 9/88

(52) U.S. Cl. CPC C12N 15/8245; C12N 15/8261; C12N 9/88; C12Y 401/02014; C12Y 402/01012

(57) ABSTRACT

A genetically engineered plant that expresses a 6-phosphogluconate dehydratase (EDD) and/or a 2-keto-3-deoxy-6-phosphogluconate aldolase (EDA) is provided. The plant comprises at least one of a first or second modified gene. The first modified gene comprises a first promoter and a nucleic acid sequence encoding the EDD. The first promoter is non-cognate with respect to the nucleic acid sequence encoding the EDD. The first modified gene is configured so transcription of the nucleic acid sequence encoding the EDD is initiated from the first promoter and results in expression of the EDD. The second modified gene comprises a second promoter and a nucleic acid sequence encoding the EDA. The second promoter is non-cognate with respect to the nucleic acid sequence encoding the EDA. The second modified gene is configured so transcription of the nucleic acid sequence encoding the EDA is initiated from the second promoter and results in expression of the EDA.

### FIELD OF THE INVENTION

The present invention relates generally to genetically engineered plants that express a 6-phosphogluconate dehydratase (also termed EDD) and/or a 2-keto-3-deoxy-6-phosphogluconate aldolase (also termed EDA), and more particularly to such genetically engineered plants comprising at least one of a first modified gene or a second modified gene, wherein the first modified gene comprises (i) a first promoter and (ii) a nucleic acid sequence encoding the EDD, the first promoter is non-cognate with respect to the nucleic acid sequence encoding the EDD, the first modified gene is configured such that transcription of the nucleic acid sequence encoding the EDD is initiated from the first promoter and results in expression of the EDD, the second modified gene comprises (i) a second promoter and (ii) a nucleic acid sequence encoding the EDA, the second promoter is non-cognate with respect to the nucleic acid sequence encoding the EDA, and the second modified gene is configured such that transcription of the nucleic acid sequence encoding the EDA is initiated from the second promoter and results in expression of the EDA.

### BACKGROUND OF THE INVENTION

The world faces a major challenge in the next 35 years to meet the increased demands for food production to feed a growing global population, which is expected to reach 9 billion by the year 2050. Food output will need to be increased by up to 70% in view of the growing population, increased demand for improved diet, land use changes for new infrastructure, alternative uses for crops and changing weather patterns due to climate change. Studies have shown that traditional crop breeding alone will not be able to solve this problem (Deepak K. Ray, Nathaniel D. Mueller, Paul C. West and Jonathon A. Foley, 2013. Yield trends are Insufficient to Double Global Crop Production by 2050. PLOS, published Jun. 19, 2013 doi.org/10.1371/journal.pone.0066428). There is therefore a need to develop new technologies to enable step change improvements in crop performance and in particular crop productivity and/or yield.

Major agricultural crops include food crops, such as maize, wheat, oats, barley, soybean, millet, sorghum, pulses, bean, tomato, corn, rice, cassava, sugar beets, and potatoes, forage crop plants, such as hay, alfalfa, and silage corn, and oilseed crops, such as Camelina sativa, Brassica species (e.g. B. napus (canola), B. rapa, B. juncea, and B. carinata), crambe, soybean, sunflower, safflower, oil palm, flax, and cotton, among others. Productivity of these crops, and others, is limited by numerous factors, including for example relative inefficiency of photochemical conversion of light energy to fixed carbon during photosynthesis, as well as loss of fixed carbon by photorespiration and/or other essential metabolic pathways having enzymes catalyzing decarboxylation reactions. For seed (grain), tuber or fruit crops, the ratio of seed, tubers or fruit produced per unit plant biomass, which is referred to as the harvest index, is a major determinant of crop productivity.

Increasing seed, fruit or tuber yield in major crops can be viewed as a two-step carbon optimization problem; the first is improving photosynthetic carbon fixation and the second is optimizing the flow of fixed carbon to seed production and/or conversion of the carbon within the seed, fruit or tuber versus vegetative biomass (roots, stems, leaves etc.). There is a particular need to develop new technologies to enable step change improvements in the harvest index of seed, fruit and tuber crops.

It is an objective of this invention to provide genes, systems and plants that provide improved carbon conversion efficiency (also termed “CCE,” corresponding to moles of carbon in biomass per mole of carbon in phloem-supplied substrates) in seeds by expressing genes encoding proteins with 6-phosphogluconate dehydratase (EDD; EC 4.2.1.12) and/or 2-keto-3-deoxy-6-phosphogluconate aldolase (EDA; EC 4.1.2.14) activity. In a preferred embodiment the expressed EDD and EDA proteins are operably linked to a peptide signal such that the expressed EDD and EDA proteins are targeted to the plastid of the plant cells. It is expected that plants which have been engineered to have the higher levels of EDD and EDA expression in the plastid have increased carbon conversion efficiency and/or higher seed yield than the same plant which has not been engineered to increase EDD and EDA expression.

### BRIEF SUMMARY OF THE INVENTION

Methods, genes and systems for producing plant cells, tissues, and plants expressing 6-phosphogluconate dehydratase (EDD; EC 4.2.1.12) and/or 2-keto-3-deoxy-6-phosphogluconate aldolase (EDA; EC 4.1.2.14) are disclosed. The plant cells, tissues, and plants preferably express both EDD and EDA, preferably such that expression of one or both are modulated and/or increased. The plant cells, tissues, and plants are made by genetic engineering by introducing at least one modified gene encoding at least one of EDD and/or EDA. For plant cells, tissues, and plants in which a modified gene encoding EDD and a modified gene encoding EDA have been introduced, the EDD and the EDA can be expressed from the modified genes. For plant cells, tissues, and plants in which a modified gene encoding EDD has been introduced, but no modified gene encoding EDA has been introduced, the EDA may be encoded by an endogenous gene, such that the EDD is expressed from the modified gene and the EDA is expressed from the endogenous gene. Similarly, for plant cells, tissues, and plants in which a modified gene encoding EDA has been introduced, but no modified gene encoding EDD has been introduced, the EDD may be encoded by an endogenous gene, such that the EDA is expressed from the modified gene and the EDD is expressed from the endogenous gene. The plant cells, tissues, and plants can exhibit increased expression of EDD and/or EDA in the plastid such that the carbon conversion efficiency (CCE; moles of carbon in biomass per mole of carbon in phloem-supplied substrates) is increased, resulting in increased crop performance and/or yield. The genes encoding the EDD and/or EDA can be used alone or in combination with altered expression of additional genes to enhance photosynthesis or carbon partitioning to seed. The expression of the genes encoding the EDD and/or EDA proteins can be increased using genetic engineering techniques to develop plants with increased performance and/or yield. Where genetic engineering techniques are used to increase the expression of the EDD and/or EDA proteins, the increased expression can be accomplished using transgenic technologies with EDD and/or EDA genes from a source other than the plant being modified, or by genome editing approaches to increase the expression of the EDD and/or EDA genes in constitutive, seed-specific, and/or seed-preferred manners.

Thus, a genetically engineered plant that expresses a 6-phosphogluconate dehydratase (EDD) and/or a 2-keto-3-deoxy-6-phosphogluconate aldolase (EDA) is provided. The genetically engineered plant comprises at least one of a first modified gene or a second modified gene. The first modified gene comprises (i) a first promoter and (ii) a nucleic acid sequence encoding the EDD. The first promoter is non-cognate with respect to the nucleic acid sequence encoding the EDD. The first modified gene is configured such that transcription of the nucleic acid sequence encoding the EDD is initiated from the first promoter and results in expression of the EDD. The second modified gene comprises (i) a second promoter and (ii) a nucleic acid sequence encoding the EDA. The second promoter is non-cognate with respect to the nucleic acid sequence encoding the EDA. The second modified gene is configured such that transcription of the nucleic acid sequence encoding the EDA is initiated from the second promoter and results in expression of the EDA.

In some embodiments, the EDD is characterized as EC 4.2.1.12. In some embodiments, the EDD converts 6-phosphogluconate (6PG) to 2-keto-3-deoxy phosphogluconate (KDPG) and water. In some embodiments, the EDD is one or more of a bacterial EDD, a cyanobacterial EDD, an algal EDD, or a plant EDD. In some embodiments, the EDD has at least 30% or higher sequence identity to one or more of the following: (1) Zymomonas mobilis EDD of SEQ ID NO: 44; (2) Synechocystis sp. PCC 6803 EDD of SEQ ID NO: 136; (3) Guillardia theta EDD of SEQ ID NO: 140; or (4) Hordeum vulgare EDD of SEQ ID NO: 139. In some embodiments, the EDD comprises one or more of the following: (1) Zymomonas mobilis EDD of SEQ ID NO: 44; (2) Synechocystis sp. PCC 6803 EDD of SEQ ID NO: 136; (3) Guillardia theta EDD of SEQ ID NO: 140; or (4) Hordeum vulgare EDD of SEQ ID NO: 139.

In some embodiments, the EDA is characterized as EC 4.1.2.14. In some embodiments, the EDA converts 2-keto-3-deoxy-6-phosphogluconate (KDPG) to pyruvate and D-glyceraldehyde 3-phosphate. In some embodiments, the EDA is one or more of a bacterial EDA, a cyanobacterial EDA, an algal EDA, or a plant EDA. In some embodiments, the EDA has at least 30% or higher sequence identity to one or more of the following: (1) Zymomonas mobilis EDA of SEQ ID NO: 70; (2) Synechocystis sp. PCC 6803 EDA of SEQ ID NO: 137; (3) Phaeodactylum tricornutum EDA of SEQ ID NO: 92; or (4) Hordeum vulgare EDA of SEQ ID NO: 97. In some embodiments, the EDA comprises one or more of the following: (1) Zymomonas mobilis EDA of SEQ ID NO: 70; (2) Synechocystis sp. PCC 6803 EDA of SEQ ID NO: 137; (3) Phaeodactylum tricornutum EDA of SEQ ID NO: 92; or (4) Hordeum vulgare EDA of SEQ ID NO: 97.

In some embodiments, the genetically engineered plant expresses both the EDD and the EDA. In some of the embodiments, the genetically engineered plant comprises both the first modified gene and the second modified gene. In others of these embodiments, the genetically engineered plant comprises the first modified gene, lacks the second modified gene, and further comprises an endogenous gene encoding the EDA. In still others of these embodiments, the genetically engineered plant lacks the first modified gene, comprises the second modified gene, and further comprises an endogenous gene encoding the EDD.

In some embodiments, the first promoter comprises one or more of a constitutive promoter, a seed-specific promoter, or a seed-preferred promoter.

In some embodiments, the second promoter comprises one or more of a constitutive promoter, a seed-specific promoter, or a seed-preferred promoter.

In some embodiments, the genetically engineered plant exhibits modulated expression of the EDD and the EDA relative to a reference plant that does not comprise the at least one of the first modified gene or the second modified gene.

In some embodiments, the genetically engineered plant exhibits increased expression of the EDD and the EDA relative to a reference plant that does not comprise the at least one of the first modified gene or the second modified gene.

In some embodiments, the genetically engineered plant exhibits increased expression of the EDD and the EDA in plastids of cells of the genetically engineered plant relative to a reference plant that does not comprise the at least one of the first modified gene or the second modified gene.

In some embodiments, the first modified gene further comprises a nucleic acid sequence encoding a plastid targeting sequence and is further configured such that the EDD comprises an N-terminal plastid targeting signal, and the second modified gene further comprises a nucleic acid sequence encoding a plastid targeting sequence and is further configured such that the EDA comprises an N-terminal plastid targeting signal.

In some embodiments, the genetically engineered plant further comprises one or more additional modified genes, each of the one or more additional modified genes comprising (i) a respective promoter and (ii) a respective nucleic acid sequence encoding one or more of glucose-6-phosphate dehydrogenase (ZWF), 6-phosphogluconolactonase (PGL), glucose dehydrogenase (GDH), or gluconate kinase (GCK), each respective promoter being non-cognate with respect to its respective nucleic acid sequence encoding the one or more of ZWF, PGL, GDH, or GCK, and each additional modified gene being configured such that transcription of its respective nucleic acid sequence encoding the one or more of ZWF, PGL, GDH, or GCK is initiated from its respective promoter and results in expression of the one or more of ZWF, PGL, GDH, or GCK.

In some embodiments, the genetically engineered plant has a carbon conversion efficiency that is at least 5% higher, at least 10% higher, at least 20% higher, at least 40% higher, at least 80% higher, at least 120% higher, or at least 160% higher than for a reference plant that does not comprise the at least one of the first modified gene or the second modified gene.

In some embodiments, the genetically engineered plant has an increased sink strength in comparison to a reference plant that does not comprise the at least one of the first modified gene or the second modified gene.

In some embodiments, the genetically engineered plant has one or more characteristics selected from higher performance and/or seed, fruit or tuber yield relative to a reference plant that does not comprise the at least one of the first modified gene or the second modified gene. In some of these embodiments, the one or more characteristics are increased by 10%, 20%, 30%, 40%, 50%, 60%, 70%, 80%, 90%, 100% or higher relative to a reference plant that does not comprise the at least one of the first modified gene or the second modified gene.

In some embodiments, the genetically engineered plant comprises one or more of Camelina sativa, camelina species, Brassica species, Brassica napus (canola), Brassica rapa, Brassica juncea, Brassica carinata, crambe, soybean, sunflower, safflower, oil palm, flax, or cotton. In some embodiments, the genetically engineered plant comprises one or more of maize, wheat, oat, barley, soybean, Brassica species, Brassica napus (canola), rapeseed, Brassica rapa, Brassica carinata, Brassica juncea, Thlaspi caerulescens (pennycress), sunflower, safflower, oil palm, millet, sorghum, potato, lentil, chickpea, pea, pulse, bean, tomato, potato, or rice.

A method for producing the genetically engineered plant also is provided. The method comprises a step of: (1) introducing at least one of the first modified gene or the second modified gene into a plant, thereby obtaining the genetically engineered plant.

In some embodiments, the step (1) comprises transforming the plant cell of the plant with the at least one of the first modified gene or the second modified gene. In some embodiments, the step (1) comprises transforming the plant cell of the plant with both the first modified gene and the second modified gene.

In some embodiments, the method further comprising steps of: (2) selecting the transformed plant cell on a selective medium; (3) regenerating the selected transformed plant cell to produce a differentiated plant; and (4) selecting the differentiated plant based on expression of the at least one of the first modified gene or the second modified gene in at least one of a tissue or a cellular location of the differentiated plant, thereby obtaining the genetically engineered plant.

Exemplary embodiments include the following:

Embodiment 1. A genetically engineered plant that expresses a 6-phosphogluconate dehydratase (EDD) and/or a 2-keto-3-deoxy-6-phosphogluconate aldolase (EDA), the genetically engineered plant comprising at least one of a first modified gene or a second modified gene, wherein:

the first modified gene comprises (i) a first promoter and (ii) a nucleic acid sequence encoding the EDD;

the first promoter is non-cognate with respect to the nucleic acid sequence encoding the EDD;

the first modified gene is configured such that transcription of the nucleic acid sequence encoding the EDD is initiated from the first promoter and results in expression of the EDD;

the second modified gene comprises (i) a second promoter and (ii) a nucleic acid sequence encoding the EDA;

the second promoter is non-cognate with respect to the nucleic acid sequence encoding the EDA; and

the second modified gene is configured such that transcription of the nucleic acid sequence encoding the EDA is initiated from the second promoter and results in expression of the EDA.

Embodiment 2. The genetically engineered plant according to embodiment 1, wherein the EDD is characterized as EC 4.2.1.12.

Embodiment 3. The genetically engineered plant according to embodiment 1 or 2, wherein the EDD converts 6-phosphogluconate (6PG) to 2-keto-3-deoxy-6-phosphogluconate (KDPG) and water.

Embodiment 4. The genetically engineered plant according to any one of embodiments 1-3, wherein the EDD is one or more of a bacterial EDD, a cyanobacterial EDD, an algal EDD, or a plant EDD.

Embodiment 5. The genetically engineered plant according to any one of embodiments 1-4, wherein the EDD has at least 30% or higher sequence identity to one or more of the following:

(1) Zymomonas mobilis EDD of SEQ ID NO: 44;

(2) Synechocystis sp. PCC 6803 EDD of SEQ ID NO: 136;

(3) Guillardia theta EDD of SEQ ID NO: 140; or

(4) Hordeum vulgare EDD of SEQ ID NO: 139.

Embodiment 6. The genetically engineered plant according to any one of embodiments 1-5, wherein the EDD comprises one or more of the following:

(1) Zymomonas mobilis EDD of SEQ ID NO: 44;

(2) Synechocystis sp. PCC 6803 EDD of SEQ ID NO: 136;

(3) Guillardia theta EDD of SEQ ID NO: 140; or

(4) Hordeum vulgare EDD of SEQ ID NO: 139.

Embodiment 7. The genetically engineered plant according to any one of embodiments 1-6, wherein the EDA is characterized as EC 4.1.2.14.

Embodiment 8. The genetically engineered plant according to any one of embodiments 1-7, wherein the EDA converts 2-keto-3-deoxy-6-phosphogluconate (KDPG) to pyruvate and D-glyceraldehyde 3-phosphate.

Embodiment 9. The genetically engineered plant according to any one of embodiments 1-8, wherein the EDA is one or more of a bacterial EDA, a cyanobacterial EDA, an algal EDA, or a plant EDA.

Embodiment 10. The genetically engineered plant according to any one of embodiments 1-9, wherein the EDA has at least 30% or higher sequence identity to one or more of the following:

(1) Zymomonas mobilis EDA of SEQ ID NO: 70;

(2) Synechocystis sp. PCC 6803 EDA of SEQ ID NO: 137;

(3) Phaeodactylum tricornutum EDA of SEQ ID NO: 92; or

(4) Hordeum vulgare EDA of SEQ ID NO: 97.

Embodiment 11. The genetically engineered plant according to any one of embodiments 1-10, wherein the EDA comprises one or more of the following:

(1) Zymomonas mobilis EDA of SEQ ID NO: 70;

(2) Synechocystis sp. PCC 6803 EDA of SEQ ID NO: 137;

(3) Phaeodactylum tricornutum EDA of SEQ ID NO: 92; or

(4) Hordeum vulgare EDA of SEQ ID NO: 97.

Embodiment 12. The genetically engineered plant according to any one of embodiments 1-11, wherein the genetically engineered plant expresses both the EDD and the EDA.

Embodiment 13. The genetically engineered plant according to embodiment 12, wherein the genetically engineered plant comprises both the first modified gene and the second modified gene.

Embodiment 14. The genetically engineered plant according to embodiment 12, wherein the genetically engineered plant comprises the first modified gene, lacks the second modified gene, and further comprises an endogenous gene encoding the EDA.

Embodiment 15. The genetically engineered plant according to embodiment 12, wherein the genetically engineered plant lacks the first modified gene, comprises the second modified gene, and further comprises an endogenous gene encoding the EDD.

Embodiment 16. The genetically engineered plant according to any one of embodiments 1-15, wherein the first promoter comprises one or more of a constitutive promoter, a seed-specific promoter, or a seed-preferred promoter.

Embodiment 17. The genetically engineered plant according to any one of embodiments 1-16, wherein the second promoter comprises one or more of a constitutive promoter, a seed-specific promoter, or a seed-preferred promoter.

Embodiment 18. The genetically engineered plant according to any one of embodiments 1-17, wherein the genetically engineered plant exhibits modulated expression of the EDD and the EDA relative to a reference plant that does not comprise the at least one of the first modified gene or the second modified gene.

Embodiment 19. The genetically engineered plant according to any one of embodiments 1-18, wherein the genetically engineered plant exhibits increased expression of the EDD and the EDA relative to a reference plant that does not comprise the at least one of the first modified gene or the second modified gene.

Embodiment 20. The genetically engineered plant according to any one of embodiments 1-19, wherein the genetically engineered plant exhibits increased expression of the EDD and the EDA in plastids of cells of the genetically engineered plant relative to a reference plant that does not comprise the at least one of the first modified gene or the second modified gene.

Embodiment 21. The genetically engineered plant according to any one of embodiments 1-20, wherein the first modified gene further comprises a nucleic acid sequence encoding a plastid targeting sequence and is further configured such that the EDD comprises an N-terminal plastid targeting signal, and the second modified gene further comprises a nucleic acid sequence encoding a plastid targeting sequence and is further configured such that the EDA comprises an N-terminal plastid targeting signal.

Embodiment 22. The genetically engineered plant according to any one of embodiments 1-21, wherein the genetically engineered plant further comprises one or more additional modified genes,

each of the one or more additional modified genes comprising (i) a respective promoter and (ii) a respective nucleic acid sequence encoding one or more of glucose phosphate dehydrogenase (ZWF), 6-phosphogluconolactonase (PGL), glucose dehydrogenase (GDH), or gluconate kinase (GCK);

each respective promoter being non-cognate with respect to its respective nucleic acid sequence encoding the one or more of ZWF, PGL, GDH, or GCK; and

each additional modified gene being configured such that transcription of its respective nucleic acid sequence encoding the one or more of ZWF, PGL, GDH, or GCK is initiated from its respective promoter and results in expression of the one or more of ZWF, PGL, GDH, or GCK.

Embodiment 23. The genetically engineered plant according to any one of embodiments 1-22, wherein the genetically engineered plant has a carbon conversion efficiency that is at least 5% higher, at least 10% higher, at least 20% higher, at least 40% higher, at least 80% higher, at least 120% higher, or at least 160% higher than for a reference plant that does not comprise the at least one of the first modified gene or the second modified gene.

Embodiment 24. The genetically engineered plant according to any one of embodiments 1-23, wherein the genetically engineered plant has an increased sink strength in comparison to a reference plant that does not comprise the at least one of the first modified gene or the second modified gene.

Embodiment 25. The genetically engineered plant according to any one of embodiments 1-24, wherein the genetically engineered plant has one or more characteristics selected from higher performance and/or seed, fruit or tuber yield relative to a reference plant that does not comprise the at least one of the first modified gene or the second modified gene.

Embodiment 26. The genetically engineered plant according to embodiment 25, wherein the one or more characteristics are increased by 10%, 20%, 30%, 40%, 50%, 60%, 70%, 80%, 90%, 100% or higher relative to a reference plant that does not comprise the at least one of the first modified gene or the second modified gene.

Embodiment 27. The genetically engineered plant according to any one of embodiments 1-26, wherein the genetically engineered plant comprises one or more of Camelina sativa, camelina species, Brassica species, Brassica napus (canola), Brassica rapa, Brassica juncea, Brassica carinata, crambe, soybean, sunflower, safflower, oil palm, flax, or cotton.

Embodiment 28. The genetically engineered plant according to any one of embodiments 1-27, wherein the genetically engineered plant comprises one or more of maize, wheat, oat, barley, soybean, Brassica species, Brassica napus (canola), rapeseed, Brassica rapa, Brassica carinata, Brassica juncea, Thlaspi caerulescens (pennycress), sunflower, safflower, oil palm, millet, sorghum, potato, lentil, chickpea, pea, pulse, bean, tomato, or rice.

Embodiment 29. A method for producing the genetically engineered plant of any one of embodiments 1-28, the method comprising a step of:

(1) introducing at least one of the first modified gene or the second modified gene into a plant, thereby obtaining the genetically engineered plant.

Embodiment 30. The method according to embodiment 29, wherein the step (1) comprises transforming the plant cell of the plant with the at least one of the first modified gene or the second modified gene.

Embodiment 31. The method according to embodiment 29, wherein the step (1) comprises transforming the plant cell of the plant with both the first modified gene and the second modified gene.

Embodiment 32. The method according to embodiment 30 or 31, the method further comprising steps of:

(2) selecting the transformed plant cell on a selective medium;

(3) regenerating the selected transformed plant cell to produce a differentiated plant; and

(4) selecting the differentiated plant based on expression of the at least one of the first modified gene or the second modified gene in at least one of a tissue or a cellular location of the differentiated plant, thereby obtaining the genetically engineered plant.

### BRIEF DESCRIPTION OF THE DRAWINGS

FIG. 1 depicts pathways of sugar catabolism in the seed showing the Entner-Doudoroff pathway, the oxidative pentose phosphate pathway, and glycolysis (Embden-Meyerhof-Parnas pathway). Enzyme abbreviations: GDH, glucose dehydrogenase; GCK, gluconate kinase; ZWF, glucose-6-phosphate dehydrogenase; PGL, 6-phosphogluconolactonase; HXK, hexokinase; GPI, glucose-6-phosphate isomerase; PFK, phosphofructokinase; EDD, 6-phosphogluconate dehydratase; EDA, 2-keto-3-deoxy-6-phosphogluconate (KDPG) aldolase; GAPDH, glyceraldehyde-3-phosphate dehydrogenase; TAL, transaldolase; TKT, transketolase; TPI, triose phosphate isomerase; PGK, phosphoglycerate kinase; PGM, phosphoglycerate mutase; Eno, enolase; PK, pyruvate kinase; RPE, ribulose-phosphate 3-epimerase; RPI, ribose-5-phosphate isomerase; GND, 6-phosphogluconate dehydrogenase. Compound abbreviations: KDPG, 2-Keto-3-deoxy phosphogluconate; G6P, glucose-6-phosphate; 6PG, 6-phosphogluconate; F6P, fructose phosphate; F16BP, fructose-1,6-bisphosphate; DHAP, dihydroxyacetone phosphate; GAP, glyceraldehyde-3-phosphate; 3-PGA, 3-phosphoglycerate; E4P, erythrose-4-phosphate; S7P, sedoheptulose-7-phosphate; R5P, ribose-5-phosphate; Ru5P, ribulose-5-phosphate; Xu5P, xylulose-5-phosphate.

FIG. 2 depicts the use of Rubisco without the Calvin cycle in the seed to capture CO₂. The 3-PGA produced is converted to pyruvate using PGM, Eno, and PK activities. Abbreviations are as follows: PRK, phosphoribulokinase; Ru15BP, ribulose-1,5-bisphosphate. All other abbreviations are as in FIG. 1.

FIG. 3 shows a map for pED01 (SEQ ID NO: 152), a transformation vector designed for Agrobacterium-mediated transformation of dicots to express EDD and EDA genes from Zymomonas mobilis. The vector contains the soybean oleosin promoter (SEQ ID NO: 11), operably linked to the signal peptide coding sequence of the small subunit of Rubisco from pea (Cashmore, “Nuclear genes encoding the small subunit of ribulose-1,5-bisphosphate carboxylase” in Genetic Engineering of Plants, T Kosuge, Meredith, C. P. & Hollaender, A., Ed. (Plenum, N.Y., 1983), pp. 29-38), operably linked to a gene encoding EDD from Zymomonas mobilis protein (SEQ ID NO: 44), operably linked to a termination sequence from the soybean oleosin gene. A second expression cassette contains the soybean oleosin promoter, operably linked to the signal peptide coding sequence of the small subunit of Rubisco from pea, operably linked to a gene encoding EDA from Zymomonas mobilis protein (SEQ ID NO: 70), operably linked to a termination sequence from the soybean oleosin gene. A separate expression cassette for the visual marker DsRed2B is used to identify transgenic seeds.

FIG. 4 shows a map for pED02 (SEQ ID NO: 153), a transformation vector designed for Agrobacterium-mediated transformation of dicots to express EDD and EDA genes from the cyanobacteria Synechocystis sp. PCC 6803. The vector contains the soybean oleosin promoter (SEQ ID NO: 11), operably linked to the signal peptide coding sequence of the small subunit of Rubisco from pea (Cashmore, 1983), operably linked to a gene encoding EDD from Synechocystis sp. PCC 6803 (SEQ ID NO: 136), operably linked to a termination sequence from the soybean oleosin gene. A second expression cassette contains the soybean oleosin promoter, operably linked to the signal peptide coding sequence of the small subunit of Rubisco from pea, operably linked to a gene encoding EDA from Synechocystis sp. PCC 6803 protein (SEQ ID NO: 137), operably linked to a termination sequence from the soybean oleosin gene. A separate expression cassette for the visual marker DsRed2B is used to identify transgenic seeds.

FIG. 5 shows a map for pED03 (SEQ ID NO: 154), a transformation vector designed for Agrobacterium-mediated transformation of dicots to express EDD and EDA genes from the algae Guillardia theta and Phaeodactylum tricornutum, respectively. The vector contains the soybean oleosin promoter (SEQ ID NO: 11), operably linked to a gene encoding EDD from Guillardia theta (SEQ ID NO: 140), operably linked to a termination sequence from the soybean oleosin gene. A second expression cassette contains the soybean oleosin promoter, operably linked to a gene encoding EDA from Phaeodactylum tricornutum (SEQ ID NO: 92), operably linked to a termination sequence from the soybean oleosin gene. A separate expression cassette for the visual marker DsRed2B is used to identify transgenic seeds.

FIG. 6 shows a map for pED04 (SEQ ID NO: 155), a transformation vector designed for Agrobacterium-mediated transformation of dicots to express EDD and EDA genes from Hordeum vulgare (barley). The vector contains the soybean oleosin promoter (SEQ ID NO: 11), operably linked to a gene encoding EDD from Hordeum vulgare (SEQ ID NO: 139), operably linked to a termination sequence from the soybean oleosin gene. A second expression cassette contains the soybean oleosin promoter, operably linked to a gene encoding EDA from Hordeum vulgare (SEQ ID NO: 97), operably linked to a termination sequence from the soybean oleosin gene. A separate expression cassette for the visual marker DsRed2B is used to identify transgenic seeds.

FIG. 7 shows a map for pED05 (SEQ ID NO: 156), a transformation vector designed for Agrobacterium-mediated transformation of dicots to express EDD and EDA genes from Zymomonas mobilis. The vector contains the SUS2 promoter from Arabidopsis thaliana (SEQ ID NO: 151) (van Erp et al. 2014; Plant Physiol. Vol. 165, pp 30-36), operably linked to the signal peptide coding sequence of the small subunit of Rubisco from pea (Cashmore, 1983), operably linked to a gene encoding EDD from Zymomonas mobilis protein (SEQ ID NO: 44), operably linked to a termination sequence from the soybean oleosin gene. A second expression cassette contains the SUS2 promoter from Arabidopsis, operably linked to the signal peptide coding sequence of the small subunit of Rubisco from pea, operably linked to a gene encoding EDA from Zymomonas mobilis protein (SEQ ID NO: 70), operably linked to a termination sequence from the soybean oleosin gene. A separate expression cassette for the visual marker DsRed2B is used to identify transgenic seeds.

FIG. 8 shows a map for pED06 (SEQ ID NO: 157), a transformation vector designed for Agrobacterium-mediated transformation of dicots to express EDD and EDA genes from the cyanobacteria Synechocystis sp. PCC 6803. The vector contains the SUS2 promoter from Arabidopsis (SEQ ID NO: 151), operably linked to the signal peptide of the small subunit of Rubisco from pea (Cashmore, 1983), operably linked to a gene encoding EDD from Synechocystis sp. PCC 6803 (SEQ ID NO: 136), operably linked to a termination sequence from the soybean oleosin gene. A second expression cassette contains the soybean oleosin promoter, operably linked to the signal peptide of the small subunit of Rubisco from pea, operably linked to a gene encoding EDA from Synechocystis sp. PCC 6803 protein (SEQ ID NO: 137), operably linked to a termination sequence from the soybean oleosin gene. A separate expression cassette for the visual marker DsRed2B is used to identify transgenic seeds.

FIG. 9 shows a map for pED07 (SEQ ID NO: 158), a transformation vector designed for Agrobacterium-mediated transformation of dicots to express EDD and EDA genes from the algae Guillardia theta and Phaeodactylum tricornutum, respectively. The vector contains the SUS2 promoter from Arabidopsis (SEQ ID NO: 151), operably linked to a gene encoding EDD from Guillardia theta (SEQ ID NO: 140), operably linked to a termination sequence from the soybean oleosin gene. A second expression cassette contains the SUS2 promoter from Arabidopsis, operably linked to a gene encoding EDA from Phaeodactylum tricornutum (SEQ ID NO: 92), operably linked to a termination sequence from the soybean oleosin gene. A separate expression cassette for the visual marker DsRed2B is used to identify transgenic seeds.

FIG. 10 shows a map for pED08 (SEQ ID NO: 159), a transformation vector designed for Agrobacterium-mediated transformation of dicots to express EDD and EDA genes from Hordeum vulgare (barley). The vector contains the SUS2 promoter from Arabidopsis (SEQ ID NO: 151), operably linked to a gene encoding EDD from Hordeum vulgare (SEQ ID NO: 139), operably linked to a termination sequence from the soybean oleosin gene. A second expression cassette contains the SUS2 promoter from Arabidopsis, operably linked to a gene encoding EDA from Hordeum vulgare (SEQ ID NO: 97), operably linked to a termination sequence from the soybean oleosin gene. A separate expression cassette for the visual marker DsRed2B is used to identify transgenic seeds.

### DETAILED DESCRIPTION OF THE INVENTION

Plant cells, tissues, and plants with modulated expression, preferably increased expression, of 6-phosphogluconate dehydratase (EDD; EC 4.2.1.12) and/or 2-keto-3-deoxy-6-phosphogluconate aldolase (EDA; EC 4.1.2.14) genes are disclosed. In preferred embodiments, the plant cells, tissues, and plants exhibit increased expression of EDD and EDA genes such that the rate of conversion of 6-phosphogluconate to pyruvate in the plastid is increased, resulting in increased crop performance and/or yield. The genes encoding the EDD and EDA enzymes can be used alone or in combination with altered expression of additional genes to enhance photosynthesis or carbon partitioning to seed. The expression of the genes encoding the EDD and EDA proteins can be increased using genetic engineering techniques to develop plants with increased performance and/or yield. Where genetic engineering techniques are used to increase the expression of the EDD and EDA proteins, the increased expression can be accomplished using transgenic technologies. The EDD and EDA genes can be expressed and the EDD and EDA proteins targeted to the plant plastid alone or in combinations with other genes that increase photosynthesis or carbon conversion efficiency within the seed.

In general, the key elements of crop yield and in particular seed, fruit or tuber yield can be divided into two parts: photosynthetic carbon capture to produce sucrose in the green tissue is referred to as the carbon source; followed by the transfer of carbon in the form of sucrose to the developing seed, fruit or tuber tissue which is referred to as the carbon sink. The flow of carbon from source tissue to sink tissue is subject to complex regulatory mechanisms. Increasing the seed fruit or tuber yield of a given crop is therefore dependent not only on improving photosynthetic efficiency in the source tissue but also increasing the strength of the sink tissue to pull fixed carbon into the development of seeds, fruit or tubers (also termed sink strength). Sink strength is in turn dependent on the metabolic processes taking place there. Pathways that lead to production of pyruvate, such as the Entner-Doudoroff pathway (also termed ED pathway), the Embden-Meyerhof-Parnas pathway (also termed glycolytic pathway), and the oxidative pentose phosphate pathway (also termed OPP pathway) provide metabolic building blocks for fatty acid biosynthesis as well as energy for seed, fruit or tuber biosynthesis.

In many crops, the factor that limits yield under nutrient-sufficient conditions is sink strength, or the rate at which phloem-supplied carbohydrates and amino acids are consumed by the seed. Often the overall metabolism of the seed produces more NAD(P)H and/or ATP than is actually required for the production of seed biomass. Some of the metabolic pathways responsible for producing these cofactors could be made more thermodynamically favorable, and thus likely faster, if they could ultimately accomplish the same conversion of substrate to product without reducing NAD(P)⁺ or phosphorylating ADP. This type of strategy can be employed up to a certain point in a seed without reducing its carbon conversion efficiency (CCE; moles of carbon in biomass per mole of carbon in phloem-supplied substrates), yet speeding up the overall metabolism to deplete phloem substrates more quickly and thus increasing demand for photosynthate. In some plants, utilizing this strategy can lead to increased carbon conversion efficiency. Many crops will respond to this increased demand for photosynthate by increasing their rate of photosynthesis, as there is generally substantially more capacity to produce photosynthate from CO₂ than the capacity to metabolize the photosynthate downstream in sink tissues such as the seed. The Entner-Doudoroff pathway is an example of a metabolic modification in the seed that could provide these advantages to seed yield.

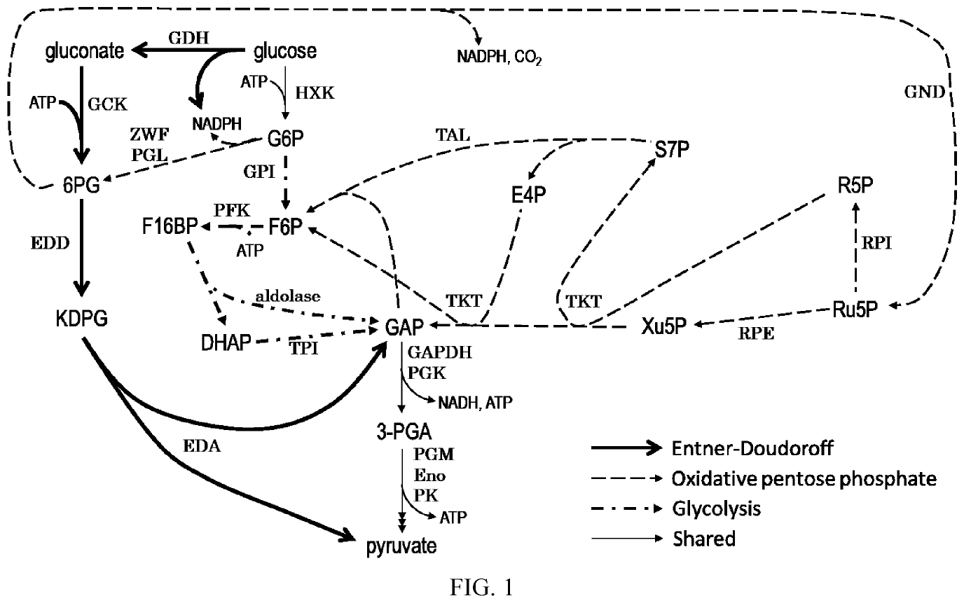

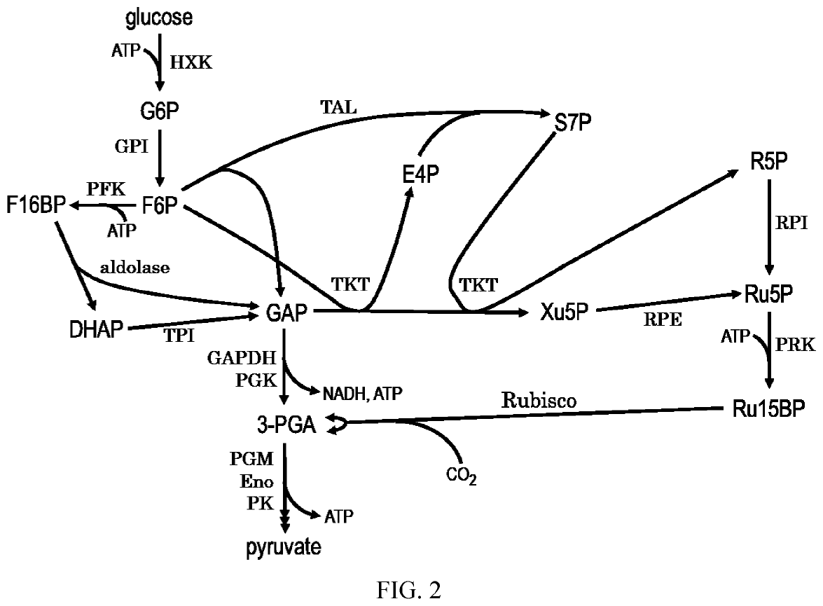

The Entner-Doudoroff pathway may be particularly suitable for increasing the carbon conversion efficiency of some seeds. For example, the Camelina sativa oilseed has a poor carbon conversion efficiency, and the reason for this has recently been determined to be a high flux through the oxidative pentose phosphate pathway (Carey et al., 2020, Plant Physiol. 182:493-506). Under conditions mimicking physiological light (10 μmol m⁻² s⁻¹ reaching the seed), C. sativa embryos in culture showed a carbon conversion efficiency of around 30%, compared to the closely related Brassica napus, whose embryos under similar conditions had a carbon conversion efficiency of around 80% (Goffman et al., 2005, Plant Physiol. 138:2269-2279). The OPP pathway is largely the reverse of the carbon-fixing Calvin cycle, and thus it produces rather than consumes CO₂. Metabolic flux labeling experiments have shown that canola uses Rubisco, without employing the Calvin cycle, to recapture CO₂ released in the seed increasing its carbon conversion efficiency (FIG. 2) (Schwender et al., Nature, 432, 779 (2004)). Similar metabolic flux labeling experiments in Camelina have shown that it does not recapture CO₂ in the seed with Rubisco, likely leading to its decreased carbon conversion efficiency compared to canola (Carey et al., 2020). When illuminated, embryonic cultures of C. sativa do not induce carbon fixation pathways associated with Rubisco; instead a subtle shift from the OPP pathway to the Embden-Meyerhof-Parnas (glycolytic) pathway (FIG. 1) begins to occur, and neither the reasons nor the mechanisms for this are clear (Carey et al., 2020). Furthermore, while Arabidopsis thaliana, a species closely related to C. sativa, has plentiful Rubisco expression in its siliques (Arabidopsis eFP Browser 2.0, website: bar.utoronto.ca/efp2/Arabidopsis/Arabidopsis_eFPBrowser2.html), C. sativa has very little, especially as the seed matures (Camelina eFP Browser, website: bar.utoronto.ca/efp_camelina/cgi-bin/efpWeb.cgi).

The Entner-Doudoroff pathway can be used to reroute carbon metabolism to increase carbon conversion efficiency. For example, in order to improve the carbon conversion efficiency of the camelina seed, the OPP flux can be displaced partially or entirely by one or more alternative pathways: the Embden-Meyerhof-Parnas (glycolytic) pathway, the Entner-Doudoroff (ED) pathway, and/or the use of Rubisco without a Calvin cycle. FIG. 1 shows the ED, OPP, and glycolytic pathways, while FIG. 2 shows the pathway utilizing Rubisco without the Calvin cycle.

The oxidation of phloem-supplied sucrose in plant seeds has been widely assumed to operate via the EMP pathway and by the OPP pathway. The ED pathway, however, was recently found to play at least some role in barley seedling metabolism (Chen et al., 2016, Proc. Natl. Acad. Sci. USA 113:5441-5446). In addition to improving the carbon conversion efficiency of a camelina oilseed, an engineered or upregulated ED pathway may also provide an increased rate of metabolism, or sink strength in camelina as well as other plants. The sink-strength benefit of the Entner-Doudoroff pathway is its limited production of NAD(P)H and ATP when compared to the other two pathways. For each glucose consumed, the EMP pathway has a net production of 2 NADH, 2 ATP and 2 pyruvate; the OPP pathway has a net production of 2 NADPH, 1.67 NADH, 1.67 ATP, and 1.67 pyruvate; and the ED pathway has a net production of 1 NADPH, 1 NADH, 1 ATP, and 2 pyruvate. Therefore the ED pathway has the lowest overall production of ATP and NAD(P)H and is therefore likely to be traversed by carbon more quickly than the others overall. In addition, the overall ED pathway has the fewest individual reactions per glucose consumed that produce either NAD(P)H or ATP, making it the least likely to encounter kinetic difficulties due to cofactor imbalances (FIGS. 1 and 2).

### EDD and EDA Proteins and Genes

Many plants have a plausible ortholog to one or more of the ED genes, but it is far from clear to what extent they actually affect plant metabolism. Homologous genes, also termed homologs, are two or more genes whose sequences are significantly related because of a close evolutionary relationship. Homologs occurring within a species are termed paralogs. Homologs occurring between species are termed orthologs. Sequence alignments can be used to identify homologs, based for example on the degree of identity or similarity of the sequences.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percent sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

In TABLE 1, the best plant match of the Entner-Doudoroff pathway enzymes to the E. coli enzymes are shown. Some plants do not appear to have a complete ED pathway, such as Camelina sativa, which lacks a good match to the gene encoding EDA (TABLE 1).

TABLE 1
Enzymes of the Entner-Doudoroff pathway.

| | EC | E. coli | Best Camelina | E | Best plant | | E |
|---|---|---|---|---|---|---|---|
| Enzyme | number | protein | match* | value | match* | Species | value |
| Glucose | 1.1.5.2 | NP_414666 | none | n/a | BHE74_00002036 | Ensete | 0.0 |
| dehydrogenase (GDH) | | (SEQ ID NO: 32) | | | (SEQ ID NO: 36) | ventricosum | |
| Gluconate kinase | 2.7.1.12 | NP_417894 | XP_019092454.1 | 1e−28 | XP_023902452.1 | Quercus suber | 2e−32 |
| (GCK) | | (SEQ ID NO: 33) | | | (SEQ ID NO: 37) | | |
| Phosphogluconate | 4.2.1.12 | NP_416365 | XP_010466820.1 | 2e−47 | KZV14789.1 | Dorcoceras | 0.0 |
| dehydratase (EDD) | | (SEQ ID NO: 34) | | | (SEQ ID NO: 38) | hygrometricum | |
| KDPG | 4.1.2.14 | NP_416364 | none | n/a | EEF26592.1 | Ricinus | 2e−62 |
| aldolase (EDA) | | (SEQ ID NO: 35) | | | (SEQ ID NO: 39) | communis | |

*Best BLAST match to the E. coli enzyme

The simplest way to engineer the ED pathway in seed tissue is to express the genes encoding EDD and EDA, each modified with a plastid targeting signal, under the control of a seed-specific promoter. This will direct the EDD and EDA proteins to seed plastids. It may not be necessary to express GDH and GCK, as 6-phosphogluconate is already produced by the plastidic ZWF protein of the OPP pathway, which is dominant in camelina seeds (Carey et al., 2020, Plant Physiol. 182:493-506). However, additional expression of GDH and/or GCK could encourage glucose to be catabolized by the ED pathway instead of the EMP pathway. Cytosolic expression of EDD and EDA could also be beneficial, as plants like A. thaliana have at least some cytosolic ZWF activity (Wakao et al., 2008, Plant Physiol. 146:277-288).

For advantageous expression of EDD and EDA in plants, one would seek genes whose protein products are subject to minimal regulation in the plant, and these are most likely to originate in bacteria or algae. However, there could also be advantages to sourcing the EDD and EDA genes from plants, so that government regulatory hurdles are minimized. The ED pathway is customarily associated with heterotrophic bacteria, but it was recently found that the pathway may operate significantly in cyanobacteria and even higher plants (Chen et al., 2016, Proc. Natl. Acad. Sci. USA 113:5441-5446).

One attempt to replace the EMP pathway in the yeast Saccharomyces cerevisiae with the ED pathway from Escherichia coli was not successful due to the low activity of the EDD gene product which appeared to be related to assembly of the required iron-sulfur cluster (Benisch et al., 2014, J. Biotechnol. 171:45-55). The low activity of the ED pathway using such an approach was also observed by Morita et al., 2017, J. Biosci. Bioeng. 124:263-270. Given these challenges it may be preferred to implement an ED pathway using genes of plant origin, or alternatively, use a pathway from another photosynthetic organism such as cyanobacteria.

TABLE 2 gives representative examples of bacterial EDD proteins. Genes encoding EDD are found in numerous bacteria, thus TABLE 2 only lists preferred sources of EDD, such as bacteria used in food preparation.

TABLE 2
6-Phosphogluconate dehydratase (EDD)
proteins from benign bacteria.

| Species | GenBank accession | SEQ ID NO. |
|---|---|---|
| Acetobacter aceti | AQS83953 | 40 |
| Hafnia alvei | AIU72652 | 41 |
| Proteus vulgaris | ATM99262 | 42 |
| Pseudomonas fluorescens | ABA76112 | 43 |
| Zymomonas mobilis | AAV88992 | 44 |

TABLE 3 gives representative examples of plant and algal EDD proteins. These are the top hits from a protein BLAST search using the Escherichia coli EDD protein (NCBI Reference Sequence: NP_416365.1) as the query sequence.

TABLE 3
6-Phosphogluconate dehydratase (EDD)
proteins from algae and plants.

| | GenBank | SEQ | |
|---|---|---|---|
| Species | accession | ID NO. | E value |
| Dorcoceras hygrometricum | KZV14789.1 | 38 | 0.0 |
| Chlamydomonas eustigma | GAX72671.1 | 45 | 4e−52 |
| Manihot esculenta | XP_021594917.1 | 46 | 2e−51 |
| Chlorella sorokiniana | PRW61595.1 | 47 | 2e−51 |
| Quercus suber | XP_023898230.1 | 48 | 1e−50 |
| Physcomitrella patens | XP_024389039.1 | 49 | 2e−50 |
| Quercus suber | XP_023903590.1 | 50 | 6e−50 |
| Nicotiana tomentosiformis | XP_009616638.2 | 51 | 8e−50 |
| Micractinium conductrix | PSC73296.1 | 52 | 8e−50 |
| Nicotiana attenuata | XP_019249905.1 | 53 | 9e−50 |

TABLE 4 gives representative examples of cyanobacterial EDD proteins. These are the top hits from a protein BLAST search using the Escherichia coli EDD protein (NCBI Reference Sequence: NP_416365.1) as the query sequence.

TABLE 4
Phosphogluconate dehydratase (EDD) proteins from cyanobacteria.

| | GenBank | SEQ | |
|---|---|---|---|
| Species | accession | ID NO. | E value |
| Nostoc sp. 3335mG | WP_110151523.1 | 54 | 0.0 |
| Nostoc sp. 3335mG | WP_110156247.1 | 55 | 0.0 |
| Nostoc sp. 3335mG | WP_110153205.1 | 56 | 0.0 |
| Cyanobacteria bacterium | HCB11380.1 | 57 | 1e−71 |
| UBA11991 | | | |
| Cyanobacteria bacterium | HBG49484.1 | 58 | 5e−70 |
| Cyanobacteria bacterium | HAS94029.1 | 59 | 6e−70 |
| Cyanobacteria bacterium | HBH18928.1 | 60 | 8e−67 |
| Planktothricoidea sp. SR001 | WP_054466347.1 | 61 | 8e−66 |
| Planktothrix paucivesiculata | CUR17874.1 | 62 | 2e−65 |
| PCC 9631 | | | |
| Oscillatoriales bacterium | HBW56505.1 | 63 | 3e−65 |
| UBA8482 | | | |

TABLE 5 gives representative examples of bacterial EDA proteins. Genes encoding EDA are found in numerous bacteria, thus TABLE 5 only lists preferred sources of EDA, such as bacteria used in food preparation.

TABLE 5
KDPG aldolase (EDA) proteins from benign bacteria.

| | GenBank | SEQ |
|---|---|---|
| Species | accession | ID NO. |
| Acetobacter aceti | AQS83954 | 64 |
| Hafnia alvei | AIU72282, AIU72653 | 65, 66 |
| Proteus vulgaris | ATM99261, ATM99743 | 67, 68 |
| Pseudomonas fluorescens | ABA76098 | 69 |
| Zymomonas mobilis | AAV89621 | 70 |

TABLE 6 gives representative examples of cyanobacterial EDA proteins. These are the top hits from a protein BLAST search using the Escherichia coli EDA protein (NCBI Reference Sequence: NP_416364.1) as the query sequence.

TABLE 6
KDPG aldolase (EDA) proteins from cyanobacteria.

| | GenBank | SEQ | |
|---|---|---|---|
| Species | accession | ID NO. | E value |
| Leptolyngbya valderiana | WP_063717938.1 | 71 | 9e−68 |
| Nostoc sp. 3335mG | WP_110154617.1 | 72 | 6e−61 |
| Nostoc sp. 3335mG | WP_110149078.1 | 73 | 2e−60 |
| Nostoc sp. 3335mG | WP_110153203.1 | 74 | 3e−55 |
| cyanobacterium endosymbiont | WP_119261026.1 | 75 | 1e−33 |
| of Rhopalodia gibberula | | | |
| cyanobacterium endosymbiont | BBA80122.1 | 76 | 2e−33 |
| of Rhopalodia gibberula | | | |
| Coleofasciculus chthonoplastes | EDX74995.1 | 77 | 2e−33 |
| PCC 7420 | | | |
| Coleofasciculus chthonoplastes | WP_044207780.1 | 78 | 2e−33 |
| unclassified Calothrix | WP_096691521.1 | 79 | 2e−32 |
| Crocosphaera watsonii | WP_007305875.1 | 80 | 4e−32 |

TABLE 7 is adapted from Chen et al., 2016, Proc. Natl. Acad. Sci. USA 113:5441-5446, and it outlines candidates for EDA proteins found in algae and plants.

TABLE 7
Candidate KDPG aldolase (EDA)-encoding
genes from algae and plants.

| | GenBank | SEQ |
|---|---|---|
| Species | accession | ID NO. |
| Aureococcus anophagefferens | XP_009040683 | 81 |
| Bathycoccus prasinos | XP_007512337 | 82 |
| Cyanidioschyzon merolae strain 10D | XP_005537929 | 83 |
| Ectocarpus siliculosus | CBN76672 | 84 |
| Emitiania huxleyi CCMP1516 | XP_005764518 | 85 |
| Galdieria sulphuraria | XP_005704546 | 86 |
| Guillardia theta | XP_005827436 | 87 |
| Micromonas pusilia CCMP1545 | XP_003055404 | 88 |
| Micromonas sp. RCC299 | XP_002507190 | 89 |
| Ostreococcus lucimarinus CCE9901 | XP_001420689 | 90 |
| Ostreococcus tauri | XP_003082415 | 91 |
| Phaeodactylum tricornutum CCAP 1055/1 | XP_002178649 | 92 |
| Thalassiosira oceanica | EJK68780 | 93 |
| Thalassiosira pseudonana CCMP1335 | XP_002295264 | 94 |
| Physcomitrella patens | XP_001755760 | 95 |
| Selaginella moellendorffii | XP_002976272 | 96 |
| Hordeum vulgare | BAJ87430 | 97 |
| Amborella trichopoda | XP_011620750 | 98 |
| | ERM99071 | 99 |
| Beta vulgaris subsp. vulgaris | XP_010670094 | 100 |
| Brachypodium distachyon | XP_003557377 | 101 |
| Cicer arietinum | XP_004489172 | 102 |
| Cucumis melo | XP_008445938 | 103 |
| Cucumis sativus | XP_011655529 | 104 |
| Erythranthe guttatus | XP_012839517 | 105 |
| Eucalyptus grandis | XP_010068944 | 106 |
| Fragaria vesca subsp. vesca | XP_004308199 | 107 |
| Genlisea aurea | EPS68767 | 108 |
| Glycine max | XP_006587762 | 109 |
| | XP_003534460 | 110 |
| Gossypium raimondii | XP_012455519 | 111 |
| Jatropha curcas | XP_012068735 | 112 |
| Medicago truncatula | XP_003621137 | 113 |
| Musa acuminata subsp. malaccensis | XP_009410424 | 114 |
| Nelumbo nucifera | XP_010251921 | 115 |
| | XP_010251919 | 116 |
| Nicotiana sylvestris | XP_009777068 | 117 |
| Nicotiana tomentosiformis | XP_009619331 | 118 |
| Oryza brachyantha | XP_006657563 | 119 |
| Oryza sativa Indica Group | EEC81744 | 120 |
| Oryza sativa Japonica Group | BAC45190 | 121 |
| Phaseolus vulgaris | XP_007139597 | 122 |
| Phoenix dactylifera | XP_008788570 | 123 |
| Populus euphratica | XP_011034685 | 124 |
| Populus trichocarpa | XP_002299177 | 125 |
| Ricinus communis | XP_002525556 | 126 |
| Sesamum indicum | XP_011069998 | 127 |
| Setaria italica | XP_004955816 | 128 |
| Solanum lycopersicum | XP_004249354 | 129 |
| Solanum tuberosum | XP_006339220 | 130 |
| Sorghum bicolor | XP_002459551 | 131 |
| Spinacia oleracea | KNA15727 | 132 |
| Theobroma cacao | XP_007010757 | 133 |
| Triticum urartu | EMS48606 | 134 |
| Zea mays | NP_001150487 | 135 |

It should further be noted that Chen et al., 2016, Proc. Natl. Acad. Sci. USA 113:5441-5446 identified cyanobacterial proteins that catalyze EDD and EDA reactions by direct assay. These are the EDD (GenBank accession BAA18807; SEQ ID NO: 136) and EDA (GenBank accession BAA10632; SEQ ID NO: 137) proteins of Synechocystis sp. PCC 6803, encoded by the slr0452 and sll0107 genes, respectively. In addition, Fabris et al., 2012, Plant J. 70:1004-1014 identified a functional EDD-EDA pair in the diatom Phaeodactylum tricornutum by complementation of an Escherichia coli mutant lacking the ED pathway. The EDD (XP_002180649, SEQ ID NO: 138) and EDA (XP_002178649, SEQ ID NO: 92) proteins in this organism are encoded by the PHATRDRAFT 20547 and PHATRDRAFT 34120 loci, respectively. While Chen et al., 2016, Proc. Natl. Acad. Sci. USA 113:5441-5446 did identify and assay an EDA from Hordeum vulgare (barley), shown in TABLE 7, they did not identify its EDD counterpart. A BLAST search using the Escherichia coli protein b1851 (EDD) reveals that the most likely H. vulgare candidate to possess EDD activity is the protein KAE8808450 (SEQ ID NO: 139), with an E value of 5e-46.

EDD and EDA genes from any source can be used, but in most cases it is preferable for the plant to be genetically engineered to increase expression of the EDD and EDA proteins in the plastid of the plant cells.

Accordingly, disclosed herein is a genetically engineered plant that expresses a 6-phosphogluconate dehydratase (EDD) and/or a 2-keto-3-deoxy-6-phosphogluconate aldolase (EDA). Preferably the genetically engineered plant has modulated and/or increased expression of one or more EDD and EDA proteins. Preferably the genetically engineered plant has increased expression of one or more EDD and EDA proteins in the plastid and has higher performance, seed, fruit or tuber yield, and/or seed oil content. In a preferred embodiment the expression of the EDD and EDA protein is directed from a plant seed specific or seed—preferred promoter.

The genetically engineered plant comprises at least one of a first modified gene or a second modified gene.

The first modified gene comprises (i) a first promoter and (ii) a nucleic acid sequence encoding the EDD.

The first promoter is non-cognate with respect to the nucleic acid sequence encoding the EDD. A promoter that is non-cognate with respect to a nucleic acid sequence means that the promoter is not naturally paired with the nucleic acid sequence in organisms from which the promoter and/or the nucleic acid sequence are derived. Instead, the promoter has been paired with the nucleic acid sequence based on use of recombinant DNA techniques to create a modified gene.

The first modified gene is configured such that transcription of the nucleic acid sequence encoding the EDD is initiated from the first promoter and results in expression of the EDD. Accordingly, in the context of the first modified gene, the promoter functions as a promoter of transcription of the nucleic acid sequence, and thus of expression of the EDD. In preferred examples, the expression of the EDD is higher in the genetically engineered plant than in a corresponding reference plant that does not include the first modified gene.

Similarly as for the first modified gene, the second modified gene comprises (i) a second promoter and (ii) a nucleic acid sequence encoding the EDA. The second promoter is non-cognate with respect to the nucleic acid sequence encoding the EDA, i.e. the promoter is not naturally paired with the nucleic acid sequence in organisms from which the promoter and/or the nucleic acid sequence are derived. The second modified gene is configured such that transcription of the nucleic acid sequence encoding the EDA is initiated from the second promoter and results in expression of the EDA. Accordingly, in the context of the second modified gene, the promoter functions as a promoter of transcription of the nucleic acid sequence, and thus of expression of the EDA. In preferred examples, the expression of the EDA is higher in the genetically engineered plant than in a corresponding reference plant that does not include the second modified gene.

In some embodiments, the EDD is characterized as EC 4.2.1.12. In some embodiments, the EDD converts 6-phosphogluconate (6PG) to 2-keto-3-deoxy-6-phosphogluconate (KDPG) and water. In some embodiments, the EDD is one or more of a bacterial EDD, a cyanobacterial EDD, an algal EDD, or a plant EDD. In some embodiments, the EDD has at least 30% or higher, at least 40% or higher, at least 50% or higher, at least 60% or higher, at least 70% or higher, at least 80% or higher, at least 90% or higher, at least 95% or higher, at least 96% or higher, at least 97% or higher, at least 98% or higher, or at least 99% or higher sequence identity to one or more of the following: (1) Zymomonas mobilis EDD of SEQ ID NO: 44; (2) Synechocystis sp. PCC 6803 EDD of SEQ ID NO: 136; (3) Guillardia theta EDD of SEQ ID NO: 140; or (4) Hordeum vulgare EDD of SEQ ID NO: 139. In some embodiments, the EDD comprises one or more of the following: (1) Zymomonas mobilis EDD of SEQ ID NO: 44; (2) Synechocystis sp. PCC 6803 EDD of SEQ ID NO: 136; (3) Guillardia theta EDD of SEQ ID NO: 140; or (4) Hordeum vulgare EDD of SEQ ID NO: 139.

In some embodiments, the EDA is characterized as EC 4.1.2.14. In some embodiments, the EDA converts 2-keto-3-deoxy-6-phosphogluconate (KDPG) to pyruvate and D-glyceraldehyde 3-phosphate. In some embodiments, the EDA is one or more of a bacterial EDA, a cyanobacterial EDA, an algal EDA, or a plant EDA. In some embodiments, the EDA has at least 30% or higher, at least 40% or higher, at least 50% or higher, at least 60% or higher, at least 70% or higher, at least 80% or higher, at least 90% or higher, at least 95% or higher, at least 96% or higher, at least 97% or higher, at least 98% or higher, or at least 99% or higher sequence identity to one or more of the following: (1) Zymomonas mobilis EDA of SEQ ID NO: 70; (2) Synechocystis sp. PCC 6803 EDA of SEQ ID NO: 137; (3) Phaeodactylum tricornutum EDA of SEQ ID NO: 92; or (4) Hordeum vulgare EDA of SEQ ID NO: 97. In some embodiments, the EDA comprises one or more of the following: (1) Zymomonas mobilis EDA of SEQ ID NO: 70; (2) Synechocystis sp. PCC 6803 EDA of SEQ ID NO: 137; (3) Phaeodactylum tricornutum EDA of SEQ ID NO: 92; or (4) Hordeum vulgare EDA of SEQ ID NO: 97.

In some embodiments, the genetically engineered plant expresses both the EDD and the EDA. In some of the embodiments, the genetically engineered plant comprises both the first modified gene and the second modified gene. In others of these embodiments, the genetically engineered plant comprises the first modified gene, lacks the second modified gene, and further comprises an endogenous gene encoding the EDA. In still others of these embodiments, the genetically engineered plant lacks the first modified gene, comprises the second modified gene, and further comprises an endogenous gene encoding the EDD.

In some embodiments, the first promoter comprises one or more of a constitutive promoter, a seed-specific promoter, or a seed-preferred promoter. Suitable promoters are discussed below.

In some embodiments, the second promoter comprises one or more of a constitutive promoter, a seed-specific promoter, or a seed-preferred promoter.

In some embodiments, the genetically engineered plant exhibits modulated expression of the EDD and the EDA relative to a reference plant that does not comprise the at least one of the first modified gene or the second modified gene.

In some embodiments, the genetically engineered plant exhibits increased expression of the EDD and the EDA relative to a reference plant that does not comprise the at least one of the first modified gene or the second modified gene.

In some embodiments, the genetically engineered plant exhibits increased expression of the EDD and the EDA in plastids of cells of the genetically engineered plant relative to a reference plant that does not comprise the at least one of the first modified gene or the second modified gene.

In some embodiments, the first modified gene further comprises a nucleic acid sequence encoding a plastid targeting sequence and is further configured such that the EDD comprises an N-terminal plastid targeting signal, and the second modified gene further comprises a nucleic acid sequence encoding a plastid targeting sequence and is further configured such that the EDA comprises an N-terminal plastid targeting signal.

As noted above, the simplest way to engineer the ED pathway in seed tissue is to express the genes encoding EDD and EDA, each modified with a plastid targeting signal, under the control of a seed-specific promoter. As also noted, it may not be necessary to express GDH and GCK, as 6-phosphogluconate is already produced by the plastidic ZWF protein of the OPP pathway, which is dominant in camelina seeds (Carey et al., 2020, Plant Physiol. 182:493-506). Yet, additional expression of GDH and/or GCK could encourage glucose to be catabolized by the ED pathway instead of the EMP pathway. Moreover, cytosolic expression of EDD and EDA could also be beneficial, as plants like A. thaliana have at least some cytosolic ZWF activity (Wakao et al., 2008, Plant Physiol. 146:277-288).

Thus, in some embodiments, the genetically engineered plant further comprises one or more additional modified genes, each of the one or more additional modified genes comprising (i) a respective promoter and (ii) a respective nucleic acid sequence encoding one or more of glucose-6-phosphate dehydrogenase (ZWF), 6-phosphogluconolactonase (PGL), glucose dehydrogenase (GDH), or gluconate kinase (GCK), each respective promoter being non-cognate with respect to its respective nucleic acid sequence encoding the one or more of ZWF, PGL, GDH, or GCK, and each additional modified gene being configured such that transcription of its respective nucleic acid sequence encoding the one or more of ZWF, PGL, GDH, or GCK is initiated from its respective promoter and results in expression of the one or more of ZWF, PGL, GDH, or GCK.

### Plants

A “plant,” as the term is used herein, generally refers to a plant belonging to the plant subkingdom Embryophyta, including higher plants, also termed vascular plants, and mosses, liverworts, and hornworts.

The term “plant” includes mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

Plants encompass all annual and perennial monocotyledonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Populus, Camelina, Beta, Solanum, and Carthamus. Preferred plants are those from the following plant families: Amaranthaceae, Asteraceae, Brassicaceae, Carophyllaceae, Chenopodiaceae, Compositae, Cruciferae, Cucurbitaceae, Euphorbiaceae, Fabaceae, Labiatae, Leguminosae, Papilionoideae, Liliaceae, Linaceae, Malvaceae, Poaceae, Rosaceae, Rubiaceae, Saxifragaceae, Scrophulariaceae, Solanaceae, Sterculiaceae, Tetragoniaceae, Theaceae, Umbelliferae.

The plant can be a monocotyledonous plant or a dicotyledonous plant. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit. Preferred monocotyledonous plants include maize, rice, wheat, sugarcane, sorghum, oats and barley.

Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize), and various nut species such as, for example, walnut or almond.

Camelina species, commonly known as false flax, are native to Mediterranean regions of Europe and Asia and seem to be particularly adapted to cold semiarid climate zones (steppes and prairies). The species Camelina sativa was historically cultivated as an oilseed crop to produce vegetable oil and animal feed. In addition to being useful as an industrial oilseed crop, Camelina is a very useful model system for developing new tools and genetically engineered approaches to enhancing the yield of crops in general and for enhancing the yield of seed and seed oil in particular. Demonstrated transgene improvements in Camelina can then be deployed in major oilseed crops including Brassica species including B. napus (canola), B. rapa, B. juncea, B. carinata, crambe, soybean, sunflower, safflower, oil palm, flax, and cotton.

As will be apparent, the plant can be a C3 photosynthesis plant, i.e. a plant in which Rubisco catalyzes carboxylation of ribulose-1,5-bisphosphate by use of CO₂ drawn directly from the atmosphere, such as for example, wheat, oat, and barley, among others. The plant also can be a C4 plant, i.e. a plant in which Rubisco catalyzes carboxylation of ribulose-1,5-bisphosphate by use of CO₂ shuttled via malate or aspartate from mesophyll cells to bundle sheath cells, such as for example maize, millet, and sorghum, among others.

Accordingly, in some embodiments the genetically engineered plant is a C3 plant. Also, in some embodiments the genetically engineered plant is a C4 plant. Also, in some embodiments the genetically engineered plant is an oilseed crop plant selected from the group consisting of Camelina sativa, camelina species, Brassica species (e.g. B. napus (canola), B. rapa, B. juncea, and B. carinata), crambe, soybean, sunflower, safflower, oil palm, flax, and cotton. Also, in some embodiments the genetically engineered plant is a major food or feed crop plant and/or a plant used in phytoremediation selected from the group consisting of maize, wheat, oat, barley, soybean, Brassica species, Brassica napus (canola), rapeseed, Brassica rapa, Brassica carinata, Brassica juncea, Thlaspi caerulescens (pennycress), sunflower, safflower, oil palm, millet, sorghum, potato, lentil, chickpea, pea, pulse, bean, tomato, and rice. In some of these embodiments, the genetically engineered plant is maize.

Thus, in some embodiments, the genetically engineered plant comprises one or more of Camelina sativa, camelina species, Brassica species, Brassica napus (canola), Brassica rapa, Brassica juncea, Brassica carinata, crambe, soybean, sunflower, safflower, oil palm, flax, or cotton. In some embodiments, the genetically engineered plant comprises one or more of maize, wheat, oat, barley, soybean, Brassica species, Brassica napus (canola), rapeseed, Brassica rapa, Brassica carinata, Brassica juncea, Thlaspi caerulescens (pennycress), sunflower, safflower, oil palm, millet, sorghum, potato, lentil, chickpea, pea, pulse, bean, tomato, or rice.

Modulated and/or Increased Expression of EDD and EDA Proteins

As noted above, in some embodiments, the genetically engineered plant exhibits modulated expression of the EDD and the EDA relative to a reference plant that does not comprise the at least one of the first modified gene or the second modified gene. In some embodiments, the genetically engineered plant exhibits increased expression of the EDD and the EDA relative to a reference plant that does not comprise the at least one of the first modified gene or the second modified gene.

In certain embodiments, a genetically engineered plant having increased expression of EDD and EDA can have a carbon conversion efficiency (CCE) that is higher than for a corresponding reference plant not having the increased expression of EDD and/or EDA. For example, the genetically engineered plant can have a carbon conversion efficiency that is at least 5% higher, at least 10% higher, at least 20% higher, at least 40% higher, at least 80% higher, at least 120% higher, or at least 160% higher than for a corresponding reference plant that does not have the increased expression of EDD and EDA.

A genetically engineered plant having increased expression of EDD and EDA also can have a seed, fruit or tuber yield that is higher than for a corresponding reference plant not having the increased expression of EDD and EDA. For example, the genetically engineered plant can have a seed yield that is at least 5% higher, at least 10% higher, at least 20% higher, at least 40% higher, at least 60% higher, or at least 80% higher, than for a corresponding reference plant that does not have the increased expression of EDD and EDA.

A genetically engineered plant having increased expression of EDD and EDA also can produce larger seeds, fruits or tubers than a corresponding reference plant not having the increased expression of EDD and EDA. For example, the genetically engineered plant can produce seeds, fruits or tubers that are at least 5% larger, at least 10% larger, at least 20% larger, at least 40% larger, at least 60% larger, or at least 80% larger, than for a corresponding reference plant that does not have the increased expression of EDD and EDA.

A genetically engineered plant having increased expression of EDD and EDA also can produce seeds with higher oil content than a corresponding reference plant not having the increased expression of EDD and EDA. For example, the genetically engineered plant can produce seeds with at least 5% more oil, at least 10% more oil, at least 20% more oil, at least 40% more oil, at least 60% more oil, or at least 80% more oil, than for a corresponding reference plant that does not have the increased expression of EDD and EDA.

A genetically engineered plant having increased expression of EDD and EDA can also produce an increased number of seeds, fruits or tubers than a corresponding reference plant not having the increased expression of EDD and EDA. For example, the genetically engineered plant can produce a number of seeds, fruits or tubers that is at least 5% higher, at least 10% higher, at least 20% higher, at least 40% higher, at least 60% higher, or at least 80% higher, than for a corresponding reference plant that does not have the increased expression of EDD and EDA.

Thus, in some embodiments, the genetically engineered plant has a carbon conversion efficiency that is at least 5% higher, at least 10% higher, at least 20% higher, at least 40% higher, at least 80% higher, at least 120% higher, or at least 160% higher than for a reference plant that does not comprise the at least one of the first modified gene or the second modified gene. In some embodiments, the genetically engineered plant has an increased sink strength in comparison to a reference plant that does not comprise the at least one of the first modified gene or the second modified gene.

In some embodiments, the genetically engineered plant has one or more characteristics selected from higher performance and/or seed, fruit or tuber yield relative to a reference plant that does not comprise the at least one of the first modified gene or the second modified gene. In some of these embodiments, the one or more characteristics are increased by 10%, 20%, 30%, 40%, 50%, 60%, 70%, 80%, 90%, 100% or higher relative to a reference plant that does not comprise the at least one of the first modified gene or the second modified gene.

### Methods of Making the Genetically Engineered Plant

As noted above, a method for producing the genetically engineered plant also is provided. The method comprises a step of: (1) introducing at least one of the first modified gene or the second modified gene into a plant, thereby obtaining the genetically engineered plant.

Following identification of suitable EDD and EDA proteins, a genetically engineered plant having increased expression of the EDD and EDA proteins in the plastid can be made by methods that are known in the art, for example as follows.

DNA constructs useful in the methods described herein include transformation vectors capable of introducing transgenes or other modified nucleic acid sequences into plants. As used herein, “genetically engineered” refers to an organism in which a nucleic acid fragment containing a heterologous nucleotide sequence has been introduced, or in which the expression of a homologous gene has been modified, for example by genome editing. Transgenes in the genetically engineered organism are preferably stable and inheritable. Heterologous nucleic acid fragments may or may not be integrated into the host genome.

Several plant transformation vector options are available, including those described in Gene Transfer to Plants, 1995, Potrykus et al., eds., Springer-Verlag Berlin Heidelberg New York, Genetically engineered Plants: A Production System for Industrial and Pharmaceutical Proteins, 1996, Owen et al., eds., John Wiley & Sons Ltd. England, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof. See, for example, U.S. Pat. No. 5,639,949.

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. See, for example, U.S. Pat. No. 5,639,949. Alternatively, DNA fragments containing the transgene and the necessary regulatory elements for expression of the transgene can be excised from a plasmid and delivered to the plant cell using microprojectile bombardment-mediated methods.

Zinc-finger nucleases (ZFNs) are also useful in that they allow double strand DNA cleavage at specific sites in plant chromosomes such that targeted gene insertion or deletion can be performed (Shukla et al., 2009, Nature 459: 437-441; Townsend et al., 2009, Nature 459: 442-445).

The CRISPR/Cas9 system (Sander, J. D. and Joung, J. K., Nature Biotechnology, published online Mar. 2, 2014; doi; 10.1038/nbt.2842) is particularly useful for editing plant genomes to modulate the expression of homologous genes encoding enzymes. All that is required to achieve a CRISPR/Cas edit is a Cas enzyme, or other CRISPR nuclease (Murugan et al. (2017), Mol Cell, 68:15), and a single guide RNA (sgRNA) as reviewed extensively by others (Belhag et al. (2015), Curr. Opin. Biotech., 32: 76; Khandagale & Nadaf (2016), Plant Biotechnol Rep, 10:327-343). Several examples of the use of this technology to edit the genomes of plants have now been reported (Belhaj et al. (2013), Plant Methods, 9:39; Zhang et al. (2016), Journal of Genetics and Genomics, 43: 251).

TALENs (transcriptional activator-like effector nucleases), meganucleases, or zinc finger nucleases (ZFNs) can also be used for plant genome editing (Malzahn et al., Cell Biosci, 2017, 7:21; Khandagal & Nadal, Plant Biotechnol Rep, 2016, 10, 327).

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998) (soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun for Agrisoma technology are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128). Methods for plant regeneration from protoplasts have also been described (Evans et al., in Handbook of Plant Cell Culture, Vol 1, (Macmillan Publishing Co., New York, 1983); Vasil, IK in Cell Culture and Somatic Cell Genetics (Academic, Orlando, 1984)).

Recombinase technologies which are useful for producing the disclosed genetically engineered plants include the cre-lox, FLP/FRT and Gin systems. Methods by which these technologies can be used for the purpose described herein are described for example in (U.S. Pat. No. 5,527,695; Dale and Ow, 1991, Proc. Natl. Acad. Sci. USA 88: 10558-10562; Medberry et al., 1995, Nucleic Acids Res. 23: 485-490).

Transformation protocols as well as protocols for introducing nucleotide sequences into plants may vary depending on the type of plant or plant cell, e.g., monocot or dicot, targeted for transformation.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain genetically engineered plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant 16: 735-743), transformation of germinating seeds (Feldmann & Marks, 1987, Mol. Gen. Genet. 208: 1-9), floral dip (Clough and Bent, 1998, Plant 16: 735-743), and floral spray (Chung et al., 2000, Genetically engineered Res. 9: 471-476). Other plants that have successfully been transformed by in planta methods include rapeseed and radish (vacuum infiltration, Ian and Hong, 2001, Genetically engineered Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Genetically engineered plants can be produced using conventional techniques to express any genes of interest in plants or plant cells (Methods in Molecular Biology, 2005, vol. 286, Genetically engineered Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

In some embodiments, the heterologous polynucleotides of the invention can be transformed into the nucleus using standard techniques known in the art of plant transformation.

Thus, in some embodiments, a heterologous polynucleotide encoding a phosphogluconate dehydratase or 2-keto-3-deoxy-6-phosphogluconate aldolase polypeptide can be transformed into and expressed in the nucleus and the polypeptides produced remain in the cytosol. In other embodiments, a heterologous polynucleotide encoding a phosphogluconate dehydratase or 2-keto-3-deoxy-6-phosphogluconate aldolase polynucleotide can be transformed into and expressed in the nucleus, wherein the polypeptides can be targeted to the plastid. Thus, in particular embodiments, a heterologous polynucleotide encoding a phosphogluconate dehydratase or 2-keto-3-deoxy-6-phosphogluconate aldolase polypeptide can be operably linked to at least one targeting nucleotide sequence encoding a signal peptide that targets the polypeptides to the plastid.

In some embodiments, the step (1) comprises transforming the plant cell of the plant with the at least one of the first modified gene or the second modified gene. In some embodiments, the step (1) comprises transforming the plant cell of the plant with both the first modified gene and the second modified gene.

In some embodiments, the method further comprising steps of: (2) selecting the transformed plant cell on a selective medium; (3) regenerating the selected transformed plant cell to produce a differentiated plant; and (4) selecting the differentiated plant based on expression of the at least one of the first modified gene or the second modified gene in at least one of a tissue or a cellular location of the differentiated plant, thereby obtaining the genetically engineered plant.

### Plastid Targeting Sequences

Plastid targeting sequences are well known in the art and include, for example, the chloroplast small subunit of ribulose-1,5-bisphosphate carboxylase (Rubisco) (de Castro Silva Filho et al. Plant Mol. Biol. 30:769-780 (1996); Schnell et al. J. Biol. Chem. 266(5):3335-3342 (1991)); 5-(enolpyruvyl)shikimate-3-phosphate synthase (EPSPS) (Archer et al. J Bioenerg. Biomemb. 22(6):789-810 (1990)); tryptophan synthase (Zhao et al. J. Biol. Chem. 270(11):6081-6087 (1995)); plastocyanin (Lawrence et al. J. Biol. Chem. 272(33):20357-20363 (1997)); chorismate synthase (Schmidt et al. J. Biol. Chem. 268(36):27447-27457 (1993)); and the light harvesting chlorophyll a/b binding protein (LHBP) (Lamppa et al. J. Biol. Chem. 263:14996-14999 (1988)). See also Von Heijne et al. Plant Mot Biol. Rep. 9:104-126 (1991); Clark et al. J Biol. Chem. 264:17544-17550 (1989); Della-Cioppa et al. Plant Physiol. 84:965-968 (1987); Romer et al. Biochem. Biophys. Res. Commun. 196:1414-1421 (1993); and Shah et al. Science 233:478-481 (1986). Alternative plastid targeting signals have also been described in the following: US 2008/0263728; Miras, S. et al. (2002), J Biol Chem 277(49): 47770-8; Miras, S. et al. (2007), J Biol Chem 282: 29482-29492.

Specific examples of using N-terminal plastid targeting sequences to target microbial proteins to plant plastids are disclosed for example by Malik et al., Plant Biotechnol. J., 13:675 (2015) and Petrasovits et al., Plant Biotechnol. J., 5:162 (2007).

Signal peptides (and the targeting nucleotide sequences encoding them) can be found in public databases such as the “Signal Peptide Website: An Information Platform for Signal Sequences and Signal Peptides.” (website: signalpeptide.de); the “Signal Peptide Database” (website: proline.bic.nus.edu.sg/spdb/index.html) (Choo et al., BMC Bioinformatics 6:249 (2005) (available on website: biomedcentral.com/1471-2105/6/249/abstract); Predotar (urgi.versailles.inra.fr/predotar/predotar.html; predicts mitochondrial and plastid targeting sequences); SignalP (website: cbs.dtu.dk/services/SignalP/; predicts the presence and location of signal peptide cleavage sites in amino acid sequences from different organisms: Gram-positive prokaryotes, Gram-negative prokaryotes, and eukaryotes). The SignalP method incorporates a prediction of cleavage sites and a signal peptide/non-signal peptide prediction based on a combination of several artificial neural networks and hidden Markov models; and TargetP (website: cbs.dtu.dk/services/TargetP/) predicts the subcellular location of eukaryotic proteins, the location assignment being based on the predicted presence of any of the N-terminal presequences: chloroplast transit peptide (cTP), mitochondrial targeting peptide (mTP) or secretory pathway signal peptide (SP)). (See also, von Heijne, G., Eur J Biochem 133 (1) 17-21 (1983); Martoglio et al. Trends Cell Biol 8 (10):410-5 (1998); Hegde et al. Trends Biochem Sci 31(10):563-71 (2006); Dultz et al. J Biol Chem 283(15):9966-76 (2008); Emanuelsson et al. Nature Protocols 2(4) 953-971(2007); Zuegge et al. 280(1-2):19-26 (2001); Neuberger et al. J Mol Biol. 328(3):567-79 (2003); and Neuberger et al. J Mol Biol. 328(3):581-92 (2003)).

### Promoters

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Tissue-preferred promoters include those described by Van Ex et al., 2009, Plant Cell Rep. 28: 1509-1520; Yamamoto et al., 1997, Plant 12: 255-265; Kawamata et al., 1997, Plant Cell Physiol. 38: 792-803; Hansen et al., 1997, Mol. Gen. Genet. 254: 337-343; Russell et al., 1997, Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196; Orozco et al., 1993, Plant Mol. Biol. 23: 1129-1138; Matsuoka et al., 1993, Proc. Natl. Acad. Sci. USA 90: 9586-9590; and Guevara-Garcia et al., 1993, Plant J. 4: 495-505. Such promoters can be modified, if necessary, for weak expression.

Seed-specific promoters can be used to target gene expression to seeds in particular. Seed-specific promoters include promoters that are expressed in various tissues within seeds and at various stages of development of seeds. Seed-specific promoters can be absolutely specific to seeds, such that the promoters are only expressed in seeds, or can be expressed preferentially in seeds, e.g. at rates that are higher by 2-fold, 5-fold, 10-fold, or more, in seeds relative to one or more other tissues of a plant, e.g. stems, leaves, and/or roots, among other tissues. Seed-specific promoters include, for example, seed-specific promoters of dicots and seed-specific promoters of monocots, among others. For dicots, seed-specific promoters include, but are not limited to, bean β-phaseolin, napin, β-conglycinin, soybean oleosin 1, Arabidopsis thaliana sucrose synthase, flax conlinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, and globulin 1.

Exemplary promoters useful for expression of EDD and EDA proteins for specific dicot crops are disclosed in TABLE 8. Examples of promoters useful for increasing the expression of EDD and EDA proteins in specific monocot plants are disclosed in TABLE 9. For example, one or more of the promoters from soybean (Glycine max) listed in TABLE 8 may be used to drive the expression of one or more EDD and EDA genes encoding the proteins listed in TABLES 1-7.

TABLE 8
Promoters useful for expression of genes in dicots.

| | | Native | |
|---|---|---|---|
| | | organism | Gene ID* |
| Gene/Promoter | Expression | of promoter | (SEQ ID NO) |
| CaMV 35S | Constitutive | Cauliflower | (SEQ ID NO: 1) |
| | | mosaic virus | |
| Hsp70 | Constitutive | Glycine max | Glyma.02G093200 |
| | | | (SEQ ID NO: 2) |
| Chlorophyll A/B | Constitutive | Glycine max | Glyma.08G082900 |
| Binding Protein | | | (SEQ ID NO: 3) |
| (Cab5) | | | |
| Pyruvate phosphate | Constitutive | Glycine max | Glyma.06G252400 |
| dikinase (PPDK) | | | (SEQ ID NO: 4) |
| Actin | Constitutive | Glycine max | Glyma.19G147900 |
| | | | (SEQ ID NO: 5) |
| ADP-glucose | Seed-specific | Glycine max | Glyma.04G011900 |
| pyrophos-phorylase | | | (SEQ ID NO: 6) |
| (AGPase) | | | |
| Glutelin C (GluC) | Seed-specific | Glycine max | Glyma.03G163500 |
| | | | (SEQ ID NO: 7) |
| β-fructofuranosidase | Seed-specific | Glycine max | Glyma.17G227800 |
| insoluble isoenzyme | | | (SEQ ID NO: 8) |
| 1 (CIN1) | | | |
| MADS-Box | Cob-specific | Glycine max | Glyma.04G257100 |
| | | | (SEQ ID NO: 9) |
| Glycinin | Seed-specific | Glycine max | Glyma.03G163500 |
| (subunit G1) | | | (SEQ ID NO: 10) |
| oleosin isoform A | Seed-specific | Glycine max | Glyma.16G071800 |
| | | | (SEQ ID NO: 11) |
| Hsp70 | Constitutive | Brassica napus | BnaA09g05860D |
| Chlorophyll A/B | Constitutive | Brassica napus | BnaA04g20150D |
| Binding Protein | | | |
| (Cab5) | | | |
| Pyruvate phosphate | Constitutive | Brassica napus | BnaA01g18440D |
| dikinase (PPDK) | | | |
| Actin | Constitutive | Brassica napus | BnaA03g34950D |
| ADP-glucose | Seed-specific | Brassica napus | BnaA06g40730D |
| pyrophos-phorylase | | | |
| (AGPase) | | | |
| Glutelin C (GluC) | Seed-specific | Brassica napus | BnaA09g50780D |
| β-fructofuranosidase | Seed-specific | Brassica napus | BnaA04g05320D |
| insoluble isoenzyme | | | |
| 1 (CIN1) | | | |
| MADS-Box | Cob-specific | Brassica napus | BnaA05g02990D |
| Glycinin | Seed-specific | Brassica napus | BnaA01g08350D |
| (subunit G1) | | | |
| oleosin isoform A | Seed-specific | Brassica napus | BnaC06g12930D |
| 1.7S napin (nap A) | Seed-specific | Brassica napus | BnaA01g17200D |
| Sucrose synthase 2 | Seed-specific | Arabidopsis | SEQ ID NO: 151 |
| (SUS2) | | thaliana | |

*Gene ID includes sequence information for coding regions as well as associated promoters, 5′ UTRs, and 3′ UTRs and are available at Phytozome (see JGI website phytozome.jgi.doe.gov/pz/portal.html).

TABLE 9
Promoters useful for expression of genes in monocots, including maize and rice.

| Gene/Promoter | Expression | Rice* | Maize* | Other |
|---|---|---|---|---|
| Hsp70 | Constitutive | LOC_Os05g38530* | GRMZM2G310431* | |
| | | (SEQ ID NO: 12) | (SEQ ID NO: 20) | |
| Chlorophyll A/B | Constitutive | LOC_Os01g41710* | AC207722.2_FG009* | |
| Binding Protein | | (SEQ ID NO: 13) | (SEQ ID NO: 21) | |
| (Cab5) | | | GRMZM2G351977 | |
| | | | (SEQ ID NO: 22) | |
| maize ubiquitin | Constitutive | | (SEQ ID NO: 23) | |
| promoter/maize | | | | |
| ubiquitin intron | | | | |
| (sequence listed in | | | | |
| Genbank KT962835) | | | | |
| maize ubiquitin | Constitutive | | (SEQ ID NO: 24) | |
| promoter/maize | | | | |
| ubiquitin intron | | | | |
| (maize promoter and | | | | |
| intron sequence with | | | | |
| 99% identity to | | | | |
| sequence in Genbank | | | | |
| KT985051.1) | | | | |
| CaMV 35S | Constitutive | | | Cauliflower |
| | | | | mosaic virus |
| | | | | (SEQ ID NO: 11) |
| Pyruvate phosphate | Constitutive | LOC_Os05g33570* | GRMZM2G306345* | |
| dikinase (PPDK) | | (SEQ ID NO: 14) | (SEQ ID NO: 25) | |
| Actin | Constitutive | LOC_Os03g50885* | GRMZM2G047055* | |
| | | (SEQ ID NO: 15) | (SEQ ID NO: 26) | |
| Hybrid cab5/hsp70 | Constitutive | N/A | SEQ ID NO: 27 | |
| intron promoter | | | | |
| ADP-glucose | Seed-specific | LOC_Os01g44220* | GRMZM2G429899* | |
| pyrophos-phorylase | | (SEQ ID NO: 16) | (SEQ ID NO: 28) | |
| (AGPase) | | | | |
| Glutelin C (GluC) | Seed-specific | LOC_Os02g25640* | N/A | |
| | | (SEQ ID NO: 17) | | |
| β-fructofuranosidase | Seed-specific | LOC_Os02g33110* | GRMZM2G139300* | |
| insoluble isoenzyme 1 | | (SEQ ID NO: 18) | (SEQ ID NO: 29) | |
| (CIN1) | | | | |
| MADS-Box | Cob-specific | LOC_Os12g10540* | GRMZM2G160687* | |
| | | (SEQ ID NO: 19) | (SEQ ID NO: 30) | |
| Maize TrpA promoter | Seed-specific | | GRMZM5G841619 | |
| | | | (SEQIDNO: 31) | |

*Gene ID includes sequence information for coding regions as well as associated promoters, 5′ UTRs, and 3′ UTRs and are available at Phytozome (see JGI website phytozome.jgi.doe.gov/pz/portal.html).

Certain embodiments use genetically engineered plants or plant cells having multi-gene expression constructs harboring more than one transgene and promoter. The promoters can be the same or different.

Any of the described promoters can be used to control the expression of one or more of genes, their homologs and/or orthologs as well as any other genes of interest in a defined spatiotemporal manner.

Nucleic acid sequences intended for expression in genetically engineered plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

The coding sequence of the selected gene may be genetically engineered by altering the coding sequence for optimal expression in the crop species of interest. Methods for modifying coding sequences to achieve optimal expression in a particular crop species are well known (Perlak et al., 1991, Proc. Natl. Acad. Sci. USA 88: 3324 and Koziel et al., 1993, Biotechnology 11: 194-200).

Individual plants within a population of genetically engineered plants that express a recombinant gene(s) may have different levels of gene expression. The variable gene expression is due to multiple factors including multiple copies of the recombinant gene, chromatin effects, and gene suppression. Accordingly, a phenotype of the genetically engineered plant may be measured as a percentage of individual plants within a population. The yield of a plant can be measured simply by weighing. The yield of seed from a plant can also be determined by weighing. The increase in seed weight from a plant can be due to a number of factors, including an increase in the number or size of the seed pods, an increase in the number of seed and/or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A genetically engineered plant can be produced by selection of transformed seeds or by selection of transformed plant cells and subsequent regeneration.

In some embodiments, the genetically engineered plants are grown (e.g., on soil) and harvested. In some embodiments, above ground tissue is harvested separately from below ground tissue. Suitable above ground tissues include shoots, stems, leaves, flowers, grain, and seed. Exemplary below ground tissues include roots and root hairs. In some embodiments, whole plants are harvested and the above ground tissue is subsequently separated from the below ground tissue.

Genetic constructs may encode a selectable marker to enable selection of transformation events. There are many methods that have been described for the selection of transformed plants (for review see Miki et al., Journal of Biotechnology, 2004, 107, 193-232, and references incorporated therein). Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3′-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J. 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-458). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of genetically engineered plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296). Improved versions of many of the fluorescent proteins have been made for various applications. It will be apparent to those skilled in the art how to use the improved versions of these proteins, including combinations, for selection of transformants.

### Plastid Transformation

Alternatively, genes encoding the EDD and EDA enzymes can be inserted into and expressed directly from the plastid genome. Genetic constructs used for plastid-encoded transgene expression in a host organism typically comprise in the 5′-3′ direction, a left flank which mediates—together with the right flank—integration of the genetic construct into the target plastome; a promoter sequence; a sequence encoding a 5′ untranslated region (5′ UTR) containing a ribosome binding site; a sequence encoding a gene of interest, such as the genes disclosed herein; a 3′ untranslated region (3′ UTR); and a right flank. Plastid gene expression is regulated to a large extent at the post-transcriptional level and 5′ and 3′ UTRs have been shown to impact RNA stability and translation efficiency (Eibl et al., Plant J 19, 333-345 (1999)). Due to the prokaryotic nature of plastid expression systems, one or more transgenes may be arranged in an operon such that multiple genes are expressed from the same promoter. The promoter driving transcription of the operon may be located within the genetic construct, or alternatively, an endogenous promoter in the host plastome upstream of the transgene insertion site may drive transcription. In addition, the 3′UTR may be part of the right flank. The open reading frame may be orientated in either a sense or anti-sense direction. The construct may also comprise selectable marker gene(s) and other regulatory elements for expression.

Plastid-encoded expression can potentially yield high levels of expression due to the multiple copies of the plastome within a plastid and the presence of multiple plastids within the cell. Transgenic proteins have been observed to accumulate to 45% (De Cosa et al., Nat. Biotechnol. 19:71-74 (2001)) and >70% (Oey et al., Plant J. 57:436-445 (2009)) of the plant's total soluble protein. Since plastid DNA is maternally inherited in most plants, the presence of plastid-encoded transgenes in pollen is significantly reduced or eliminated, providing some level of gene containment in plants created by plastid transformation.

The plants modified for enhanced yield may have stacked input traits that include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162).

### EXAMPLES

### Example 1. Improvement of Carbon Conversion Efficiency in Camelina Seed

The Camelina sativa oilseed has a poor carbon conversion efficiency (CCE; moles of carbon in biomass per mole of carbon in phloem-supplied substrates), and the reason for this has recently been determined to be a high flux through the oxidative pentose phosphate pathway (Carey et al., 2020, Plant Physiol. 182:493-506). Under conditions mimicking physiological light (10 μmol m⁻² s⁻¹ reaching the seed), C. sativa embryos in culture showed a carbon conversion efficiency of around 30%, compared to the closely related Brassica napus, whose embryos under similar conditions had a carbon conversion efficiency of around 80% (Goffman et al., 2005, Plant Physiol. 138:2269-2279). The OPP pathway is largely the reverse of the carbon-fixing Calvin cycle, and thus it produces rather than consumes CO₂. Metabolic flux labeling experiments have shown that canola uses Rubisco, without employing the Calvin cycle, to recapture CO₂ released in the seed increasing its carbon conversion efficiency (FIG. 2) (Schwender et al., Nature, 432, 779 (2004)). Similar metabolic flux labeling experiments in Camelina have shown that it does not recapture CO₂ in the seed with Rubisco, likely leading to its decreased carbon conversion efficiency compared to canola (Carey et al., 2020). When illuminated, embryonic cultures of C. sativa do not induce carbon fixation pathways associated with Rubisco; instead a subtle shift from the OPP pathway to the Embden-Meyerhof-Parnas (glycolytic) pathway (FIG. 1) begins to occur, and neither the reasons nor the mechanisms for this are clear (Carey et al., 2020, Plant Physiol. 182:493-506). Furthermore, while Arabidopsis thaliana, a species closely related to C. sativa, has plentiful Rubisco expression in its siliques (Arabidopsis eFP Browser 2.0, website: bar.utoronto.ca/efp2/Arabidopsis/Arabidopsis_eFPBrowser2.html), C. sativa has very little, especially as the seed matures (Camelina eFP Browser, website: bar.utoronto.ca/efp_camelina/cgi-bin/efpWeb.cgi).

In order to improve the carbon conversion efficiency of the camelina seed, the OPP flux can be displaced partially or entirely by one or more alternative pathways: the Embden-Meyerhof-Parnas (glycolytic) pathway, the Entner-Doudoroff (ED) pathway, and/or the use of Rubisco without a Calvin cycle. FIG. 1 shows the ED, OPP, and glycolytic pathways, while FIG. 2 shows the pathway utilizing Rubisco without the Calvin cycle.

In the present work, flux-balance analysis was performed by using one or more of the Rubisco, ED, and/or glycolytic pathways, to displace carbon flux through the OPP pathway. This analysis showed that the maximum theoretical carbon conversion efficiency for a C. sativa seed, even in the absence of incident light, is nearly 80% and increases with illumination (TABLE 10).

TABLE 10
Maximum carbon conversion efficiency (CCE) of camelina
seed subject to various metabolic constraints.

| Permitted pathways | | | | |
|---|---|---|---|---|
| Rubisco | ED | Glycolysis | Light* | Maximum CCE (%) |
| + | + | + | 10 | 79.5 |
| + | − | − | 10 | 74.4 |
| − | + | − | 10 | 77.3 |
| − | − | + | 10 | 79.2 |
| + | + | + | 0 | 77.3 |
| + | − | − | 0 | 72.2 |
| − | + | − | 0 | 75.1 |
| − | − | + | 0 | 77.0 |
| − | − | − | 10 | 26.7 |

*Total photons reaching seed biomass per 1000 photons reaching leaf biomass. Flux balance analysis performed using a modified AraGEM model (C. G. de Oliveira Dal'Molin et al., 2010, 152, 579) as the stoichiometric framework.

Flux-balance analysis, when the OPP pathway is forced to act at a level similar to that reported in Carey et al., 2020, Plant Physiol. 182:493-506, calculates a maximum carbon conversion efficiency of about 30% at physiological light and a positive correlation with light intensity, agreeing well with the data reported by Carey et al. (2020). Any of the three alternative routes of sugar dissimilation shown here are thus capable of providing a major improvement over the observed carbon conversion efficiency values when the OPP pathway is dominant. While the OPP pathway should not be eliminated entirely within a plant because of the role of its component enzymes in producing ribose-5-phosphate for nucleotide synthesis and also as a source of supplementary NADPH, especially when there is no incident light, there are several potential ways to reduce the impact of an overactive OPP pathway, which are described in the following examples.

### Example 2. Seed-Specific Expression of the Entner-Doudoroff Pathway in Camelina Oilseed

The simplest and most straightforward approach of the three options presented in Example 1 for improvement of carbon conversion efficiency in camelina seed is to increase expression of the Entner-Doudoroff pathway, for the following reasons:

1) The C. sativa seed already has a high carbon flux from glucose-6-phosphate to 6-phosphogluconate via glucose-6-phosphate dehydrogenase (ZWF; Carey, 2020), and thus no fundamental restructuring of carbon flux is necessary.

2) Overexpression of only two genes, those encoding the EDD (6-phosphogluconate dehydratase, EC 4.2.1.12) and EDA (KDPG aldolase, EC 4.1.2.14) proteins, is required to derive benefits.

3) Neither EDD nor EDA has any cofactor requirements.

4) EDD and EDA are not likely to be subject to any coherent post-transcriptional regulation in the plant cell if they are imported from another source.

5) The ED pathway produces the fewest energy carriers per glucose consumed, meaning it can potentially operate the most quickly and provide the strongest sink-strength benefit.

6) The ED pathway is thermodynamically favorable overall, as are the EDD and EDA reactions individually.

EDD and EDA genes from different organisms can be synthesized and cloned into genetic constructs for transient expression in protoplasts isolated from leaves of plants such as Camelina or Arabidopsis. Assays for enzyme activity of EDD and EDA are known in the art (e.g., Conway et al., 1991, Mol. Microbiol. 5:2901-2911; Zlabotny et al., 1967, J. Bacteriol. 93:1579-1581). Protoplasts can be pelleted by centrifugation after transient expression of the proteins, the cells can be lysed, and enzyme assays performed. Genes encoding the proteins listed in TABLES 1-7 can be tested in protoplasts and enzymes with optimal activity can be used for producing plant transformation constructs.

### Example 3. Seed-Specific Expression of Plastid-Targeted EDD and EDA in Camelina sativa

The simplest way to engineer the ED pathway in seed tissue is to express the genes encoding EDD and EDA, each with either an endogenous or an appended plastid targeting signal, under the control of a seed-specific promoter. This will direct the EDD and EDA proteins to seed plastids. It may not be necessary to express GDH and GCK (FIG. 1), as 6-phosphogluconate is already produced by the plastidic ZWF protein of the OPP pathway, which is dominant in camelina seeds (Carey et al., 2020, Plant Physiol. 182:493-506). However, additional expression of GDH and/or GCK could encourage glucose to be catabolized by the ED pathway instead of the EMP pathway. Cytosolic expression of EDD and EDA could also be beneficial, as plants like A. thaliana have at least some cytosolic ZWF activity (Wakao et al., 2008, Plant Physiol. 146:277-288).

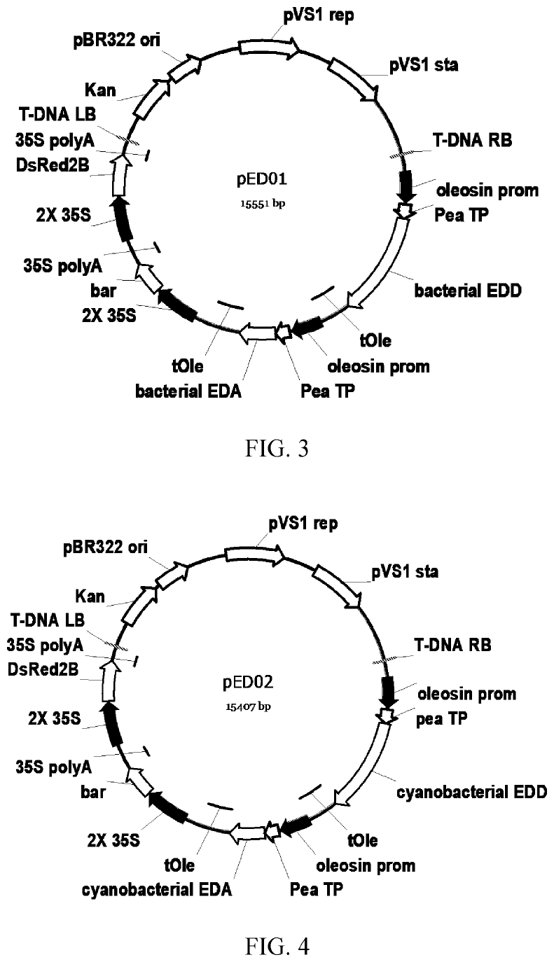

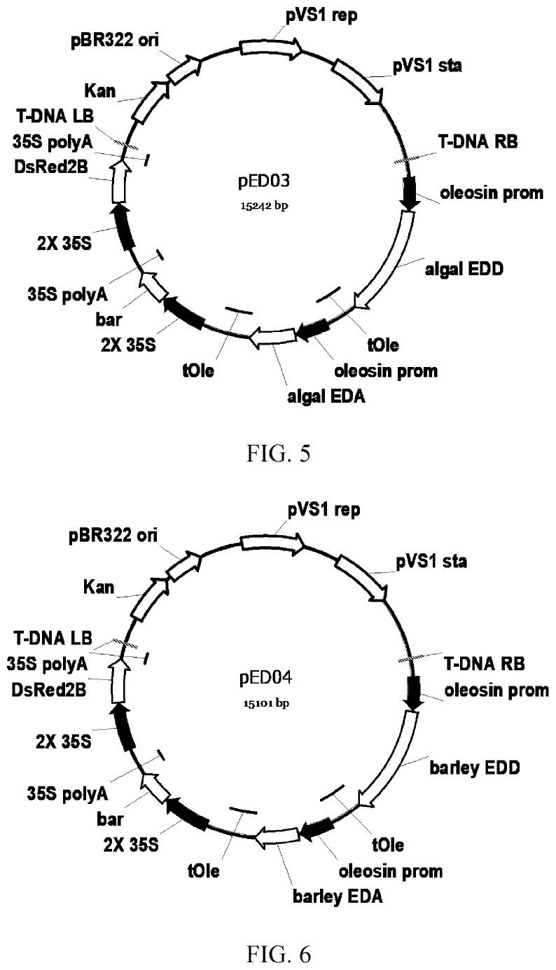

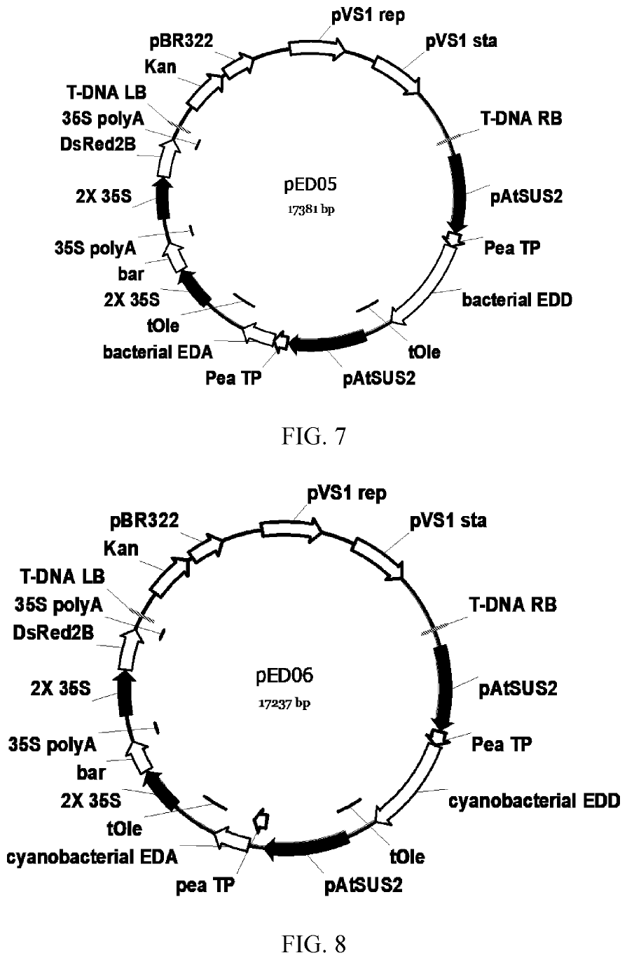

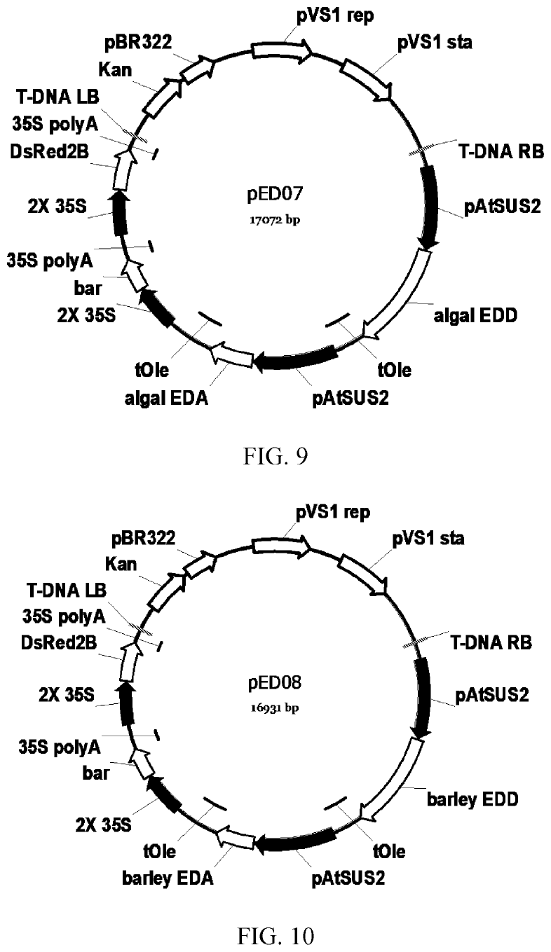

To target the EDD and EDA proteins to the plastids of seeds, several transformation vectors were designed that use EDD and EDA from different sources, including the bacterium Zymomonas mobilis (FIGS. 3 and 7), the cyanobacterium Synechocystis sp. PCC 6803 (FIGS. 4 and 8), the algae Guillardia theta and Phaeodactylum tricornutum (FIGS. 5 and 9), and Hordeum vulgare (barley, FIGS. 6 and 10). Each gene cassette for the bacterial or cyanobacterial EDD or EDA was designed containing DNA encoding an N-terminal plastid targeting signal fused to the 5′ end of the gene. Algal and plant EDD genes and plant EDA genes likely have endogenous plastid targeting signals and thus an N-terminal targeting signal was not added. However constructs with algal or plant EDD and/or EDA can optionally have an N-terminal plastid targeting signal fused to the 5′ end of the protein(s) if correct transport does not take place. The N-terminal plastid targeting signal was designed to contain DNA encoding the signal peptide of the small subunit of Rubisco from pea and the first 24 amino acids of the mature protein (Cashmore, 1983) DNA encoding a two-amino-acid linker that contains an XbaI restriction site allowed fusion of the desired transgene to the targeting sequence (Kourtz et al., Plant Biotechnology Journal, 3, 435 (2005). For the bacterial or cyanobacterial EDD and EDA sequences, the ATG start site of the gene encoding the EDD and EDA proteins was changed to a GTG. Genes were designed to be expressed from either the soybean oleosin promoter (SEQ ID NO: 11) (FIGS. 3-6) or the SUS2 promoter from Arabidopsis thaliana (SEQ ID NO: 151) (FIGS. 7-10).

A construct selected from pED01 (SEQ ID NO: 152), pED02 (SEQ ID NO: 153), pED03 (SEQ ID NO: 154), pED04 (SEQ ID NO: 155), pED05 (SEQ ID NO: 156), pED06 (SEQ ID NO: 157), pED07 (SEQ ID NO: 158), and pED08 (SEQ ID NO: 159) can be prepared and transformed into Camelina sativa cv CS0043 (abbreviated as WT43) using a floral dip procedure as follows.

In preparation for plant transformation experiments, seeds of Camelina sativa germplasm 10CS0043 (abbreviated WT43, obtained from Agriculture and Agri-Food Canada) are sown directly into 4 inch (10 cm) pots filled with soil in the greenhouse. Growth conditions are maintained at 24° C. during the day and 18° C. during the night. Plants are grown until flowering. Plants with a number of unopened flower buds are used in ‘floral dip’ transformations.

Agrobacterium strain GV3101 (pMP90) is transformed with the genetic construct of interest using electroporation. A single colony of GV3101 (pMP90) containing the genetic construct of interest is obtained from a freshly streaked plate and is inoculated into 5 mL LB medium. After overnight growth at 28° C., 2 mL of culture is transferred to a 500-mL flask containing 300 mL of LB and incubated overnight at 28° C. Cells are pelleted by centrifugation (6,000 rpm, 20 min), and diluted to an OD600 of ˜0.8 with infiltration medium containing 5% sucrose and 0.05% (v/v) Silwet-L77 (Lehle Seeds, Round Rock, Tex., USA). Camelina plants are transformed by “floral dip” using the transformation construct as follows. Pots containing plants at the flowering stage were placed inside a 460 mm height vacuum desiccator (Bel-Art, Pequannock, N.J., USA). Inflorescences are immersed into the Agrobacterium inoculum contained in a 500-ml beaker. A vacuum (85 kPa) is applied and held for 5 min. Plants are removed from the desiccator and are covered with plastic bags in the dark for 24 h at room temperature. Plants are removed from the bags and returned to normal growth conditions within the greenhouse for seed formation (T1 generation of seed).

T1 seeds are obtained and screened for the expression of the visual marker DsRed, a marker on the T-DNA in the plasmid vectors (FIGS. 3-10). Independent transgenic events are identified. The Dsred positive T1 lines are grown in the greenhouse along with the wild type controls. Agronomic and yield evaluation of multiple plants is performed in the T2 generation on single copy and multiple copy lines. T3 seed is collected and seed yield and oil content is determined. The oil content of T3 seeds is measured using published procedures for preparation of fatty acid methyl esters (Malik et al. 2015, Plant Biotechnology Journal, 13, 675-688).

### Example 4. Use of Entner-Doudoroff Pathway to Increase Seed Sink Strength in Plants

In many crops, the factor that limits yield under nutrient-sufficient conditions is sink strength, or the rate at which phloem-supplied carbohydrates and amino acids are consumed by the seed. Often the overall metabolism of the seed produces more NAD(P)H and/or ATP than is actually required for the production of seed biomass. Some of the metabolic pathways responsible for producing these cofactors could be made more thermodynamically favorable, and thus likely faster, if they could ultimately accomplish the same conversion of substrate to product without reducing NAD(P)⁺ or phosphorylating ADP. This type of strategy can be employed up to a certain point in a seed without reducing its carbon conversion efficiency (CCE; moles of carbon in biomass per mole of carbon in phloem-supplied substrates), yet speeding up the overall metabolism to deplete phloem substrates more quickly and thus increasing demand for photosynthate. Many crops will respond to this increased demand for photosynthate by increasing their rate of photosynthesis, as there is generally substantially more capacity to produce photosynthate from CO₂ than the capacity to metabolize the photosynthate downstream in sink tissues such as the seed. The Entner-Doudoroff pathway is an example of a metabolic modification in the seed that could provide these advantages to seed yield.

The sink-strength benefit of the Entner-Doudoroff pathway is its limited production of NAD(P)H and ATP when compared to the other two traditional sugar-catabolism pathways. For each glucose consumed, the EMP pathway (FIG. 1) has a net production of 2 NADH, 2 ATP and 2 pyruvate; the OPP pathway (FIG. 1) has a net production of 2 NADPH, 1.67 NADH, 1.67 ATP, and 1.67 pyruvate; and the ED pathway (FIG. 1) has a net production of 1 NADPH, 1 NADH, 1 ATP, and 2 pyruvate. Therefore the ED pathway has the lowest overall production of ATP and NAD(P)H and is therefore likely to be accomplished more quickly than the others overall. In addition, the ED pathway has the fewest individual reactions per glucose consumed that produce either NAD(P)H or ATP, making it the least likely to encounter kinetic difficulties due to cofactor imbalances.

### Example 5. Seed Specific Expression of Plastid-Targeted EDD and EDA in Canola

To increase seed sink strength in canola, it can be transformed with a construct selected from pED01 (SEQ ID NO: 152), pED02 (SEQ ID NO: 153), pED03 (SEQ ID NO: 154), pED04 (SEQ ID NO: 155), pED05 (SEQ ID NO: 156), pED06 (SEQ ID NO: 157), pED07 (SEQ ID NO: 158), and pED08 (SEQ ID NO: 159) (FIGS. 3-10) expressing the plastid targeted EDD and EDA proteins as follows.

In preparation for plant transformation experiments, seeds of Brassica napus cv DH12075 (obtained from Agriculture and Agri-Food Canada) are surface sterilized with sufficient 95% ethanol for 15 seconds, followed by 15 minutes incubation with occasional agitation in full strength Javex (or other commercial bleach, 7.4% sodium hypochlorite) and a drop of wetting agent such as Tween 20. The Javex solution is decanted and 0.025% mercuric chloride with a drop of Tween 20 is added and the seeds are sterilized for another 10 minutes. The seeds are then rinsed three times with sterile distilled water. The sterilized seeds are plated on half strength hormone-free Murashige and Skoog (MS) media (Murashige T, Skoog F (1962). Physiol Plant 15:473-498) with 1% sucrose in 15×60 mm petri dishes that were then placed, with the lid removed, into a larger sterile vessel (Majenta GA7 jars). The cultures are kept at 25° C., with 16 h light/8h dark, under approximately 70-80 μE of light intensity in a tissue culture cabinet. 4-5 days old seedlings are used to excise fully unfolded cotyledons along with a small segment of the petiole. Excisions are made so as to ensure that no part of the apical meristem is included.

Agrobacterium strain GV3101 (pMP90) carrying the genetic construct of interest is grown overnight in 5 ml of LB media with 50 mg/L kanamycin, gentamycin, and rifampicin. The culture is centrifuged at 2000 g for 10 min., the supernatant is discarded and the pellet is suspended in 5 ml of inoculation medium (Murashige and Skoog with B5 vitamins [MS/B5; Gamborg O L, Miller R A, Ojima K. Exp Cell Res 50:151-158], 3% sucrose, 0.5 mg/L benzyl aminopurine (BA), pH 5.8). Cotyledons are collected in Petri dishes with ˜1 ml of sterile water to keep them from wilting. The water is removed prior to inoculation and explants are inoculated in a mixture of 1 part Agrobacterium suspension and 9 parts inoculation medium in a final volume sufficient to bathe the explants. After explants are well exposed to the Agrobacterium solution and inoculated, a pipet is used to remove any extra liquid from the petri dishes.

The Petri plates containing the explants incubated in the inoculation media are sealed and kept in the dark in a tissue culture cabinet set at 25° C. After 2 days the cultures are transferred to 4° C. and incubated in the dark for 3 days. The cotyledons, in batches of 10, are then transferred to selection medium consisting of Murashige Minimal Organics (Sigma), 3% sucrose, 4.5 mg/L BA, 500 mg/L IVIES, 27.8 mg/L Iron (II) sulfate heptahydrate, pH 5.8, 0.7% Phytagel with 300 mg/L timentin, and 2 mg/L L-phosphinothricin (L-PPT) added after autoclaving. The cultures are kept in a tissue culture cabinet set at 25° C., 16 h/8 h, with a light intensity of about 125 μmol m⁻² s⁻¹. The cotyledons are transferred to fresh selection every 3 weeks until shoots are obtained. The shoots are excised and transferred to shoot elongation media containing MS/B5 media, 2% sucrose, 0.5 mg/L BA, 0.03 mg/L gibberellic acid (GA₃), 500 mg/L 4-morpholineethanesulfonic acid (MES), 150 mg/L phloroglucinol, pH 5.8, 0.9% Phytagar and 300 mg/L timentin and 3 mg/L L-phosphinothricin added after autoclaving. After 3-4 weeks any callus that formed at the base of shoots with normal morphology is cut off and shoots are transferred to rooting media containing half strength MS/B5 media with 1% sucrose and 0.5 mg/L indole butyric acid, 500 mg/L MES, pH 5.8, 0.8% agar, with 1.5 mg/L L-PPT and 300 mg/L timentin added after autoclaving. The plantlets with healthy shoots were hardened and transferred to 6 inch (15 cm) pots in the greenhouse. 148 T0 lines transformed with the genetic construct of interest are generated and are grown in the greenhouse. Single copy lines are identified. Plants are allowed to grow in the greenhouse and produce T1 transgenic seeds, which are then collected.

Screening of transgenic plants of canola expressing the EDD and EDA proteins from the genetic construct of interest to identify plants with higher yield and/or oil content is performed as follows. The T1 seeds of several independent lines are grown in a randomized complete block design in a greenhouse maintained at 24° C. during the day and 18° C. during the night. The T2 generation of seed from each line is harvested. Seed yield from each plant is determined by harvesting all of the mature seeds from a plant and drying them in an oven with mechanical convection set at 22° C. for two days. The weight of the entire harvested seed is recorded. The 100 seed weight is measured to obtain an indication of seed size. The oil content of seeds is measured using published procedures for preparation of fatty acid methyl esters (Malik et al. 2015, Plant Biotechnology Journal, 13, 675-688).

### Example 6. Seed Specific Expression of Plastid-Targeted EDD, EDA, ZWF, and/or PGL in Camelina and Canola

Plants generally have the means to produce 6-phosphogluconate via the action of the glucose-6-phosphate 1-dehydrogenase (ZWF; EC 1.1.1.49) and optionally 6-phosphogluconolactonase (PGL; EC 3.1.1.31) enzymes (FIG. 1). Therefore, the minimum requirement for implementation of the ED pathway in plants is overexpression in the seed plastid of the genes encoding the EDD and EDA enzymes. However, not all plants will utilize ZWF and PGL to the same extent under physiological conditions, and therefore it may be advantageous in some plants to overexpress the genes encoding these proteins in the seed plastid as well.

ZWF- and PGL-encoding genes can be isolated from many different organisms for expression in plants. One can overexpress the endogenous genes in the crop of interest, with its endogenous plastid-targeting signal, or to avoid the regulatory systems of the plant, which may inhibit the activity of ZWF and/or PGL, one can choose these genes from bacteria or algae. For bacterial genes, a plastid-targeting signal can be fused to the N-terminus of the genes encoding ZWF and PGL to direct the genes to the plastid. TABLE 11 and TABLE 12 give representative examples of bacterial ZWF and PGL proteins, respectively. Genes encoding ZWF and PGL are found in numerous bacteria, and thus TABLE 11 and TABLE 12 only list preferred sources of these genes, such as bacteria used in food preparation.

TABLE 11
Glucose-6-phosphate 1-dehydrogenase
(ZWF) proteins from benign bacteria.

| | GenBank | SEQ |
|---|---|---|
| Species | accession | ID NO. |
| Acetobacter aceti | AQS86425 | 141 |
| Hafnia alvei | AIU72650 | 142 |
| Proteus vulgaris | ATN00454 | 143 |
| Pseudomonas fluorescens | ABA74328 | 144 |
| Zymomonas mobilis | AAV88991 | 145 |

TABLE 12
6-Phosphogluconolactonase (PGL) proteins from benign bacteria.

| | GenBank | SEQ |
|---|---|---|
| Species | accession | ID NO. |
| Acetobacter aceti | AQS86447 | 146 |
| Hafnia alvei | AIU72651 | 147 |
| Proteus vulgaris | ATN01314 | 148 |
| Pseudomonas fluorescens | ABA76099 | 149 |
| Zymomonas mobilis | AAV90102 | 150 |

For this purpose, genetic constructs pED01 (SEQ ID NO: 152), pED02 (SEQ ID NO: 153), pED03 (SEQ ID NO: 154), pED04 (SEQ ID NO: 155), pED05 (SEQ ID NO: 156), pED06 (SEQ ID NO: 157), pED07 (SEQ ID NO: 158), and pED08 (SEQ ID NO: 159) (FIGS. 3-10) can be modified to include expression cassettes for plastid targeted ZWF (also termed pt-ZWF) and plastid targeted PGL (also termed pt-PGL). Example expression constructs for these four genes are shown in TABLE 13. These modified constructs can be transformed into Camelina and/or canola using the procedures described above. While the example in TABLE 13 uses the soybean oleosin promoter to express each gene, it will be apparent to those skilled in the art that many different combinations of promoters can be used to practice the invention.

TABLE 13
Transformation cassettes for seed-specific expression of
genes encoding the pt-EDD and pt-EDA with seed specific
expression of genes encoding pt-ZWF and pt-PGL in oilseeds

| | | |
|---|---|---|
| Promoter | Gene* | Terminator |

Expression Cassette 1: pt-EDD expression cassette

| | | |
|---|---|---|
| Soybean oleosin promoter | Pt-EDD gene from | 3′ UTR from the |
| (SEQ ID NO: 11) | Zymomonas mobilis | soybean oleosin gene |
| | (SEQ ID NO: 160) | (SEQ ID NO: 164) |

Expression Cassette 2: pt-EDA expression cassette

| | | |
|---|---|---|
| Soybean oleosin promoter | Pt-EDA gene from | 3′ UTR from the |
| (SEQ ID NO: 11) | Zymomonas mobilis | soybean oleosin gene |
| | (SEQ ID NO: 161) | (SEQ ID NO: 164) |

Expression Cassette 3: pt-ZWF expression cassette

| | | |
|---|---|---|
| Soybean oleosin promoter | Pt-ZWF gene from | 3′ UTR from the |
| (SEQ ID NO: 11) | Zymomonas mobilis | soybean oleosin gene |
| | (SEQ ID NO: 162) | (SEQ ID NO: 164) |

Expression Cassette 4: pt-PGL expression cassette

| | | |
|---|---|---|
| Soybean oleosin promoter | Pt-PGL gene from | 3′ UTR from the |
| (SEQ ID NO: 11) | Zymomonas mobilis | soybean oleosin gene |
| | (SEQ ID NO: 163) | (SEQ ID NO: 164) |

*pt-EDD, pt-EDA, pt-ZWF and pt-PGL indicate genes encoding EDD, EDA, ZWF and PGL with an added plastid targeting signal at the N-terminus. The plastid targeting signal is from the small subunit of Rubisco from pea (Cashmore, 1983).

### Example 7. Seed Specific Expression of Plastid-Targeted EDD, EDA, ZWF, and/or PGL in Maize

Expression cassettes for the a gene encoding plastid-targeted EDD (pt-EDD) and an expression cassette for the gene encoding plastid-targeted EDA (pt-EDA) can be constructed using a variety of different promoters for expression in maize. Candidate constitutive and seed-specific promoters for use in monocots including corn are listed in TABLE 9, however those skilled in the art will understand that other promoters can be selected for expression.

In some instances, it may be advantageous to create a hybrid promoter containing a promoter sequence and an intron. These promoters can deliver higher levels of stable expression. Examples of such hybrid promoters include the hybrid maize Cab-m5 promoter/maize hsp70 intron (SEQ ID NO: 27, TABLE 9) and the maize ubiquitin promoter/maize ubiquitin intron (SEQ ID NO: 23 and 24, TABLE 9).

Example expression cassettes for seed specific expression of genes encoding plastid targeted EDD and EDA in maize include the genetic elements in TABLE 14 (Expression Cassette 1), in which the maize trpA promoter is operably linked to the gene encoding pt-EDD which is operably linked to the termination sequence. Expression cassette 2 contains the trpA promoter operably linked to the gene encoding pt-EDA which is operably linked to a termination sequence. An additional expression cassette containing a selectable marker, such as the bar gene driven by the maize ubiquitin promoter/maize ubiquitin intron, can be used to confer glufosinate tolerance or bialophos resistance for selection of transformants. These expression cassettes can be transformed into maize protoplasts, calli, or immature embryos using biolistics as reviewed in Que et al., 2014, either by delivery on a single DNA fragment or co-transformation of two DNA fragments.

Example expression cassettes for a pathway encompassing EDD, EDA, ZWF, and PGL include the expression cassettes listed in TABLE 15.

The expression cassettes in TABLES 14 and 15 use the trpA seed specific promoter for each transgene expression cassette. It may be advantageous to use a different promoter, or different combinations of promoters, for expression of the pt-EDD, pt-EDA, pt-ZWF, and pt-PGL genes, to increase stability of the binary vector or transgene insert, or to prevent silencing of transgenes.

TABLE 14
Transformation cassettes for seed specific expression
of genes encoding the pt-EDD and pt-EDA genes in maize

| | | |
|---|---|---|
| Promoter | Gene* | Terminator |

Expression Cassette 1: pt-EDD expression cassette

| | | |
|---|---|---|
| Maize trpA promoter | Pt-EDD gene from | Maize trpA 3′ UTR |
| (SEQ ID NO: 31) | Zymomonas mobilis | |
| | (SEQ ID NO: 160) | |

Expression Cassette 2: pt-EDA expression cassette

| | | |
|---|---|---|
| Maize trpA promoter | Pt-EDA gene from | Maize trpA 3′ UTR |
| (SEQ ID NO: 31) | Zymomonas mobilis | |
| | (SEQ ID NO: 161) | |

*pt-EDD and pt-EDA indicate genes encoding EDD and EDA with an added plastid targeting signal at the N-terminus. The plastid targeting signal is from the small subunit of Rubisco from pea (Cashmore, 1983).

TABLE 15
Transformation cassettes for seed-specific expression of
genes encoding the pt-EDD and pt-EDA with seed specific
expression of genes encoding pt-ZWF and pt-PGL in maize

| | | |
|---|---|---|
| Promoter | Gene* | Terminator |

Expression Cassette 1: pt-EDD expression cassette

| | | |
|---|---|---|
| Maize trpA promoter | Pt-EDD gene from | Maize trpA 3′ UTR |
| (SEQ ID NO: 31) | Zymomonas mobilis | |
| | (SEQ ID NO: 160) | |

Expression Cassette 2: pt-EDA expression cassette

| | | |
|---|---|---|
| Maize trpA promoter | Pt-EDA gene from | Maize trpA 3′ UTR |
| (SEQ ID NO: 31) | Zymomonas mobilis | |
| | (SEQ ID NO: 161) | |

Expression Cassette 3: pt-ZWF expression cassette

| | | |
|---|---|---|
| Maize trpA promoter | Pt-ZWF gene from | Maize trpA 3′ UTR |
| (SEQ ID NO: 31) | Zymomonas mobilis | |
| | (SEQ ID NO: 162) | |

Expression Cassette 4: pt-PGL expression cassette

| | | |
|---|---|---|
| Maize trpA promoter | Pt-PGL gene from | Maize trpA 3′ UTR |
| (SEQ ID NO: 31) | Zymomonas mobilis | |
| | (SEQ ID NO: 163) | |

*pt-EDD, pt-EDA, pt-ZWF and pt-PGL indicate genes encoding EDD, EDA, ZWF and PGL with an added plastid targeting signal at the N-terminus. The plastid targeting signal is from the small subunit of Rubisco from pea (Cashmore, 1983).

It will be apparent to those skilled in the art that many selectable markers can be used in maize transformations for the expression cassettes described in TABLES 14 and 15 that are not derived from plant pest sequences for selection purposes. These include maize acetolactate synthase/acetohydroxy acid synthase (ALS/AHAS) mutant genes conferring resistance to a range of herbicides from the ALS family of herbicides, including chlorsulfuron and imazethapyr; a 5-enolpyruvoylshikimate-3-phosphate synthase (EPSPS) mutant gene from maize, providing resistance to glyphosate; as well as multiple other selectable markers that are all reviewed in Que et al., 2014 (Que, Q. et al., Front. Plant Sci. 5 Aug. 2014; doi.org/10.3389/fpls.2014.00379).

Methods to transform the expression cassette described in TABLES 14 and 15 into maize are routine and well known in the art and have recently been reviewed by Que et al., (2014), Frontiers in Plant Science 5, article 379, pp 1-19.

Protoplast transformation methods useful for practicing the invention are well known to those skilled in the art. Such procedures include for example the transformation of maize protoplasts as described by Rhodes and Gray (Rhodes, C. A. and D. W. Gray, Transformation and regeneration of maize protoplasts, in Plant Tissue Culture Manual: Supplement 7, K. Lindsey, Editor. 1997, Springer Netherlands: Dordrecht. p. 353-365). For protoplast transformation of maize, the expression cassettes described in TABLES 14 and 15 can be co-bombarded, or delivered on a single DNA fragment. The bar gene imparting transgenic plants resistance to bialophos is used for selection.

For Agrobacterium-mediated transformation of maize, the expression cassettes described in TABLES 14 and 15 can be inserted into a binary vector. The binary vector is transformed into an Agrobacterium tumefaciens strain, such as A. tumefaciens strain EHA101. Agrobacterium-mediated transformation of maize can be performed following a previously described procedure (Frame et al. (2006), Agrobacterium Protocols, Wang K., ed., Vol. 1, pp 185-199, Humana Press) as follows.

Plant Material: Plants grown in a greenhouse are used as an explant source. Ears are harvested 9-13 days after pollination and surface sterilized with 80% ethanol.

Explant Isolation, Infection and Co-Cultivation: Immature zygotic embryos (1.2-2.0 mm) are aseptically dissected from individual kernels and incubated in an A. tumefaciens strain EHA101 culture containing the transformation vector (grown in 5 ml N6 medium supplemented with 100 μM acetosyringone for stimulation of the bacterial vir genes for 2-5 h prior to transformation) at room temperature for 5 min. The infected embryos are transferred scutellum side up on to a co-cultivation medium (N6 agar-solidified medium containing 300 mg/l cysteine, 5 μM silver nitrate and 100 μM acetosyringone) and incubated at 20° C., in the dark for 3 d. Embryos are transferred to N6 resting medium containing 100 mg/l cefotaxime, 100 mg/l vancomycin and 5 μM silver nitrate and incubated at 28° C., in the dark for 7 d.

Callus Selection: All embryos are transferred on to the first selection medium (the resting medium described above supplemented with 1.5 mg/l bialaphos) and incubated at 28° C. in the dark for 2 weeks followed by subculture on a selection medium containing 3 mg/l bialaphos. Proliferating pieces of callus are propagated and maintained by subculture on the same medium every 2 weeks.

Plant Regeneration and Selection: Bialaphos-resistant embryogenic callus lines are transferred on to regeneration medium I (MS basal medium supplemented with 60 g/l sucrose, 1.5 mg/l bialaphos and 100 mg/l cefotaxime and solidified with 3 g/l Gelrite) and incubated at 25° C. in the dark for 2 to 3 weeks. Mature embryos formed during this period are transferred on to regeneration medium II (the same as regeneration medium I with 3 mg/l bialaphos) for germination in the light (25° C., 80-100 μmol/m²/s light intensity, 16/8-h photoperiod). Regenerated plants are ready for transfer to soil within 10-14 days. Plants are grown in the greenhouse to maturity and T1 seeds are isolated.

The copy number of the transgene insert is determined, through methods such as Southern blotting or digital PCR, and lines are selected to bring forward for further analysis. Overexpression of the genes encoding pt-EDD, pt-EDA, pt-ZWF, and/or pt-PGL are determined by RT-PCR and/or Western blotting techniques and plants with the desired level of expression are selected. Homozygous lines are generated. The yield seed of homozygous lines is compared to control lines.

### Example 8. Seed Specific Expression of Pt-EDD, Pt-EDA, Pt-ZWF, and/or Pt-PGL in Soybean

For seed specific expression of the pt-EDD and pt-EDA genes in soybean, the expression cassettes described in TABLE 16 are constructed using cloning techniques standard for those skilled in the art. For seed specific expression of the pt-EDD, pt-EDA, pt-ZWF, and/or pt-PGL genes in soybean, the expression cassettes described in TABLE 17 are constructed. The expression cassettes in TABLES 16 and 17 use the seed-specific promoter from the soya bean oleosin isoform A gene (SEQ ID NO: 11) for each transgene expression cassette. It may be advantageous to use a different promoter for expression of the pt-EDD, pt-EDA, pt-ZWF, and/or pt-PGL genes, to increase stability of the binary vector or transgene insert, or to prevent silencing of transgenes. It will be apparent to those skilled in the art that many different promoters are available for expression in plants. TABLE 8 lists additional options for use in dicots that can be used as alternate promoters for expression cassettes described in TABLES 16 and 17.

TABLE 16
Transformation cassettes for seed specific expression
of the pt-EDD and pt-EDA genes in soybean

| | | |
|---|---|---|
| Promoter | Gene* | Terminator |

Expression Cassette 1: pt-EDD expression cassette

| | | |
|---|---|---|
| seed-specific promoter | pt-EDD gene from | Terminator from the |
| from the soya bean | Zymomonas mobilis | soya bean oleosin |
| oleosin isoform A gene | (SEQ ID NO: 160) | isoform A gene |
| (SEQ ID NO: 11) | | (SEQ ID NO: 164) |

Expression Cassette 2: pt-EDA expression cassette

| | | |
|---|---|---|
| seed-specific promoter | pt-EDA gene from | Terminator from the |
| from the soya bean | Zymomonas mobilis | soya bean oleosin |
| oleosin isoform A gene | (SEQ ID NO: 161) | isoform A gene |
| (SEQ ID NO: 11) | | (SEQ ID NO: 164) |

*pt-EDD and pt-EDA indicate genes encoding EDD and EDA with an added plastid targeting signal at the N-terminus. The plastid targeting signal is from the small subunit of Rubisco from pea (Cashmore, 1983).

TABLE 17
Transformation cassettes for seed specific expression of
the pt-EDD, pt-EDA, pt-ZWF, and pt-PGL genes in soybean

| | | |
|---|---|---|
| Promoter | Gene | Terminator |

Expression Cassette 1: pt-EDD expression cassette

| | | |
|---|---|---|
| seed-specific promoter | pt-EDD gene from | Terminator from the |
| from the soya bean | Zymomonas mobilis | soya bean oleosin |
| oleosin isoform A gene | (SEQ ID NO: 160) | isoform A gene |
| (SEQ ID NO: 11) | | (SEQ ID NO: 164) |

Expression Cassette 2: pt-EDA expression cassette

| | | |
|---|---|---|
| seed-specific promoter | pt-EDA gene from | Terminator from the |
| from the soya bean | Zymomonas mobilis | soya bean oleosin |
| oleosin isoform A gene | (SEQ ID NO: 161) | isoform A gene |
| (SEQ ID NO: 11) | | (SEQ ID NO: 164) |

Expression Cassette 3: pt-ZWF expression cassette

| | | |
|---|---|---|
| seed-specific promoter | pt-ZWF gene from | Terminator from the |
| from the soya bean | Zymomonas mobilis | soya bean oleosin |
| oleosin isoform A gene | (SEQ ID NO: 162) | isoform A gene |
| (SEQ ID NO: 11) | | (SEQ ID NO: 164) |

Expression Cassette 4: pt-PGL expression cassette

| | | |
|---|---|---|
| seed-specific promoter | pt-PGL gene from | Terminator from the |
| from the soya bean | Zymomonas mobilis | soya bean oleosin |
| oleosin isoform A gene | (SEQ ID NO: 163) | isoform A gene |
| (SEQ ID NO: 11) | | (SEQ ID NO: 164) |

### Soybean Transformation

Transformation can occur via biolistic or Agrobacterium-mediated transformation procedures.

For biolistic transformation, the purified expression cassettes selected from TABLE 16 and 17 are co-bombarded with the expression cassette for the hygromycin resistance gene into embryogenic cultures of soybean Glycine max cultivars X5 and Westag 97 to obtain transgenic plants.

The transformation, selection, and plant regeneration protocol is adapted from Simmonds (2003) (Simmonds, 2003, Genetic Transformation of Soybean with Biolistics. In: Jackson J F, Linskens H F (eds) Genetic Transformation of Plants. Springer Verlag, Berlin, pp 159-174) and is performed as follows.

Induction and Maintenance of Proliferative Embryogenic Cultures: Immature pods, containing 3-5 mm long embryos, are harvested from host plants grown at 28/24° C. (day/night), 15-h photoperiod at a light intensity of 300-400 μmol m⁻² s⁻¹. Pods are sterilized for 30 s in 70% ethanol followed by 15 min in 1% sodium hypochlorite [with 1-2 drops of Tween 20 (Sigma, Oakville, ON, Canada)] and three rinses in sterile water. The embryonic axis is excised and explants are cultured with the abaxial surface in contact with the induction medium [MS salts, B5 vitamins (Gamborg O L, Miller R A, Ojima K. Exp Cell Res 50:151-158), 3% sucrose, 0.5 mg/L BA, pH 5.8), 1.25-3.5% glucose (concentration varies with genotype), 20 mg/l 2,4-D, pH 5.7]. The explants, maintained at 20° C. at a 20-h photoperiod under cool white fluorescent lights at 35-75 μmol m⁻² s⁻¹, are sub-cultured four times at 2-week intervals. Embryogenic clusters, observed after 3-8 weeks of culture depending on the genotype, are transferred to 125-ml Erlenmeyer flasks containing 30 ml of embryo proliferation medium containing 5 mM asparagine, 1-2.4% sucrose (concentration is genotype dependent), 10 mg/l 2,4-D, pH 5.0 and cultured as above at 35-60 μmol m⁻² s⁻¹ of light on a rotary shaker at 125 rpm. Embryogenic tissue (30-60 mg) is selected, using an inverted microscope, for subculture every 4-5 weeks.

Transformation: Cultures are bombarded 3 days after subculture. The embryogenic clusters are blotted on sterile Whatman filter paper to remove the liquid medium, placed inside a 10×30-mm Petri dish on a 2×2 cm² tissue holder (PeCap, 1 005 μm pore size, Band SH Thompson and Co. Ltd. Scarborough, ON, Canada) and covered with a second tissue holder that is then gently pressed down to hold the clusters in place. Immediately before the first bombardment, the tissue is air dried in the laminar air flow hood with the Petri dish cover off for no longer than 5 min. The tissue is turned over, dried as before, bombarded on the second side and returned to the culture flask. The bombardment conditions used for the Biolistic PDS-I000/He Particle Delivery System are as follows: 737 mm Hg chamber vacuum pressure, 13 mm distance between rupture disc (Bio-Rad Laboratories Ltd., Mississauga, ON, Canada) and macrocarrier. The first bombardment uses 900 psi rupture discs and a microcarrier flight distance of 8.2 cm, and the second bombardment uses 1100 psi rupture discs and 11.4 cm microcarrier flight distance. DNA precipitation onto 1.0 μm diameter gold particles is carried out as follows: 2.5 μl of 100 ng/μl of DNA encoding each transgene expression cassette of interest (TABLE 16 and 17) and 2.5 μl of 100 ng/μl selectable marker DNA (cassette for hygromycin selection, TABLE 16 and 17) are added to 3 mg gold particles suspended in 50 μl sterile dH₂0 and vortexed for 10 sec; 50 μl of 2.5 M CaCl₂ is added, vortexed for 5 sec, followed by the addition of 20 μl of 0.1 M spermidine which is also vortexed for 5 sec. The gold is then allowed to settle to the bottom of the microfuge tube (5-10 min) and the supernatant fluid is removed. The gold/DNA is resuspended in 200 μl of 100% ethanol, allowed to settle and the supernatant fluid is removed. The ethanol wash is repeated and the supernatant fluid is removed. The sediment is resuspended in 120 μl of 100% ethanol and aliquots of 8 μl are added to each macrocarrier. The gold is resuspended before each aliquot is removed. The macrocarriers are placed under vacuum to ensure complete evaporation of ethanol (about 5 min).

Selection: The bombarded tissue is cultured on embryo proliferation medium described above for 12 days prior to subculture to selection medium (embryo proliferation medium contains 55 mg/l hygromycin added to autoclaved media). The tissue is sub-cultured 5 days later and weekly for the following 9 weeks. Green colonies (putative transgenic events) are transferred to a well containing 1 ml of selection media in a 24-well multi-well plate that is maintained on a flask shaker as above. The media in multi-well dishes is replaced with fresh media every 2 weeks until the colonies are approx. 2-4 mm in diameter with proliferative embryos, at which time they are transferred to 125 ml Erlenmeyer flasks containing 30 ml of selection medium. A portion of the proembryos from transgenic events is harvested to examine gene expression by RT-PCR.

Plant regeneration: Maturation of embryos is carried out, without selection, at conditions described for embryo induction. Embryogenic clusters are cultured on Petri dishes containing maturation medium (MS salts, B5 vitamins, 6% maltose, 0.2% gelrite gellan gum (Sigma), 750 mg/l MgCl₂, pH 5.7) with 0.5% activated charcoal for 5-7 days and without activated charcoal for the following 3 weeks. Embryos (10-15 per event) with apical meristems are selected under a dissection microscope and cultured on a similar medium containing 0.6% phytagar (Gibco, Burlington, ON, Canada) as the solidifying agent, without the additional MgCl₂, for another 2-3 weeks or until the embryos become pale yellow in color. A portion of the embryos from transgenic events after varying times on gelrite are harvested to examine gene expression by RT-PCR.

Mature embryos are desiccated by transferring embryos from each event to empty Petri dish bottoms that are placed inside Magenta boxes (Sigma) containing several layers of sterile Whatman filter paper flooded with sterile water, for 100% relative humidity. The Magenta boxes are covered and maintained in darkness at 20° C. for 5-7 days. The embryos are germinated on solid B5 medium containing 2% sucrose, 0.2% gelrite and 0.075% MgCl₂ in Petri plates, in a chamber at 20° C., 20-h photoperiod under cool white fluorescent lights at 35-75 μmol m⁻² s⁻¹. Germinated embryos with unifoliate or trifoliate leaves are planted in artificial soil (Sunshine Mix No. 3, SunGro Horticulture Inc., Bellevue, Wash., USA), and covered with a transparent plastic lid to maintain high humidity. The flats are placed in a controlled growth cabinet at 26/24° C. (day/night), 18 h photoperiod at a light intensity of 150 μmol m⁻¹ s⁻¹. At the 2-3 trifoliate stage (2-3 weeks), the plantlets with strong roots are transplanted to pots containing a 3:1:1:1 mix of ASB Original Grower Mix (a peat-based mix from Greenworld, ON, Canada):soil:sand:perlite and grown at 18-h photoperiod at a light intensity of 300-400 μmolm⁻² s⁻¹.

T1 seeds are harvested and planted in soil and grown in a controlled growth cabinet at 26/24° C. (day/night), 18 h photoperiod at a light intensity of 300-400 μmol m⁻² s⁻¹. Plants are grown to maturity and T2 seed is harvested. Seed yield per plant and oil content of the seeds is measured.

The selectable marker can be removed by segregation if desired by identifying co-transformed plants that have not integrated the selectable marker expression cassette and the desired transgene expression cassettes into the same locus. In this case, plants are grown, allowed to set seed and germinated. Leaf tissue is harvested from soil grown plants and screened for the presence of the selectable marker cassette. Plants containing only the desired transgene expression cassettes are advanced.

### Example 9. Use of Genome Editing to Insert Pt-EDD, Pt-EDA, Pt-ZWF, and Pt-PGL into the Genome of Plants

There are multiple methods to achieve double stranded breaks in genomic DNA, including the use of zinc finger nucleases (ZFN), transcription activator-like effector nucleases (TALENs), engineered meganucleases, and the CRISPR/Cas system (CRISPR is an acronym for clustered, regularly interspaced, short, palindromic repeats and Cas an abbreviation for CRISPR-associated protein) (for review see Khandagal & Nadal, Plant Biotechnol Rep, 2016, 10, 327). CRISPR/Cas mediated genome editing is the easiest of the group to implement since all that is needed is the Cas9 enzyme and a single guide RNA (sgRNA) with homology to the modification target to direct the Cas9 enzyme to desired cut site for cleavage. The sgRNA is a synthetic RNA chimera created by fusing crRNA with tracrRNA. The guide sequence, located at the 5′ end of the sgRNA, confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Cas9 expression plasmids for use in the methods of the invention can be constructed as described in the art. The ZFN, TALENs, and engineered meganucleases methods require more complex design and protein engineering to bind the DNA sequence to enable editing. For this reason, the CRISPR/Cas mediated system has become the method of choice for genome editing.

The CRISPR/Cas technology, or other methods for genome editing, can be used to insert expression cassettes for pt-EDD, pt-EDA, pt-ZWF, and pt-PGL into the genome of plants at a defined site using the plants homologous directed repair mechanism. A sgRNA with a guide sequence for the genomic location of interest is used to enable the Cas enzyme, or other CRISPR nuclease, to produce a double stranded break in the genome. An expression cassette containing a seed specific promoter, the Entner-Doudoroff pathway gene of interest, and an appropriate 3′ UTR sequence is flanked by sequences with homology to the upstream and downstream region of the sgRNA cut site. This expression cassette is inserted into the double stranded break in genomic DNA using the homologous directed repair mechanism of the plant.

There are many variations of the CRISPR/Cas system that can be used for this technology including the use of wild-type Cas9 from Streptococcus pyogenes (Type II Cas) (Barakate & Stephens, 2016, Frontiers in Plant Science, 7, 765; Bortesi & Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187), the use of a Tru-gRNA/Cas9 in which off-target mutations were significantly decreased (Fu et al., 2014, Nature Biotechnology, 32, 279; Osakabe et al., 2016, Scientific Reports, 6, 26685; Smith et al., 2016, Genome Biology, 17, 1; Zhang et al., 2016, Scientific Reports, 6, 28566), a high specificity Cas9 (mutated S. pyogenes Cas9) with little to no off target activity (Kleinstiver et al., 2016, Nature 529, 490; Slaymaker et al., 2016, Science, 351, 84), the Type I and Type III Cas Systems in which multiple Cas protein need to be expressed to achieve editing (Li et al., 2016, Nucleic Acids Research, 44:e34; Luo et al., 2015, Nucleic Acids Research, 43, 674), the Type V Cas system using the Cpf1 enzyme (Kim et al., 2016, Nature Biotechnology, 34, 863; Toth et al., 2016, Biology Direct, 11, 46; Zetsche et al., 2015, Cell, 163, 759), DNA-guided editing using the NgAgo Agronaute enzyme from Natronobacterium gregoryi that employs guide DNA (Xu et al., 2016, Genome Biology, 17, 186), and the use of a two vector system in which Cas9 and gRNA expression cassettes are carried on separate vectors (Cong et al., 2013, Science, 339, 819).

It will be apparent to those skilled in the art that any of the CRISPR enzymes can be used for generating the double stranded breaks necessary for promoter excision in this example. There is ongoing work to discover new variants of CRISPR enzymes which, when discovered, can also be used to generate the double stranded breaks around the native promoters of the mitochondrial transporter proteins.

It will be apparent to those skilled in the art that any of the site directed nuclease cleavage systems can be used to generate the double stranded break in genomic DNA can be used insert expression cassette for pt-EDD, pt-EDA, pt-ZWF, and/or pt-PGL in this example.

### Example 10. Transformation of Plants with Expression Cassettes for EDD, EDA, GDH, and GCK

It may not be necessary to express genes encoding GDH and GCK with genes encoding EDD and EDA, as 6-phosphogluconate is already produced by the plastidic ZWF protein of the OPP pathway in many plants, and is dominant in camelina seeds (Carey et al., 2020, Plant Physiol. 182:493-506). However, additional expression of GDH and/or GCK could encourage glucose to be catabolized by the ED pathway instead of the EMP pathway (FIG. 1). There are many sources of GDH and GCK enzymes that can be used. As an illustrative example, DNA encoding the signal peptide of the small subunit of Rubisco from pea and the first 24 amino acids of the mature protein (Cashmore, 1983) can be fused to the gene encoding GDH from E. coli (SEQ ID NO:32) and the gene encoding GCK from E. coli (SEQ ID NO: 33). The plastid-targeted GDH and GCK genes can be expressed from seed specific promoters in a variety of plants. Co-expression of the plastid targeted GDH and GCK cassettes with plastid targeted EDD and EDA expression cassettes may provide additional carbon conversion efficiency.

### Example 11. Insertion of EDD, EDA, ZWF, and/or PGL into the Plastome Through Direct Plastid Transformation of Plants

Alternatively, genes encoding the EDD and EDA enzymes can be inserted into and expressed directly from the plastid genome. In this scenario, a plastid targeting signal is not needed since the protein is produced in the plastid. Genetic constructs used for plastid-encoded transgene expression in a host organism typically comprise in the 5′-3′ direction, a left flank which mediates—together with the right flank—integration of the genetic construct into the target plastome; a promoter sequence; a sequence encoding a 5′ untranslated region (5′ UTR) containing a ribosome binding site; a sequence encoding a gene of interest, such as the genes disclosed herein; a 3′ untranslated region (3′ UTR); and a right flank. Plastid gene expression is regulated to a large extent at the post-transcriptional level and 5′ and 3′ UTRs have been shown to impact RNA stability and translation efficiency (Eibl et al., Plant J 19, 333-345 (1999)). Due to the prokaryotic nature of plastid expression systems, one or more transgenes may be arranged in an operon such that multiple genes are expressed from the same promoter. The promoter driving transcription of the operon may be located within the genetic construct, or alternatively, an endogenous promoter in the host plastome upstream of the transgene insertion site may drive transcription. In addition, the 3′UTR may be part of the right flank. The construct may also comprise selectable marker gene(s) and other regulatory elements for expression.

Plastid transformation constructs can be made with combinations of genes selected from EDD, EDA, GDH, GCK, ZWF, and/or PGL. Insert sites for the constructs can be varied by altering the sequence of the left and right flanking regions, which direct the expression cassettes to the desired region of the plastome. These constructs can be transformed into a variety of plants. For example transformation of the cassette into Camelina and selection of the transformant can be performed. The plastid insert can be confirmed by PCR of plastid genomic DNA or alternatively can be confirmed through Southern blotting procedures. The percent homoplasmy (the amount of plastid DNA containing the insert at the desired site) can be estimated through Southern blotting procedures. It is desirable to isolate homoplasmic plants (plants that have all of the plastid DNA containing an insert at the desired site). Homoplasmic plants are grown in a greenhouse. T1 seeds are isolated. T1 plants are grown to produce T2 seeds. T2 seed yield and oil content is determined.

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “YTEN-62241WO-Sequence-Listing_ST25.txt”, created Feb. 16, 2021, file size of 622,592 bytes, is hereby incorporated by reference.

1. A genetically engineered plant that expresses a 6-phosphogluconate dehydratase (EDD) and/or a 2-keto-3-deoxy-6-phosphogluconate aldolase (EDA), the genetically engineered plant comprising at least one of a first modified gene or a second modified gene, wherein:
the first modified gene comprises (i) a first promoter and (ii) a nucleic acid sequence encoding the EDD;
the first promoter is non-cognate with respect to the nucleic acid sequence encoding the EDD;
the first modified gene is configured such that transcription of the nucleic acid sequence encoding the EDD is initiated from the first promoter and results in expression of the EDD;
the second modified gene comprises (i) a second promoter and (ii) a nucleic acid sequence encoding the EDA;
the second promoter is non-cognate with respect to the nucleic acid sequence encoding the EDA; and
the second modified gene is configured such that transcription of the nucleic acid sequence encoding the EDA is initiated from the second promoter and results in expression of the EDA.

2. The genetically engineered plant according to claim 1, wherein the EDD is characterized as EC 4.2.1.12.

3. The genetically engineered plant according to claim 1, wherein the EDD converts 6-phosphogluconate (6PG) to 2-keto-3-deoxy-6-phosphogluconate (KDPG) and water.

4. The genetically engineered plant according to claim 1, wherein the EDD is one or more of a bacterial EDD, a cyanobacterial EDD, an algal EDD, or a plant EDD.

5. The genetically engineered plant according to claim 1, wherein the EDD has at least 30% or higher sequence identity to one or more of the following:
(1) Zymomonas mobilis EDD of SEQ ID NO: 44;
(2) Synechocystis sp. PCC 6803 EDD of SEQ ID NO: 136;
(3) Guillardia theta EDD of SEQ ID NO: 140; or
(4) Hordeum vulgare EDD of SEQ ID NO: 139.

6. The genetically engineered plant according to claim 1, wherein the EDD comprises one or more of the following:
(1) Zymomonas mobilis EDD of SEQ ID NO: 44;
(2) Synechocystis sp. PCC 6803 EDD of SEQ ID NO: 136;
(3) Guillardia theta EDD of SEQ ID NO: 140; or
(4) Hordeum vulgare EDD of SEQ ID NO: 139.

7. The genetically engineered plant according to claim 1, wherein the EDA is characterized as EC 4.1.2.14.

8. The genetically engineered plant according to claim 1, wherein the EDA converts 2-keto-3-deoxy-6-phosphogluconate (KDPG) to pyruvate and D-glyceraldehyde 3-phosphate.

9. The genetically engineered plant according to claim 1, wherein the EDA is one or more of a bacterial EDA, a cyanobacterial EDA, an algal EDA, or a plant EDA.

10. The genetically engineered plant according to claim 1, wherein the EDA has at least 30% or higher sequence identity to one or more of the following:
(1) Zymomonas mobilis EDA of SEQ ID NO: 70;
(2) Synechocystis sp. PCC 6803 EDA of SEQ ID NO: 137;
(3) Phaeodactylum tricornutum EDA of SEQ ID NO: 92; or
(4) Hordeum vulgare EDA of SEQ ID NO: 97.

11. The genetically engineered plant according to claim 1, wherein the EDA comprises one or more of the following:
(1) Zymomonas mobilis EDA of SEQ ID NO: 70;
(2) Synechocystis sp. PCC 6803 EDA of SEQ ID NO: 137;
(3) Phaeodactylum tricornutum EDA of SEQ ID NO: 92; or
(4) Hordeum vulgare EDA of SEQ ID NO: 97.

12. The genetically engineered plant according to claim 1, wherein the genetically engineered plant expresses both the EDD and the EDA.

13. The genetically engineered plant according to claim 12, wherein the genetically engineered plant comprises both the first modified gene and the second modified gene.

14. The genetically engineered plant according to claim 12, wherein the genetically engineered plant comprises the first modified gene, lacks the second modified gene, and further comprises an endogenous gene encoding the EDA.

15. The genetically engineered plant according to claim 12, wherein the genetically engineered plant lacks the first modified gene, comprises the second modified gene, and further comprises an endogenous gene encoding the EDD.

16-18. (canceled)

19. The genetically engineered plant according to claim 1, wherein the genetically engineered plant exhibits increased expression of the EDD and the EDA relative to a reference plant that does not comprise the at least one of the first modified gene or the second modified gene.

20. The genetically engineered plant according to claim 1, wherein the genetically engineered plant exhibits increased expression of the EDD and the EDA in plastids of cells of the genetically engineered plant relative to a reference plant that does not comprise the at least one of the first modified gene or the second modified gene.

21-23. (canceled)

24. The genetically engineered plant according to claim 1, wherein the genetically engineered plant has an increased sink strength in comparison to a reference plant that does not comprise the at least one of the first modified gene or the second modified gene.

25-26. (canceled)

27. The genetically engineered plant according to claim 1, wherein the genetically engineered plant comprises one or more of Camelina sativa, camelina species, Brassica species, Brassica napus (canola), Brassica rapa, Brassica juncea, Brassica carinata, crambe, soybean, sunflower, safflower, oil palm, flax, or cotton.

28. (canceled)

29. A method for producing the genetically engineered plant of claim 1, the method comprising a step of:
(1) introducing at least one of the first modified gene or the second modified gene into a plant, thereby obtaining the genetically engineered plant.

30-32. (canceled)


SEQUENCE LISTING (164 sequences)

SEQ ID NO: 1 - DNA, 770 bp, Cauliflower mosaic virus
agagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcg

SEQ ID NO: 2 - DNA, 1148 bp, Glycine max
gcaacagaagacccaaaactcaaaaaagttagtttcgggccaacatttcctcttgagggatgacacgtgacctgctactctggcccttatctggcatgtccatccttcttggcgcgacatttaattcgtcgtcagaaataactgaaggacaccttgcttgtttctcttttggccgccaccggtcttgtcatcgtcgaaggcgcccttgcgcttgtcggcagaacctttttcggcgacctccttgccttttcctttggccttgttcgtcatttctacagagaatgcaatgagaccaacgccaattgcatggttagagttagagaaatggagagaggaagaagtgcgtgactagagtgtgtgtaactgtgaagaacgacgagtccaaaatgaattttactgtaaataatttgaggaaaaaagtgatcaatacatatcatgcggtgcatacaagaatcggccattggtcaacttgtgagaggaaaaaatcatttaactaataccaaataatcttaaaattaataaaataatttaactaattaacccacggaagaaccttcttccgttgactctggcggaagaagttcttccgcatagttccatggaagatggttcttccgcagttcttctttcgttgacactcgcggaagaaatgttccacgggcgtccgcggaagaactttcttccgcaaagctaaagagcatttttgccatgtcgaaatcatcgccaatgaccagggtaacagaaccacgccctcttatgttggtttcaccgattcagagcgtttgatcggtgatgccgccaagaatcaggtcgccatgaaccccgtcaacaccgtcttcggtaagatccctagccgacacttcgccttttcaggatttgcattgttcctagatttttggatctgttgtttgaaactccacttttctattttggtaatttttagttttattttgtaatcctgctgtttatatgtcttattgttattattaatcgttgcatggtctgaactggtttagaactctacttgtattgtttgttaaaatcttatttgaaatcgaatagtaatataattttaatcgaatggtgatatgcataaacatcgtatttgttcgtcgaattctggttttgaattgaataatattgttatg

SEQ ID NO: 3 - DNA, 1378 bp, Glycine max
ctagaaattaaatgtttttaacaggtaatttgagaaaaatgtacttcaaaataattagttttaccagtttatgtcttctttttctcttttttatctttattctatgtttcaaattctaataatacatcatttaaatatttttaatttaaaagtgcttactaaattttaaaaaaatcatatttatcaaataacttctactttaaatttaaacttcattatttttaacttaaaaataacttttaaattaaaaaaatgaaaacaaacactacctaaaccctaaacactatctatctaagtcacattacttaatgattcttaatttatgttctttgtaaactttcatttcttcctccttttggctatacatgttcatttctgtgtactttactatattattagtaaaagccttttatataggtatatcaaatcaaataattaatataatatataattctcttaatttcatttcttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaag

SEQ ID NO: 4 - DNA, 1500 bp, Glycine max
gatcctcacaaacctcacttggagacataggtgtgagggtaacctttttccctttatgtacaaatgaaaatttgtttgtgacaccattatggacaacatccttacactactaaaaaagcttttttttacgacatcatatttacgacagtcatacaaaaacgtcttagtatgtataaggatggcaatttcgtaaatatttcaaacatttcaaaggcagtttcagaaaaccgtctttgaatgcggccattttaatttttaacgcgcccctcgcatccgttcctcttctttccgcaaatgtggtgctcgttccttttctttcccagctggcatctgttcctctccccactcgctagctatcttctgcttctcctcttctctcctcttcccattacatttctccaccttctccctggtaccaccaccgccccccactccacattcgtcctccgcccccattcccctatcctccagtaaaattacaaaaaaccctaacaccaaaaaaacccaaacccctgtcgcaatgaaatctccacccccaaatagctctttggaatagaatcaaggaacttaccaaatccattatatgctattggggttttggcatgtttccggtgtgaaagaaggaaaaagaaatgcgtatgcgatggtgatgtacgtaggtacgccgaaggactacgaattctacatagccatactcgtgcttctcaaatcgctggctacgctcgacgttgaaattgatcttgctgtgattgcttcccttgatgttcctcctcgatggattcgagctctgtaagtctcactccttcaccatcatttgccactttatttttatgtacttttactttattattatttgtaacctgtatttttatttggtttcggatatctgttgctttattattcaccctggaatttggttgattttattatttttgaaaaataaggaaagagatttatttgttagcttaattgttttaattggcgaatatgtttttcttttcccttttttgcacagagtgaagctttgttcttagggtaatggattcccttttttgtgatgctagtggatgatttgactgattagtgtttagtggaatgaagaaccagaactagtagtaggtagagggaatcacttttggttttggatgtaaacttagaaatgtgcagcactgcacagaattgatatttgatcgtgggtcaaattgtcaaaatgtgcaaagaatacaaaggcacaggtgatatcattccattttacgttttttaacgaagctgttagtttcaattcaattatttacatatataataaatatattgatacttgctttagtttcatgaattaaaagaatttgattttgtaaatttcatttgaatttgtttttgtacaagctctcaacttttattatatgaacgagaagtttcttttttcctttttgagtttatttgaacttgtggtgttctaattgtatatatttttgtgcaggtgtcaatcggtactactac

SEQ ID NO: 5 - DNA, 1261 bp, Glycine max
atctctcgacagttgcgaactgaacgctgagttggtaatgctatgccctatcgctttttgcaccgtcccatgatcatttcccccacaccaccccatcaacctctaaaaagttaagagtgaaaattacacacacccgaggagaagaaaagctgcttcttctaagcatcacaacctagttactttacttgtagggccttttccatttcccctaaattacccctcttttcatcatatgataataatatccagctcagactatagtatgatattatgatgtcagcataataggttggcactaaagtcttaaagggcattgtacatgttgcacctggcattcaaattcataaatactaacactgtgaaatagattataaatcctcaaataaatgtcacacggttggggttcgaatccactcaaaaaggctaatgggatgggatttaagtgccaaggaatataccatggactttaacagcaacacaatttacaatctaaaatgtattacttttttttttcaaaaaagatatacaaaataaggtaccaagaataaaaggagtatttagaaacagtggcaccaatttaataaattatttatataaaatgacacttatttaatttatcaatgataaaagtaatattgatttattctctgattaactgttcaattaatagtgttattatcataatctgtcgcaaaagttatttttatcaacaacaataattgatacaagtagtataaaattaagcctcttagttaatatagactacttgatactaaaaccatgttacaccaaaaagtaatttttatgtcacttgtctatataataattacgactaaattaataatttttaaaaatattactgaatccattaaccgaacttttataatgaaagtatttttatgctttaaaatcacaaacattgaataaactaaaaatgataccacggaattggaacaagagacgttccacacaaaagaaaaaaatatgttgaataattgaaacggtgacaagaaaagtggaataataatacaaagatggcagatggggttattgttattggaggagatgagtgaaataatgagtgaggggggtgtaactggaaagcaagaaaaagcgcaagagtgccagctatttccaacaacaaacgtggcccgtgggatgcgatattcgtaacgaacggcgaggatggaaggacgtgcaatttgcgcttcatttgaggcgaatttcatttggccagaccttccttttttaaaccacaggg

SEQ ID NO: 6 - DNA, 1094 bp, Glycine max
tgtgtcaatgttgtttctggtgaattgacataatgaattctacctgtacggagtagagaataactatttacccaacaagaatgattatctcattaatttttgaagtagacgcaataacgaatatattatacattcagaaaaatttcaccatattattctcaaatcacaacaataatttgttttttttttgcttgatataaaaccaatactctatactttttaaggttaatttaaacttaaagagtatttttaagatgcatgtactttaaggaataatagaaacatgacaacatcataaaagaatgaagaaactgaatcataacgtagtttgttacgccttccatttggtggttgatttggatacaatctagattggtttgctaaatggtttataagttatgtagacgtttttattactactattttagacaaatcaaatacacaccttcactttattctattcaaataacatgatttttcctaacattttttaaaaaaattactttttaaatataaactaattattttagaaatagttttataaaaatccacgccaaaaaaattaagttgtttttataaatataaacatcgggcttcaatcttaaatttataaatgtacgaaataatttgacagttaaatggaaattgctagcatggaagtgtttttatcatttatcaaactcaaccaaactgaacatcagaataattattagtgacaaattttgcagcatatgaagtggcttgcatagctccaaggctggcgatcatatgtcagattagagcaggctctctttggtactatgatacatttcaagcaaataacaaccgtaaaaattcacgccaaaatttttggaacgaatctatatattattattttatttcttttgatttcatgtacgtacagtgcccgtaattgacatgtctttgttccttaatgcctttcccacgtggaacaggcacctagaaacttggactaagtagggaattgagggccatggactatagtgccaaaccaacatcattttatatatatatatatatatatatatatatatgctattgttttctatagtttttggaaattaatacttatc

SEQ ID NO: 7 - DNA, 1449 bp, Glycine max
atttgtactaaaaaaaaatatgtagattaaattaaactccaattttaattggagaacaatacaaacaacacttaaaacctgtaattaatttttcttctttttaaaagtggttcaacaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttc

SEQ ID NO: 8 - DNA, 1321 bp, Glycine max
aaaaacacaaaaaaaaattatacaaaaatgtttctcacaacatgagaagtaaaatccctcaaagaatttcacatcatcatatcagaatcaaaggaatcaaaatcataggtcaaaaatacaaaaacaccaagaacactcaatttattaactaatttgcatcatgacatcaattggtccatcaaacacaacaatcttgtaattataatcgtaacgaaagaattacaatgcaataaacatcccaaaataaacctcaatttaatcctctaaggatccctatacatgttcattctaaccccaattgtgataaattcatcccttacctctaagcaggctcacgtgtgtagtctggcagtgatagaggcatctctagtggttttctaatagtcctcaagcttgtttttcctctagttgttctgttaggattttcaagcgttagagagaagaagaagagattggagcctctatttcactgttaccgtacaagggatatttttctcaccataaacattattttgcaaatcccaacgaaggagatgtccgtacataagttcgaaacctggtgctcgaatttcacgacgattcaatggttaacaagtccaagattgtatttttactgtgacagatttgagtgtatacaagaaaaagagagctccatgcgaggaatatttctctcacagtagacattatttcataaatcccaatggtaaaaatatgcaaaaatgagtttcaaacctgcttttaaaatttcatgacgactcaacggttaacgtgtccgggattatattttcactggaacaagtttgagtgcatgcgggaaaagagagggttttgggagaggaaaaaaggaaaacaaatttaagaggaagagagagcgtaaaaatttatcgtaaatgtaaaaaatgacctaatatatctctatttataactagggtactctcaatctattatttactcatttttttattttattattttataaaaaagaattttattttacttcctatcaaattaataaataaaacattcttcttattttctaagatcacatatttattttatttaccttaaaatcatcattttaattaataaaattatttcttcttatttatttaattacaaaaatcttattatttttttaaaattttatttatttttaaataaaatattttttaatttattttataaaaaatgagatgttacattgaattataaaataaatagccaacaataaatagccgacttgcttttgcattgactaaggaagtcaagtcatcaataaatataatttccagttggcaatattctcaaagttggtctatat

SEQ ID NO: 9 - DNA, 514 bp, Glycine max
agatttgatcgatacttcattaaattgacattttattttaacacataatacattattaaaaatataaataaacatttacagcgaagttatataattaaaagcctggtctatgtaatggtaggaaatttgaaaatctaaaagcaaacaaaaattgttgtttatggtgctaagttgcacctggaaagatgcattgtttagctaaaacattcacgtcgagtacttggtttgggaaaaaaagccattcaagcttagctggtcctctctcctgtctctctctctctgtctgtctctctctgtctgtctctctctcaagcacatacacaaacaaagtaagggctataaataggagggatggaagtggaagaaagtctatagcgaagtttcatttctttggattagaaatttttcccaaagctgatcgagaagccagccaggccaggtctgtagttttctttttttctttttaatattaattcattattgtgttcttcatcatataatataattaagcctt

SEQ ID NO: 10 - DNA, 702 bp, Glycine max
cgcgccgtacgtaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgc

SEQ ID NO: 11 - DNA, 579 bp, Glycine max
acgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgc

SEQ ID NO: 12 - DNA, 1500 bp, Oryza sativa
gcagctgttttcgcggtacagggtgcaacaaaagcccatgacggcccacacctgcctctctccgctccaaacaccgaaacaagggggtgggtgcaatgggccggcgctcgaagaccgcgaactctttccaacagcccagcgcattagcccctcctcctactctctctaccttctttttaacatgcgactttctttctgtggacgacggcatcaacgacgggagcaggagcgggggctgaagcacggtgcgtgggctcctggagtggcgacggcctctccggcgagcttcctctggcgaactccctccgctcctcctatggcgaaatccaaacaagggtcagtttcgactccaaccttctcccaccaccacctcctgaccgtgccaccacccggccttgtcggcactgaaaggcgtcaacttgtcagcgcgggcctgctcggtcggtctcctcctcccctatttcgtttagctttgcccccgccaccaacaccggcccacggcccatggccgaccccgcggctttggcgccgccatcgctatctcgccgctgtccttttttcatgaccttcggtgccatccctctaaattcgatgcacctccctggctctatctccctttacctccgaaatcctaaccctacccataatctctagtgagtcttgtctttatttatggcctctttgaatcgcaggattgataaaacgtaggattttgataggaatgtaagtgtaaaacacatgattgtaaaatagaggaaaaacataggaatggccgtttgattgaaccgcagaaaaaacacaggaattagatgagagagatagactcaaagttactaagagattgaagcttttgctaaatttcctccaaaatctctataggattggccattccatagaaatttcaaaagatttaataggattcaatcctttgtttcaaaaaacttcatagaaaatttttctatagaattaaaatcctctaaaattcctatgttttttctccaattcaaaggggcccttaggttggaatttggaaagtgttcgcgagaaatcaagcggtcgcacgttagcgaattaggatttccggaaacaaaggaccgactccgcctatccatcgtcacgagcacagtgtagaacctcccagacctcaagagaccgttcaaaaagcgcgcgcccaagcggggcccaccaacgcgtccccaccgtgtcgcctcctgattggttgtcccctcttcctttcacgcgaaccggcaccctcccgacccttccagaacccccaatccgacggccaggatcgcccgcgcgcgaacgttctagacccccgccacctccgccacaaaacctctgcccctcccctctccccccgcttcgtctcgttcgagaaatcagaaagagagagaaattcccacgcagcagcaagcaatccaatccgagagcgcgcgtttgcgattattcgctttcgattccgcgaggtttttggagagggaggagaaggaggaggag

SEQ ID NO: 13 - DNA, 1500 bp, Oryza sativa
acagcatttattgtagtctggtcaagcgtgtcacgctgcatgcaacgcagtacagcgcgttcctttacccggtctgtgaccagtcacagaccggtcagatcacgggttaggtggcgactggcggtctgacgcacgccttgccccatcccgtcaagacgaaagcctctaggcactcgtctcaagccggagctagcgtgttatctcttagagatggcacgttagccctggttagatttataccaggcttcatcctaaccattacaggcaaggtgttacacgaagaagggcaaaacatgcacgttgttaaactgacgcgtgggggacaagaatgaccggtctgacactggtcgcatcagcaacgggcagccacgatcccgcgtcatctccgtctccgccgggagtggaggtaggtgtgggctgtcccatcagaagggctcccggatggaaaccgtaccgatctccgcccattaaagagaaaaagaacagtccagtttggaaagagaagggtgcatgtggtatccccttgaagtataaaaggaggaccttgcccatagagaagggggttgattctttccagattcagagcctagaacgagggagaggtgggctcacactttgtaacttgtccatacacaaatccacaaaaacacaggagtagggtattacgcttccgagcggcccgaacctgtatagatcgtccgtgtctcgcgtttcttgctggctgacgatccttccacatacagagagagagagagcttgggatctcaccctaagcccccggccgaaccggcaaaggggggcctgcgcggtctcccggtgaggagcctcgagctccgtcagacatgttcagtttcattatattatgaaatgtcacgtactgtttgttctagttagtgaattgtcatatggtaagaatatataaaaattaggttttctggactctatcttccaatgtatttttggatcctataacaaaatattttcataaatatattttttaagaatctaaacttttttgaaataaaagagcaacaaagaaaataaaaacgctctctcgtaagtaactcgtgaagatccatcgagagccactcgtttgaatcgtcgacacaaaagaacacttcattgattgcttttcgtcaattagccgcacagcacagtactctccaatctgctaaaccaaaaccaatctcatccatccatacccttcttgacaccaagtggcaactcctgattggacgcgccctatcctacatggcacccccaagattctctcgataggctacaggggccacaccgaccctccacgtcatcgtccacgtcaccctcatcccggcccatccagccaatcccagcccagcaaaaaatcttcccaagtggccaccagataagcctctccacgtattaatacgccaagtgttcgtcgccatgacacagcacgcacacacaccccaccagcagcagcagcagtagctgagcttgaagcagcagagcgaggtagac

SEQ ID NO: 14 - DNA, 1500 bp, Oryza sativa
tccacctctgttggttgcatcgacgtcgcttccctagctcccgtctctagtccggatcctattcctccttggagaccgaagctaccgcaaccattgctcggtggttagcgagcgtggagctgtcctccccactttcgcgtcctcgttcgccaccacagccatacttcgcatggtgatgtcttctccttcactcaccgctaaactcagtgcaaccgtttctaccctagccccggccgccgctctcatagaggtgaaagttcatttacatgtaggtcccacatgttttatgttttttatttttcttttactgattagcatgccacgtaaatcaaaacaacaatccatagtgttttaagtatttttatttaatacgtgagatggagtacaaaaacgagagatgcaaagtgaacttgctaaaacacattttctggttgattacagtcgcttgttgagccattggatcggtcataggattcgtgctagcatacttaattacgcgtaactagttgtgctttataggttacaggtcgctaattagcggtctactggagaactttgctactatttttttcttcactgcatgcactcgatcaagtatgagtatttgtaccgaccagcgaaacacatatgtaattaaagtataaatatgtaattagtatatattagtagtatatttagacagtagttacaccctacatacacaccacttacatatataattagtatgtaattttgtaacttacatatgtaattttagtacttacatatgtaattttgagacttacattgtaaatacactaaaattacatatgtaatttagtaacctacaatgtaaatacatgccgactaacttttgatgaaaaatatggtgttataaatatagctactcccgaactttattccttctctgtgagatatcagtggaaacgctcggtggaatcgggggagtatttgggagcacgcgccgacgcgcgcgtcgtgcgtgccgtcgtctttgtcgcggtggagcggagcgcgcccacttgcgcgcctgggccggaggcgggcgcgccgggggttcgggaatcccctggagccacacgtaaaggcgcgggcgggagggagggaggggccagctaggataaggcacgcgcggccgctgcgattggggcgcttgtgaacaccggggcgccacgtggagaggacgttacactccagccgccaaatttccactcccacacccgcgctcccctcccctctcttttccgtgatcgcacctcgcccacgcgccccccgccacacacaatctctgcagctctccagcttcgttggaactcgcgaatctctctccgatcccaggtaaagcagcgaacgacgtcacgcacgacgctgctcggtggatttcgttccttgctggggaaaaccatgcagagacgaaggtgaatgatctgcttttgtgtacttgcgtttaccaggtgaagcgcgagcttggagttggaggggagatcgatcagggccagg

SEQ ID NO: 15 - DNA, 1500 bp, Oryza sativa
ataattaattaattaatcaatcacttttcgtgctgtaaaaaatctcacccgatttgctgaaacgaactgagccgggcgactgtgatattctttcacgatttctgtttgtggcagtgggacattgctgtttattcgaaacaattttcaagtaaaaaaaaatactcaatggtaaggttgctagtaatagtttaacagtttgtttgcagctcagcaaatttcgtttcctcacagatgacacataactgaaagcactcaatgtaatgttgtgcttagctgctaaagcatgtcacgtcttagaaaacaactactccaccatggagaatttttcctcctacttactcctcacatacttaccatctccatataagttcccttgtcgtatcatatgtcttattcttcttgagcacagttattacagcagattttgtagaatagttatcgcatcaaaattttcctatgtcacctttgatcatgtgttatgtgtgcctcttgagtcttagggttaatgtggttgtaatgtgtttaaaaaactatatgaaagctcgtgtgttgctacgggagagagatacctcgaatgaatgtgagagatctccatttgagttgtgtaccttgagagagtgaaagatcacactatttatagacggttaataatggttactgaggtcgattcaccacatcgtcttaaacatttaatgagcatcctccacgtgaaaagtagagatgatagcgtgtaagagtggttcggccgatatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcataag

SEQ ID NO: 16 - DNA, 1945 bp, Oryza sativa
aaggtttcatgcgtatcgtgacagatgttacataatgacaaattccccagctggagcacctttatccctgctgtttgcatgaaattagcttgtcttgtagttccctccagcaaaaagaagtctgaaacaaaacaacatttcgaaaaaaaggcatccatgagttagcatttctacagttgtctatagaggggaaggctgcacgacaaagtttccaggcttggaaacaacctcttatgtaaaatttttcgtatgtatcagatgatttgtttgcgttacggcatctccacctaacatcaccttcatcatgcgcctatggtctttctcttgcctgttttatacgtaaaattggaaacgacagaaacttttgccatctttattaaaggaaggcaaatatgcaaatataggcatcaagatcacagttagtggattatcatctttgtaggttaacatgtcctaccccaggggagcttatactcaagtactccatgcattttcatgaaatgagaaaaaacgatttttaagagaaatgtactttcttgtatttatgccaaatggcaaggactgaaagggaaaaactaagaaagggaacgttacagtaaggctctgtggggactggggacttcagagaaacgtgaaccctgcttccttcctctgcatgaacataacaccagaggtttccagcctttcacacagttgttgatggcttcacacaattcatctctacctcctgactctttataaggacccccagcatcaccacaattgcacaagtacaggcattagatccacaagaacacttgggcaggcaagcacctctttgatctttaagccgttgttatgttctatttctgagcatatggtttctagttatattctttttcttcattcgtttcatatctttgaagtgttgatgcaaatgcggtgaacaactatcaactgtgtactctccaagtgaatgcgaataatcatttcctgtgagaattgtgggctagataaacgaatgaaatgctgttttatctatgtcatgtgtggaaatttagttaattttccggtctttttatgcattgagatgggtatgctgtttttttagttgggtcccatcatcttgagaattctttcaaatttccttttctttatcctatataaaggatagagaaggcgtatgcctaggtgcaccaaccctgaaagttttattctaattgcgggaatggtttgtaatttttgcttgttcaggttctttttcgtggcctttcttttttttccccttattttgcttagtctttcacagtccaatttttgggaagtagtatatcttagtttggtcctaaggcaccatgttgtactgcaggaaaaaaaagagtaattgtattctgttttttccttgattactatatccctgttttaattaattttgtgcctttgttgtttgatgttggaacttcaatgcccataattagtcatttgacttgttttgggttttgacgctatcttgagtgccataggaaactggtagaatttagtaataattttatatagactgaatgttgagcccaccacaaatggtttccttctgtacaagtatttaataactcaagcacaggaaacatcagatctctaatctaaaggttaacaatgggctcaagcaggagcagtagttcagctctatctgtatatttagaagggctggatctacctgtccaccagcttttaattttaccctggcagctggataacttcttgtctgttaatttcatttagtgctgtgttattttcttcttgttgttcaggatggatgcttttgaatttctggaatttcgtattttgttctatctctttatgaaatgacgttatggcacactttttctgcatattcttgatgaaaataattacctagtcatttttttagttgcaggtttgtctgggactttgagtacccatgcaattc

SEQ ID NO: 17 - DNA, 2315 bp, Oryza sativa
gttcaagatttatttttggtatttaatttacttgcttaagtcagatatattcccatcgttgcaggtttgtcacttagtattattattaagcgctctagcactaggactctggataaataagaaagtttattcacgaggctagagtagtaatcaataacataagcgtggtgtctaggtcagcggttatcttcatatgtagtgtgctccatggaaagtgaggtaggaggaaggtggtgacagtcccgtccgtcctttgtatccctccatgttcgggtatatcatagagctacaggctagacttagcttggcagactaggggagagccggtgctcgaagcaatccatgaggctttacatttaacataagttagtaaattaacccataggaatcatctctagactgaacctaccagtagttgtgcttggatataattatattcctacatatacatacacgttccctgcgattagatacccttggaatactctaaggtgaagtgctacagcggtatccgtgcgcttgcggatttatctgtgaccgtatcaaataccaacaggtagatacaaggaatcatctctcctatccattggtttatcatcttttaaaattatctcttgctctcctattgcctctgcaactgcggataggtgtttctcaacaatgaaggttgtgaagaatgctttgtgcaacaagatggatgacaagtatctcagccatagcctcatttgctttgtagaaaaggatatgtcggacacaatcactaagtatcaccgtggaaaggatgcactgtatgccctatctatatttaccatttagtaatatttatatggcttgtgctaactttatgttgtctttacaggcaataacattatttggaaggcatatctatatattactatttaagataatgtaatatctcaaagtttttataagctgcaatgaggtgagtttcacttagctttctaacttgttatgagttatagatgcatgccaccagtcattttttatcttgcatcagcccctgcctgttagaatatgtttctttgtctgggagtccatgtcaactagccaatttccaaatatatgaacaaaactatgtggcctttgtaacccaaatgagataaagactactctccatagaaatttagcaaacatggcactcaaagaaaatgtgttggatagtttcatcatgcatacaaaagcaacacttttgaactaccattccaaatcctttttgtaaattatctttgcttaacactacccctttgagcaaatgtggctttgtgcggaaaaaactcaaacttggtagggtagacatccatttatataattggatccatgtacataagttgttgagtacttcaagtacttacccttgtgatatacatctcaaatatattgaagaagagaagttctttttttgagagaggttgaagaagagaagtttgtccatagctgaagaggagttttatagtgtctagcttaccttgctgctgattgcatgtctaaaatgtcgtttaatttgggctataatgaaatattcaccaatatttctgctggtctattaaagtttaatagttactcgtaactcatttattttgggctataatttaatattcacctatgtttttgttagtctattttatttccctagtgtgcactagcttaaccccaaattagttttgaacacttaacctaaatgtgtctattatggtcagacactctctcacggcactctaacaaaaagtgaattttgttgttatgtttttgtcatgatctcacaagcaatgtacatgtacgtttctagagtgcaatcttatgctagcctgattgtgaatttagtgtagtttgttttctctttttgtagctacactaccaataacctattgtcctctagtcataccacgtaatcacaaggcaaatccctaactctcacctttaaaagcatgtctttattttcttgggtggcactaatacaaaatctttttcagcattcctatgtgcgatagcaagaaaacatggcataactcttgcttcactctaacaaaaaaaacacttttccaactttaaaacaatggtatctatgtgtttaatgatcaatcaagcatataatgacttacaagtttttacctatgccctttttgcatcatcttgtttgcaacagacaaactagatattcctttaggctataaacacatcagcatgataaagagattaggtaagtttgttatccctttttgcatatattctcgtctactccgtgtatataagcccctctcctccaactcgtccatccatcaccaagagcagtggga

SEQ ID NO: 18 - DNA, 1194 bp, Oryza sativa
ttgcatgccgtcgtcttaagcgtccgcgtgtgaaaatcggattttcgcatacggttgaaccggtcgcatgcaaagatcgcgatcttcgcagacgatttggcacatgcggttgcaccaaccgtatgcgaaaacccttctcgcccgtatgcaaaaaccatctttgttgtagtgtacggttcacaatggtttggatgggaaatcattgtgaaccaaaagtgatagactgatttcgacgagtgtttttttttaagtagtgccacaattttggtcatcatacgtcgtgtctaaaattgtaacttttgaaaaccaatttacattaaattaaatttataagactaaataaagacgatggtcattgaacaattgttgagaaaaatctacacacatgtgtgtccaacacaaatgtttacacatatactactatgttcatagtcgaagttagattttttttttccttaaagggaaagtctgttttcaaattttagacctcactccttccgtttcaaatatatcgtgtatttttttttctagggcaagcttttgaccaatgattactctattatgacacaatgttaaagggatagattcatattcaaaattactattataattataattttgtcatataaataatattttaagcaattgttagccaaaatctcgtcctaacgaaacaaaatacgccttatttttaaaaacacggagtatatccttaaatatttctctatccaatataaaaggtcaatcttttaaaattccgatcatcaataatttctcaaataattactttgaaataaaaaaacatatgcaaatttgtgtcgtcataatatccaatgaacttattcaaatttataaacttattttaattcaaaatttgatcattaattttttttttaaaaaaaaaccaaatcttatcataaacgtcaaatatatttttgatagtgggggcgataataccataaaactaacaacagaagagacatgatactactactgtaatcctaatacgtacgtacgtatacttctacgccggatgcataacttcagccttgtgagacacaacagttgctgcctagctcgtggtcgttggttttttcgctcgagaaaccactacgcgtaaaccgtgaagtatattatatatagccaactggtcttctcgcaaatccgcacatccctttctgcccctcgtcttct

SEQ ID NO: 19 - DNA, 1500 bp, Oryza sativa
gcaaagaaggccagtggcctttgcagctaagctagctagctagcccttcttcctctctttcctgctttccctttgccttctcctattaatcctctgcacctcacacagcagcagaaaacccaccaactggagctctcctttcctactccaagaaacgaaggtagagaaagaaagatcagatcagcttcaggaccaattttagctaggttatatatctctttgcgtgctaatgtgttttagttatctgggtgtgtgtagagttctttgttaaggcactgattcagctgcagtttagattcaagtttgtatgttctctctttgaggaaaagaaacccttttcctgtgcttcgagttcttgcaaagagaaactgtgatgcttggcttccagtttgatgcttctttgttcagattggaaattcttcctagcttctttctctatttatgtagcaaggattctttccggcccagtgatcctggtttcttttggaaggtttcagttttttcgttctttcttgaaatttctcttcttgccttaggcagatctttgatcttgtgaggagacaggagaaaaggaagaagctagtttcctgcggccgacctcttgcttctcactttgtgatgagttttctttggtcaattcttagctagatatgttaagatagttagttaagcaaatcgaaattgctagcttttccatgctttcttaaacatgattcttcagatttggttggttcttttttttcctttttgtggagacgtgctgttcttgcatcttatccttcttgattcatctacccatctggttctttgagctttctttttcgcttcttcccttcattatttcgagcaatctctgcacatctgaaagttttgtttcttgagactacttttgctagatcttgtttactcgatcactctatacttgcatctaggctcctttctaaataggcgatgattgagctttgcttatgtcaaatgatgggatagatattgtcccagtctccaaatttgatccatatccgccaagtctttcatcatctttttctttcttttttatgagcaaaaatcatctttttctttcaaagttcagcttttttctcttgttttacccctctttagctatagctggtttcttattccttttggatttacatgtataaaacatgcttgaatttgttagatcgatcactttatacacatactatgtgaatcacgatctcagatctctcagtatagttgaattcattaatttcttagatcgatcagcgtgtgatgtagtactgtaaatcactactagatctttcatcagtctcttttctgcatctatcaatttctcatgcaagttttagttgtttctttaatccggtctctctctcttttttaatcagctgagagtttgtgctgttctttaatcattaccagatctttcatcagtactctctcttctgcatctatcaaacttctcatgcaatgtttttgctgttctttgatctgatctct

SEQ ID NO: 20 - DNA, 1500 bp, Zea mays
agttttcgcttgtctattcaccctctataggcaactttcaattatgtaatcactttttttttcttttttctgtttaaaatctcagtttcaaacttccaattgattttgaatacgaggtttgggtttaaattcatattggaggcaaaaatcgaaagttccacgtgatgctaggttttatttcggttttctatctcctattgtttttcacgtttcaacttgattcaaattctagttttttttaacttaagcacaattaaatacaacataaaaacaacatggattcaagttctatttcaatttttattaactattatgttgtctagtctgttcaagcacataatacttataaatataaaattaaacgaaatcacatatttccacaaatcttgggtactacactcggagacgacgatggattccatctcaatttggatgttgattatagctctatttcagttgtcactgttgtcctaacacgccctattgtgcatgatagtgcacgtgctcaacgtaaaagaaaagagatcagtaacaagtagcagcactgtacaaggtaagccgtgattcaattaaaactgtttgagcaattcagttgctagatcgttccaccatcgataattcgatatgtacgatgatataaaaagagcccataagtttgtcttgaaaaggttgatcaaataatttaaattagatgataaaaaacatggaagatgtgggagtggacgacggctatgaagaatagtactatatcaggtttatacgtaaaatttatttttgaaatgtttttataatctgtttgaattgtattttttgcttaattatgtgattggatgttttttcatgaaatgtcgagttttattttaaataaaattctgtaaagagaagttgctgcgctgagaaaactataaatcgatagtaaaggctgtacgcaacgtttaagtccttgtttgaatgcgtatgaatctgagaaagttcagaatgattaaatcttttttatttaattttaatttgagagagattaagttctctccaattctctttaatttagacgtaatcgaacaagctggttgccaaactagatgagtacattttgtccactgccatagagccatcgactacaaaagtctagaacacagtggaaagcaccagacaacgcgcgaccaaaagggcccaggccccagcgccccagtccgggggttgtgttcgccgacctgtgcgtgcctgctcgtcacgtcacgtccctatttgcccgtcttcctcccctccagacccttctcgaacgccccttcgttctggatccaacggtcggtctctgccgggctcgaacgttctcgaaaccacgtcacccccgataaaaccccacgcacagcctcctcccttcctcaaccatcattgcaaaagcgaagcaagcaatccgaattctctgcgatttctctagatctcgaccacccctactagttttggttcctcctttcgttcgagagagcgtttctagtggca

SEQ ID NO: 21 - DNA, 1500 bp, Zea mays
caacttacaagcgatgaggccaagacgattagacgaatagctacagaacaagacaatgagagttcagcactcactttttgccagttccttctccttggcagcagccaggcgcttgagtttagcagcttgtgcaaatgtggacggcctacagcagacatacaggcaaagaagcgaggagtaatttgcagttggaaatcattcttcgatcaatagggaaactctgagtcacagcgaaaggaaggttaattgcctacgttgacaactgatcagcctccttgagaagttgcttgatttcaagccgcactttgatctgctcatcactaagtcctccgctctggatgacaaaagcacagaacgcatgagtggcaagtggaaacactagagcgaaataaatacaaaaccgcagactacaggctaacagatagggagaccgggaagacaaagactcgagcctgcattcaacagttacagtcgcctcggccaaaggttgagaaatttgcatcaaaatccaaactgtctagggccatgggaaatagttcctcggaatcagagttcaattcatggacgaaatagatggaactgatggtaggctactcttccgcccaatcagaattcacggaagatccaggtctcgagactaggagacggatgggaggcgcaacgcgcgatggggaggggggcggcgctgacctttctggcgaggtcgaggtagcggtagagcagctgcagcgcggacacgatgaggaagacgaagatagccgccagggacatggtcgccggcggcggcggagcgaggctgagccggtctctccggcctccgatcggcgttaagttggggatcgtaacgtgacgtgtctcctctccacagatcgacacaaccggcctactcgggtgcacgacgccgcgacaagggtgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 22 - DNA, 1500 bp, Zea mays
tctcataaaagcaataaaacaatatctcacaaaatacaagtggcaaacattatacaaacatacacatagtcagaaagtcacaactcaggaccttaaaaaatgaaactatccgattgaaaatacattgataacaattgaacactagaaaataatatcacaaatcaaactatggagcatataactagccatataactcttataatacaataataaaatcatcatatatttaaataaaacactagcaagtctaataacatatgactatagaatcaagatgtgtatgatgacatgacacttgcaattttatcatctcctactactcgacatagtcaatataattgatgtcctccttatctttaaagtttccatgcgaattataaatatatgtatgaagagtaatgattgataagaaactataaataagagtcacaatagttcaaacaactctaaactatatatcattagatagatcttgattttagaaaaataacgaaatcagtttcataattttctaagttaagatgaatttacaaagattagtttagatttaatattttttctgaaaaaataccgatttcggaaacgggcaaaagagatccaaactatttctgtttttttttaccgatttcatttccgtattttcggtaacggtttccggtttcgtatgaccctaaattttggtaaagtttcgaaaaaaaatattttaagaactgaaaattaacgttcctgttttcatccatactaatggctctttaccgctaaaatgttgcccacaatcattgagtaggtttagacgtgagagcaaacagtacaacattacgattcgcccttgcccaaatttacatgccttttccctacggaaacaacatagaatcaagttgacggggttacttacattgaagtggccaaactgatggtagctgtagatttggatgtatgttttctataaattagtcaaaattgagacaaaataaactgcaatttaaaactgaggaaatagtaaaaaaaaggtgaagaagggaggaagaggaaatcagaagcaaaaaatgggcaactttaggcccattatctcgatggtctcgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcgac

SEQ ID NO: 23 - DNA, 1507 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 24 - DNA, 1996 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctataaagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagg

SEQ ID NO: 25 - DNA, 1500 bp, Zea mays
cgagaatatatgttatcttcgtcgttagagaaatctagacagtatacaacaagatccacgtactacaggtaaacttttaggggtattgtgaacaagaggatgagtaaactctaaaagaacaaagctccaatgaaaatttaggtttttatgtggttagtcatagggcaagttgcaaacaggtgttgatctaaaaaggaagtagtagggaaatgtgaagtgtctttgcgaggaattggaaaatgaagatcacattttctttgggtgcatcatgggaagaaccatttgggactcttttaaggaggcctaagaatgccataaagtttgcaagatctttttgaagagtgtctacctataaacaatagtaaatatcatgtcaaaattttcatcttcgccattattctttaggagaatttagaatgttccgaataaaatatggatagaaaagaagttcccaaagtcatccaattttctacaaaatcttcaactttaagattgagagtgggtgttgtaaagttcttggaagatgagttgaaccccatggaggcgttggctaaagtactgaaagcaatctaaagacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagaggccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggattcacccgttcgcctctcaccttttcgctgtactcactcgccacacacaccccctctccagctccgttggagctccggacagcagcaggcgcggggcggtcacgtagtaagcagctctcggctccctctccccttgctccat

SEQ ID NO: 26 - DNA, 1500 bp, Zea mays
cgataagaacaatgttggacacaacttaagtctgttttacaacaatgtctctcaaaactatagttttacaatattatactttgcaattatcatgacaataatgtagtttcggtagctccaaaaatacagtagttttgagaaacattgtttagatacaatattataaatcatgtattagacaaaagatagccatgccattaaaactttgaattggactgtagttttttcaatactccaaaaatattatggtacctagaatacgatgtctagaaaacatattttttaaaatgcaaccaaacatcatatgacataaataatatagtatttttttgaaaaccatggtattacctaaaaactacagaatacttcattctgaaataggtcctaacaagttgcagcagctaggtcgtacatcagcaaatagctacttcatcaatctcagaataaacatattttatagatgagttaaactaaaaatatagaagaacaacgtacacgcgttgaatcacaacgtagcgcgatatccattcaactttttggaagtttttactgagcacaaattcgaaaatgggaagcgccacgtaacacgagcgctgggccaatttctgccagtgccagttatcccggcccacatccaatcctggggaagacgcgaacccggctccgcggcacgagttgtccgcacgtacggcacgtcggggctggctcgtccgcccgcgagtgggaggccactgtttcctctgcctcaccgggtcgtgtggcggaggggcgtggggccatggttcgcagcgcggggcgacgagcgcgctcctcctctcgcgcagcgccagcgccaccccgcaccgtggctttatatacacccctcctcccaaccctaccgaatcatcactaccaccgctctctcttcctctcctccatctctcaacgcctgaagctcaccgcacctcccctcctcgccgcggatcccccactactccggtaaccgtctctccattcaccctgcctgctgtctcgctagaatcgcctgcctctgccagcgccgtgacgcgggggcgcggtatggctctcccagatccgcctggcattgctcgctcgggtcgtgccaggccgatctgatctcgcatttgctgcgcgctcctcctgctgcggatcccaccggatctcgctggaatcggagcgcgcgtctctttgaaatgccgcagatctgcgtgcttgcgcgcgtgatctaagtccgggcctttcgttaacgaaatggtccgatctgtggtttggtggaggcaatgccatggtttttccccgtgaattttttttgctgattttaggagcttttttctactgtcctatgttagtaggacaaaaaaaaagaaacatagattagcttcaataggcgccttttagaacagattctgtacagcaactcgtggaaacaaatctgcttccttaatgatgttgcttgttttaacaaatgcggcatcgggcgagcttttctgtaggtagaaa

SEQ ID NO: 27 - DNA, 1694 bp, Artificial Sequence
  Synthetic construct cab5/hsp70 intron promoter
cacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtatggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcatacggtatttatttaagcacctgttgctgctatagggcacttgtattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgacgataagtcaagatcagatgcacttgttttaaatattgttgtctgaagaaataagtactgacagttttttgatgcattgatctgcttgtttgttgtaacaaaattttaaaataaagagttccctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcgg

SEQ ID NO: 28 - DNA, 1500 bp, Zea mays
tttaaatttggaacgtcgatccaacatctaacagaagcaccaattttacaaagaacccctttcaccttcctcacttggtgggacggttcttaatcaaattaactgcagccgctggtatacatgtacatgtgggcccgcctagcccggcacggcacaggcccacaaaaacacggtccacaaaagcacgacccacaaaagcacatatctaattatgggccgtgccgtgccagcacgtgtgcccagtcatcggcccacaattagttatgtgtgccaggccgacccaaatagcccaaaataccttaatatgccagaccggctcatatacatacaacagtaatacatcaacaaaacgtataaaatatatatatgaccaaaataaaactaagatgttttgtggatgcacattataaacctttggtcagaaagaaaaaaatattacaactagctcacaaaaaatatccagttctctgtttagtgtttaattgagtactatacatccatacagaataaatatacaatgatcatcatcactattcactatccatatctaggtattggttctcgatggcttattaaagctctagattctccaagttatgctagtcatgtgggctttgacagaccttagttaaatactgagtctatattttgtgggccttagttaaatgggtcgtggcaggccggcccgtgggcttgacttgaggcccaggcacggcccacaatgtgggccgtgccggcccatgcccacaattaggttgggcagtgccagatatgggccgtgccagaaattgtgtgctttgggccggcctattaggcacaacataaatgtacacctatagccgcatagccgctggatgtgagatgaatgtctcagatttaaaatgtgcacttgagcaccgtacctctttgaacaacagatatgttcctttaagattgatggtggaaaaaaattagtcagtacctcactgtatggcggcattgtttgattatttcagttcgcacccgttggaccttgctcattaaaaaagtttataccatggagtctttgcatgtagttgtgtagtaggggaagagtggcataggaggaatcacaacttcagctagcttctctagccttagggtatttttgtctttttgcagttcggtcttttcgcagccctgcgctgccccccctgtccgcctgtccctagacctgttttgcgtcggcggggaagacagttgacaggaaggacacgatcttcgtgtccgatgccgatcttcatgcgagcagcgagccactacgttgcgctgccagtgtcggctatggtatccaggcattcgttgtgcacgttgacgatgagctcgaagccggtccgggtgaacgcgagcagcacggtgaggtcaacgtcgtacatccgcacgtcgatgctgaggccagccagcagcggcatgacagattgcggcgtcaggagattgtgccagtaggtggcggggctgggggcagaccggcaggcgaggcct

SEQ ID NO: 29 - DNA, 1500 bp, Zea mays
caaaattttctattttttaaaaaatatgaattctagatttgggattgaacacatctaggctacaacgttgaattgatgaacaatagtgcttgttaataaattgctcacattcacattgtcgctcttacttcaaccatcatacatccatctacagtggtcacccatatttaatcctatggactaaagatgacagatgaacttctctcgttatatatatcactgtcctacatatatgagaaatgatatgtcctaaactcacctaaaaacaacaacatagtttaaatttaatcatagatgagcctacagaggtcgaacgtgatttggaaacatagctctattgttctctatctcatgcataaatatggtgcaatgaagaatattagggttatgatgtcgaaatctcactcgaactcgtgcctcatcataaatagcacactatcaattgttctatggctgttcaaatagggacaatcttgaaacaacatttctcacatgtaaaacgttgtgaagtatgccaactgaaacggatgacacatacacttcgtgaaccaatcgatattttacttgcttctatgttaaataatgttataatacaatattttattcaaatgctaaaacttattactagataaaaataaaatttaattatcttcaaaaactaaccaatagatattccatcataactacatttaccaaactaatatactaaaaaatataggataattactaaattaatcgtgcaataatcagtatttatgagattgataattttaaattttgtgggctacaaacaaaaattaaaacttacttttcaagttggagataagaacaatggtagacgtagctcgggatggtatggcgtcggtgcagacggttaccctttgtgcgaagtggcgcgggcacgagggtggggacttggtacatgcatgagagagaggaagaacgaaacaacttctcaaattaaagcatatgaaaatcacctaatttttgtctgtcggtggaaactaataactagtttttattatcttttttaataaggatccacgaaaattatttttgaccgatgaaaatcctggatcttcgtattatgtttcgccttttcccgactctttgcatgctagatttccatgcttggactaaaacgaagataataaaaccaatctatcattttcacacgatgtattcatacttgcaatagataaaccactactccgacgggatttgctttctgacctctgaaatcttggaaggattatgtgtctacacttctcgatcgaggggaaaaagtcgtagtaccaagttgtagttaaatttgtttcttcgatgacaaaacaaaggagaggggcccgcgcggcgcagcgcagcgcagttggctggttccggaacacgaaaaccaagcacactccaccagctgccatccaccgggttggatggagattacaatactcgaatagtcagccagccagccggcttgaacgtgcagttttcccctataaaacg

SEQ ID NO: 30 - DNA, 1500 bp, Zea mays
acacttgctctcttcgcgtggtcatttagcccccgaacattccaagaaaaaatagcacatttttgattcataaggtaaagactgccactccacttaacacagcacgctgccaccacacatggattagcaggagagcctgctgtaaaatcctaacaggagggagaacctccaaacaagggttcgccgagcaaaaacacagcccgaccacaaccgacaacctgaaagaacaacagagatacacaggcatgctgggggacctagaccagcgcccagaagtaataacgccagcggagatacaaccgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctggtcatcagtagtagtggccttttgcccttgcccctctctctcacctctcttttctcagccttgcttccgatggatcccatcccactgccatcctttctttcccttgcgcgcattgcctagccggccggccggcctgctattaaaccactttacccgccccctctcgctcacgctcgacgcagctcccttttccttgtttgcttattgcaagtctctgcaagaacctgctagagaggaacaaggtagagtagtatcgcttttttccatctaggttatctctttttacatgaaaaatttcagccgtatttcgttctccatcagtcctgcgataatatatacgcgcgtcttgtgtgatccggcatatgtatagttcctgctaactgatcgagatcgctctcgtttgtactttctccctttgaggaaagagtttccccttttctgtgcttcaagttcttgtaaggaaaaccatgcctgccagcttcttctgctacttgtatgatgattcttatttgcttattacttgatttccgttttttttcttgctttctatatgtatgtatctgggctgtcttcccctgcgtctcgttactgctaagctttggaaggtttcaactctttgtatacgatgaggtttctgctcctagtagcagatccgcgcatatgactagatgtttgaggaaaagaaaagggcaagacgctatatatatatgcagcacgcagtcgcacatatattcagttttccaatct

SEQ ID NO: 31 - DNA, 1500 bp, Zea mays
gattcgcgcgtgctggaactcgggattggattcatgcgtgctggaacttggaagtctggagtggactttggaagcctggattccagaacaagaactcaagaagtctaggagccgccgagcaggtagggaattagggaaataaagagaagaggcggctggcgttcgacgttccatcttcagtagaggcggctggcgtttcaactccctgtagtcgggccgcctgccaaaaaagcccacgaaggcaggaaatcaaaaaatctaggtcctaaacctagtcgcgcagaaccggctaatcgagcgactaatcgaccctaatcgtcgactagtcggacggccagggcgattaggtactctaatcgagtcggttgtgctaatcgagctctgctaaccgactagcccgaccgcgattagtcagatgacttgaaaacaaagatagagacatacttttttatattctttgcattgttttgtttctatccaaaactgctatttagaaattggaaaatctgcacattgaaaaatctaatggattagatatgttgatttgtttttattcacgagcataatcaaataaattagatttagaattggactgcacgcagtgaactactgaactgaactgtgttcaataatttaaatactcacggctgagccgtgagctgtaggctggagcacaagcacgagccagcaccgagcggcggagcactggagcagcaggcgagcagggaggcggccaggcgggagcagccagccagcaagcaggcagcagcggagcagcccacagccgagcgcccaagctggagctgctgcagagcctgcagcgtgccgctgcgcgccggcaggacaggagcggccgagcgggagtgcaggactgtggcctgcgggacgcggggatgggcggacggcgtagcgcttacagtccgcggacagcggactcacggtggcggctaagatagtgagaccgatgacctaatctctatttggaccggttcaaggttttgcccagttaatattggaccatattgggccttccgcccctgctcgcaagacacactgaacaaagaatccacacggctctccaaaagatagagagataattcacatgcttctctctctctgaaaaaaaggaacttgcatggttgacacggaaaacgtcattaaacgcgcacgtggctgcaaatgcaacgtaacagatccatcatctatccatccatagaatcagacggccacagaaggcaacgaccgtgtgcctgtccaccggcgcaggtggcccacagacgcccgtgcgattcatccgtctcggcccaccaaccacgggaggggccccagggccctccttagtccttacaaataccggcagcagcatcacccggccaccaccacccacccgttttatccacgcacggcgtcgaacaccccgcggtcgctcacgtgaggcgccaccccgcgcacccagtcagcgcccgcctccaccacccacccacacgacaaaaatccgcc

SEQ ID NO: 32 - protein, 796 aa, Escherichia coli
MAINNTGSRRLLVTLTALFAALCGLYLLIGGGWLVAIGGSWYYPIAGLVMLGVAWMLWRSKRAALWLYAALLLGTMIWGVWEVGFDFWALTPRSDILVFFGIWLILPFVWRRLVIPASGAVAALVVALLISGGILTWAGFNDPQEINGTLSADATPAEAISPVADQDWPAYGRNQEGQRFSPLKQINADNVHNLKEAWVFRTGDVKQPNDPGEITNEVTPIKVGDTLYLCTAHQRLFALDAASGKEKWHYDPELKTNESFQHVTCRGVSYHEAKAETASPEVMADCPRRIILPVNDGRLIAINAENGKLCETFANKGVLNLQSNMPDTKPGLYEPTSPPIITDKTIVMAGSVTDNFSTRETSGVIRGFDVNTGELLWAFDPGAKDPNAIPSDEHTFTFNSPNSWAPAAYDAKLDLVYLPMGVTTPDIWGGNRTPEQERYASSILALNATTGKLAWSYQTVHHDLWDMDLPAQPTLADITVNGQKVPVIYAPAKTGNIFVLDRRNGELVVPAPEKPVPQGAAKGDYVTPTQPFSELSFRPTKDLSGADMWGATMFDQLVCRVMFHQMRYEGIFTPPSEQGTLVFPGNLGMFEWGGISVDPNREVAIANPMALPFVSKLIPRGPGNPMEQPKDAKGTGTESGIQPQYGVPYGVTLNPFLSPFGLPCKQPAWGYISALDLKTNEVVWKKRIGTPQDSMPFPMPVPVPFNMGMPMLGGPISTAGNVLFIAATADNYLRAYNMSNGEKLWQGRLPAGGQATPMTYEVNGKQYVVISAGGHGSFGTKMGDYIVAYALPDDVK

SEQ ID NO: 33 - protein, 175 aa, Escherichia coli
MSTTNHDHHIYVLMGVSGSGKSAVASEVAHQLHAAFLDGDFLHPRRNIEKMASGEPLNDDDRKPWLQALNDAAFAMQRTNKVSLIVCSALKKHYRDLLREGNPNLSFIYLKGDFDVIESRLKARKGHFFKTQMLVTQFETLQEPGADETDVLVVDIDQPLEGVVASTIEVIKKGK

SEQ ID NO: 34 - protein, 603 aa, Escherichia coli
MNPQLLRVTNRIIERSRETRSAYLARIEQAKTSTVHRSQLACGNLAHGFAACQPEDKASLKSMLRNNIAIITSYNDMLSAHQPYEHYPEIIRKALHEANAVGQVAGGVPAMCDGVTQGQDGMELSLLSREVIAMSAAVGLSHNMFDGALFLGVCDKIVPGLTMAALSFGHLPAVFVPSGPMASGLPNKEKVRIRQLYAEGKVDRMALLESEAASYHAPGTCTFYGTANTNQMVVEFMGMQLPGSSFVHPDSPLRDALTAAAARQVTRMTGNGNEWMPIGKMIDEKVVVNGIVALLATGGSTNHTMHLVAMARAAGIQINWDDFSDLSDVVPLMARLYPNGPADINHFQAAGGVPVLVRELLKAGLLHEDVNTVAGFGLSRYTLEPWLNNGELDWREGAEKSLDSNVIASFEQPFSHHGGTKVLSGNLGRAVMKTSAVPVENQVIEAPAVVFESQHDVMPAFEAGLLDRDCVVVVRHQGPKANGMPELHKLMPPLGVLLDRCFKIALVTDGRLSGASGKVPSAIHVTPEAYDGGLLAKVRDGDIIRVNGQTGELTLLVDEAELAAREPHIPDLSASRVGTGRELFSALREKLSGAEQGATCITF

SEQ ID NO: 35 - protein, 213 aa, Escherichia coli
MKNWKTSAESILTTGPVVPVIVVKKLEHAVPMAKALVAGGVRVLEVTLRTECAVDAIRAIAKEVPEAIVGAGTVLNPQQLAEVTEAGAQFAISPGLTEPLLKAATEGTIPLIPGISTVSELMLGMDYGLKEFKFFPAEANGGVKALQAIAGPFSQVRFCPTGGISPANYRDYLALKSVLCIGGSWLVPADALEAGDYDRITKLAREAVEGAKL

SEQ ID NO: 36 - protein, 818 aa, Ensete ventricosum
MGLHNSNVTEKHTMSTDGASSPSRLLPRLLGVLLLIMGLALLAGGVKLSMLGGSLYYLLAGIGIALTGLLLLATHRAALGLYALVLFASTVWALWEVGLDWWQLVPRLALLFALGIVMLLPWFRRPLLRTQPAPLGTGALSIAVVLAGATALASQFTNPGEMVKTGQLDRDAVPGMASAAPSQADGDWNSYGRSAFGDRYSPLAQITPENAHKLVPAWTYRTGDIPGPNDPGETTAENTPLKVNGMLYVCTPHSQVIALDPDTGKEIWRFDPKISSQGAANFKGWAHMTCRGVSYHDDAVYASEQSPTGSASAAPVSAVCPKRIFVPTADTRLIALNADTGKMCEDFGDKGQVDLRANIGSFAPGGYYSTSPPAVTKNLVVIGGHVTDNVSTDEPSGVIRAFDVHTGKLVWNWDSGNPDDTTPLAEGKTYTRNSPNMWSMFAVDEKLGTLYLPMGNQMPDQYGGDRTPDSEKYSAGLTALDIDSGHVKWTFQFTHHDLWDMDVGGQPTLIDIKTADGVKQAVMASTKQGSIYVLDRTNGQPVVPINEIPVPQGAVAGDHTSPTQPKSDLNFMPPPLKERDMWGVTPFDQLICRIDFKSMRYDGPFTPPSLQGSIVYPGNFGVFDWGGISVDPVRQIAFVNPSYMAFKSKLIPAADIAKQGPRVSETEGVQPNKGAPYGVILEALLSPMGLPCQAPAWGYVAAVDLTTHKTIWMHKNGTVRDSSPVPIPLTMGVPSLGGTFTTAGGVSFLSGTLDQYLRAYDVKNGKQLWEGRLPAGAQTTPMTYTGKDGKQYVLVMAGGHGSLGTKQGDYVMAFKLPD

SEQ ID NO: 37 - protein, 226 aa, Quercus suber
MPSTVVADSAVSHYPPTPPSELVETEKAESTSNVSASASNPRGPQLKHIWVITGPAGCGKTSVAEHLHRTFSLPYLEGDTFHTAENVQKMANGHPLTDADRWDWLILLREQALSTLATASGVIVTCSALKRKYRDVVRVAAYHDPRICVHFVYLNAAEAVLLDRVRGRQGHYMKDSMVHSQFQSLEVPQADERDVMSVDAGGTSQEVLQLAVGVVRQVMGKEMPVA

SEQ ID NO: 38 - protein, 614 aa, Dorcoceras hygrometricum
MTLHPVLAEVTARIAERSRASRAAYLARIDAAHREGPHRAQLSCGNLAHGFAACGADDKAALRSGRAPNLGIVTAYNDMLSAHQPYERYPELIRRAARAAGVTAQVAGGVPAMCDGITQGRAGMELSLFSREVIAMATAVALSHDMFDGALMLGICDKIVPGLMIGALSFGHLPTVFVPAGPMPSGISNDAKTAVRQRYTEGKASREELMDAEAKSYHAPGTCTFYGTANSNQMLMEIMGLHLPGASFVNPNTPLRDALTEAAVRRLAEIATPGEQHMPLGRMFDERNVVNGVVGLHATGGSTNHLLHLVAVARAAGIALTLEDFDALSGVVPLMARVYPNGSADVNHFHQAGGMPFLIGSLVDAGLLHGDAHTVWGKGLDAYRAMPALDADGKLAWHEVGASGNLDVLRPATEPFRADGGLRVLEGNLGRAVIKVSSVPGDRLVIEAPAVIFHDQDEVKQAFDRGELDRDFVAVVRFQGPKAIGMPELHKLTPTLGVLQARGHRVALVTDGRMSGASGKVPAAIHVTPEAIDAGPLGKLRDGDLIRLDAIAGTLEAKVDAAEWAAREQVHADLSAHHVGMGRELFASLRNAVGSADAGATIFARHDRHHAADA

SEQ ID NO: 39 - protein, 371 aa, Ricinus communis
MRRRPRRLRSARCDRGRDPRPAAAALWPCFGRARGVRSGTGEYLRSPGHDRRATRSTRRRQGTVDHRTWGRRDAYCRRAGTFLHVPGGRGRRLRARAGCERRGHRRRIGPQARRCATAFGLRHTFRCQGPIRKHDGADTGQADHASRTGTLWGCSGLRRAESLMNAIDALMRGTPVIPVLVIDDVATARPIAEALAAGGLTTLEVTLRTPAALAVITEIAKVEGVTVGAGTVLNQHDMDASIRAGARFIVSPGLTEPLARAAITRDVPFLPGVASASDIMRGLDLGLDRFKFFPAEAAGGIKALRALAAPFRQCRFCPTGGITPATAADWLALDSVLCVGGSWLIPDDLEPAEIQVRARWASSLRRLSAGR

SEQ ID NO: 40 - protein, 609 aa, Acetobacter aceti
MIHTVLNEVTARIIERSRPRREAYLRRVSDAASNGPVRQALGCANQAHGFAGCGTPDKERLRDGPGPSLAIVTAYNDMLSAHRPYENYPELIRETARAVGGVAMVAGGVPAMCDGVTQGGAGMELSLFSRDVIALATGVALSHNTFDAACFLGICDKIVPGLVIGMLTFGHLPSVLIPAGPMPSGLSNPEKARIRQLYAEGKASRADLLKAEGEAYHAPGTCTFYGTANTNQMLVEVMGLHVPGAAFVQPDTPLREALTRFATRRVLSLTALGDEYLPVGRMIDERSFVNAIVALHATGGSTNHSIHLLPMAAAAGIILTWQDLADLAAITPLLCRIYPNGQADVNHFQAAGGTPFLIRELRDAGLLHADVETVMGRGLDAYVKEPFLEDGALAWRDAPTASGEETVLRPASRPFQPTGGLALLKGNLGHGVVKTSAVAPVRHVIEAPCRVFASQEAVQTAFKAGELHGDMVLVVRFQGPKANGMPELHKLMPPLGVLQDMGFRVALVTDGRLSGASGKVLSAIHVTPEALEGGPLAKLRDGDIVRVDAVKGSLDVLVPQAEWDARTDAVADLTSGHYGTGRELFSPFRHLVGRADHGASIFAFHEEPA

SEQ ID NO: 41 - protein, 604 aa, Hafnia alvei
MNPTVSRVTQRIIERSRDSRAAYLAKIEKARSKKVHRSQLACGNLAHGFAACGQDDKASLTSMIKSNIGIVTSYNDMLSAHQPYETYPEQIKAALHSVGAVGQVAAGVPAMCDGVTQGQDGMELSLMSREVIAMSAAVGLSHNMFDGALYLGVCDKIVPGLFMAAMSFGHLPTLFVPAGPMPSGLPNKEKVRVRQLYAEGKLGRDALLEAEAASYHAPGTCTFYGTANSNQMVIEVMGLHLPGSSFVQPDMPLRAALTDAAARQVTRMTETSGNYMPVGRMVDEKVIVNGIIALLATGGSTNHTMHLVAMARAAGIHINWDDFSDLSDVVPLMCRIYPNGPADINHFQAAGGVPVLVKELLKGGLLHEDVETVAGHGLSRYTQEPFLEEGKLVYRPGPEKSHDSNVIASFASPFESHGGTKVLTGNLGRAVMKTSAVPADNQIIEAPAVVFHSQHDVVPAFEAGKLDKDCVVVVRYQGPRAIGMPELHKLMPPLGVLMDRGLKVALVTDGRLSGASGKVPSAIHVTPEAYSGGLLAKVRDGDMVRVNGKTGELTLLVDEAELASRELVHPDLSELHDGSGREMFAALREQLSGAEEGASCIKFY

SEQ ID NO: 42 - protein, 618 aa, Proteus vulgaris
MNPKNHDSIQNDFVPLNETVKQVTERIIARSQATRRAYLNKIETAKSQTVHRAQLACGNLAHGFAACQVEDKNRLKNMVHNDIAIITAYNDMLSAHKPYEDYPQKIKTALHAVGAVGQVAGGVPAMCDGVTQGQDGMELSLLSRDIIAMSAAVGLSHNMFDGALYLGICDKIVPGLTMAALSFGHLPAIFVPAGPMTSGLPNKEKVRIRQLYAEGKVDRNALLEAEAASYHSIGTCTFYGTANSNQMVMEMMGLHLPGASFVHPDTPLRDALTDAAANQIVRLTENSGNYLPIGQLVDEKVIVNGIVALLATGGSTNLTMHLVAMARAAGIIINWDDFSELSRVVPLIARIYPNGPADINQFQAAGGIALIIRELLKKGLIHRDVNTVAGFGIERYTLEPWLNNGKLDWREGAISSLDKDVIADINTPFSSHGGTQVMQGNLGRAVMKTSAVPDENKIIEAPAVVFNSQHDIAAKFEAGELNKDCVVVVRYQGPQANGMPELHKLMPPLGVLMDKGYKVALVTDGRLSGASGKVPAAIHVTPEAVNGGLLSKVRDGDIIRVNGKTGELTLLVDEQTLNARQDEIPDLSANNTGCGRELFVNLRRHLSGAEQGACCIDF

SEQ ID NO: 43 - protein, 608 aa, Pseudomonas fluorescens
MHPRVLEVTERLIARSRATRQAYLALIRGAATDGPMRGKLQCANFAHGVAGCGSEDKHSLRMMNSANIAIVSSYNDMLSAHQPYEVFPEQIKNALREIGSVGQFAGGTPAMCDGVTQGEPGMELSLPSREVIAMSTAVALSHNMFDGALMLGICDKIVPGLMMGSLRFGHLPTIFVPGGPMVSGISNKEKADVRQKYAEGKATREELLESEMKSYHSPGTCTFYGTANTNQLLMEVMGLHLPGASFVNPNTPLREALTREAAHQVTRLTKQNGNFLPIGEIVDEKALVNSIVALHATGGSTNHTLHMPAIAMAAGIQLTWQDMADLSEVVPTLSHVYPNGKADINHFQAAGGMSFLIRELLEAGLLHEDVNTVLGHGLSQYTKEPFLDNGKLVWREGPTESLDENILRPVARAFSAEGGLRVMEGNLGRGVMKVSAVALENQIVEAPAMVFQDQQDLADAFKAGLLEKDFVAVMRFQGPRSNGMPELHKMTPFLGVLQDRGFKVALVTDGRMSGASGKIPAAIHVSPEAYVGGALARVQEGDIIRVDGVKGTLELKVDAAEFAAREPAKGLLGNNIGSGRELFGFMRLAFSSAEQGASAFTSALETLN

SEQ ID NO: 44 - protein, 607 aa, Zymomonas mobilis
MTDLHSTVEKVTARVIERSRETRKAYLDLIQYEREKGVDRPNLSCSNLAHGFAAMNGDKPALRDFNRMNIGVVTSYNDMLSAHEPYYRYPEQMKVFAREVGATVQVAGGVPAMCDGVTQGQPGMEESLFSRDVIALATSVSLSHGMFEGAALLGICDKIVPGLLMGALRFGHLPTILVPSGPMTTGIPNKEKIRIRQLYAQGKIGQKELLDMEAACYHAEGTCTFYGTANTNQMVMEVLGLHMPGSAFVTPGTPLRQALTRAAVHRVAELGWKGDDYRPLGKIIDEKSIVNAIVGLLATGGSTNHTMHIPAIARAAGVIVNWNDFHDLSEVVPLIARIYPNGPRDINEFQNAGGMAYVIKELLSANLLNRDVTTIAKGGIEEYAKAPALNDAGELVWKPAGEPGDDTILRPVSNPFAKDGGLRLLEGNLGRAMYKASAVDPKFWTIEAPVRVFSDQDDVQKAFKAGELNKDVIVVVRFQGPRANGMPELHKLTPALGVLQDNGYKVALVTDGRMSGATGKVPVALHVSPEALGGGAIGKLRDGDIVRISVEEGKLEALVPADEWNARPHAEKPAFRPGTGRELFDIFRQNAAKAEDGAVAIYAGAGI

SEQ ID NO: 45 - protein, 624 aa, Chlamydomonas eustigma
MSNVVLLQFGSFFISSRKMFSRMKAPSRTCDRSVNSGSSSRLPLVVKSVAAPESASESKPTVPAGLNKYSSRITQPKSQGASQAMLYATGLTEADMNKPQIGISSVWYEGNPCNMHLMDLAGEVKRGVEEIGLVGFRFNTIGVSDGISMGTDGMSFSLQSRDIIADSIETVMSAQWYDGNISLPGCDKNMPGTLMAMARLNRPSLMVYGGTIKPGYSASGSNATLDIVSAFQSYGAFSAGLIDEATRFDIVRNSCPGAGACGGMYTANTMASAIEALGMSLPYDSSIPAEDPLKKDECRMAGRYMLDLLKKDLKPRDIMTYKAFENAIVLTMALGGSTNAVLHLIAMAKSAGVSLKLDDFQRISDRVPFIADLKPSGKYVMEDVHKVGGTPAVLKYLHQKGYIHGDCLTVTGKTMAENLALVPELKAGQDVILPLEKPIKSTGHIQILYGSLAPEGSVAKITGKEGLYFRGKARVFDREEDMVEALSKDQASFKGTVIVIRYEGPKGGPGMPEMLTPTSAIMGAGLGKECALITDGRFSGGSHGFVIGHVTPEAQVGGPIALVQEGDMITIDAEKRVMDLEISEAELAARKAAWRAPPLKATSGTLYKYIKNVETASLGCITDS

SEQ ID NO: 46 - protein, 614 aa, Manihot esculenta
MQSTFVSPPANPINAAIPFYGALRQPHFFTRPSSLRVVAQSQTVVTSEPSSASTVAQKLNKYSSRVTEPKSQGGSQAILHGVGLSDEDLSKPQIGISSVWYEGNTCNMHLLRLSEAVKEGVKEAGMVGFRFNTIGVSDAISMGTRGMSYSLQSRDLIADSIETVMSAQWYDGNISIPGCDKNMPGTIMAMGRLNRPSIMVYGGTIKPGHFNGHSYDIVSAFQVYGEYVSGSISDEDRKNVVRNSCPGAGACGGMYTANTMASAIEAMGMSLPYSSSIPAENPLKLDECRLAGKYLLELLKMDLKPRDIISRQSLHNAMVVVMALGGSTNAVLHLIAIARSVGLELTLEDFQKVSDKVPFLADLKPSGKYVMEDVHKIGGTPAVLRYLLELGFLDGDCLTVTGKTLAENVLNCPPLSEGQDIIRSLESPIKKTGHIQILRGNLAPEGSVAKITGKEGLYFSGPALVFEGEEAMVAAISENPMNFKGKVVVIRGEGPKGGPGMPEMLTPTSAIMGAGLGKDCALLTDGRFSGGSHGFVVGHVCPEAQEGGPIGLVRDGDIITIDAQERRIDVDVTDQEMEERRKNWSPPPYKATRGVLFKYIKNVQSASRGCVTDE

SEQ ID NO: 47 - protein, 829 aa, Chlorella sorokiniana
MQALSATKGLRAGLAAQQRRPARRALAPACAAAPVKEVLEEGSKGFQTEVPTGLNKYSGHITQPKSQGASQAMLYATGLTQDDMNKPQVGISSVWWEGNPCNMHLNDLAAEVKAGVAEAGLVGLRFNTIGVSDGISMGTDGMSFSLQSRDIIADSIETVMSAQWYDANISLPGCDKNMPGTIMAMARLNRPSLMIYGGTIKPGYSKLGSNDTLDIVSAFQSYGGFTAGLINEDERCDIVRNSCPGPGACGGMYTANTMATAIETLGMSLPYSSSTPAEDPLKRVECRLAGRYVLEMLKKDIKPRDIMTRAAFENAMVIVMATGGSTNAVLHLIAMARSCGLDLTLDDFQRVSDRVPFIADLKPSGKYVMEDVHKIGGTPAVLKYLLERNLIDGSCLTVTGKTLAENLAACPGLKEGQQVILPLETPIKSSGHLQILYGNLSPQGAVGKITGKEGLVFEGKALCFDCEEDMLHALEQDQQQFKGSVVVIRYEGPKGGPGMPEMLTPTSAIMGAGLGKECALITDGRFSGGSHGFVIGHVCPEAQEGGPIALVQNGDTIRIDAETRSMDILNVDDAELQRRRAAWTAPPLKATSGTLYKYIKNVAPASMGCVTDAALSSKAAVQASFGVARPQRLSARRVGLVVRAAQVSSNDFKTGMTIEFDGAPYKVVEFLHVKPGKGAAFVRSKLKNFITNNTVEKTWRAGETVDLANVEKKETQFTYAEGDEYVFMDMTTYEETRVPRDEDWAKYMKEGMTVGVLLWNGKVISVEPPNTVELEVVETDPGVKGNTASGGSKPAKLETGAVIQVPLFIQQGERIKVDTRSDSYLSRAN

SEQ ID NO: 48 - protein, 598 aa, Quercus suber
MDPSNAKKQEQPEEPRYLDFPHSKPGSGLNKFSSTLTREHDFPAAQAMLYAAGVPDEHTMKTFPHVAIISVWWEANPCNTHLLELGKAVKHAVQGQEMLGWQHNTIGVSDGITMGGEGMRFSLQSREIIADSVETVLSAGHMDASVTLPGCDKNMPGVIMGMTRVNRPSIMVYGGSIMPGYSKTLKKPINITSALEAHGAYIYDNLKNPNDPTQTKDEILSDLERNACPGVGACGGMFTANTLATAIETLGLSLPGSASTPATSPAKMRECNKAAAALKICMEKQILPSHCLTKKSFENALVMMMALGGSTNGVLHLLAMAGTAGVDLTLDDFQRVSDRIPFIADLAPSGKHLMADLYEIGGVPSVQKLLIAAGLLDGTTITVTGKTLGENVESWPSLKQGQEIIRSLSDPIKATGHIEILYGNLAPKGAVAKITGKEGMRFVGKARVFNKEHELDDALNKGLIPRTENLVLIVRYEGPKGGPGMPEQLKASAAIVGASLKNVALITDGRYSGASHGFIVGHICPEAAAGGPIAIVQDGDLITIDAEKNRIDMDVDEAEIQKRFASWTPPKPTVTRGVLAKYAKLVGDASHGAMTDLF

SEQ ID NO: 49 - protein, 642 aa, Physcomitrella patens
MATSLLHSAAARGLHGLPSSFSLASSASLSSASQNQPLLILSKKLTRSKKEWRGMVVASSSAEGAVAAPPPQSVEAPAGSATDSAKLNKYSSRVTQPKAQGASQAILYGVGLSEEDMNKPQVGISSVWYEGNTCNMHLLHLSEAVKEGVKEAGMVGFRFNTVGVSDAVSMGTEGMCYSLQSRDLIADSVETVMGGQWYDANISIPGCDKNMPGVVMAMGRLNRPSIMIYGGTIKPGHFNGQTLDIVSAFQSYGEYVSGSISEDERMDVVRNSCPGFGACGGMYTANTMASAIEALGMSLPYSSSIPAEDPLKIEECKLAGKYILELLKRDLKPRDIITRKSLNNAMVVVMALGGSTNAVLHLIAIARSVGVELTLDDFQDVSDKTPFIADLKPSGKYVMEDLHKIGGVPAVLKYLLEKGLIDGSCMTVTGKTIAENLAECPPLSEGQKIIMPLENPIKKTGHLQILWGNLAPEGSVAKITGKEGLYFSGPARVFEGEEAMLDAITEDPQSLKGTVIVIRGEGPKGGPGMPEMLTPTSAVIGAGLGKEVALLTDGRFSGGSHGFVVGHICPEAQVGGPIGLVRDGDVITIDVEKRKINVDLTAQELADRKLLWRAPPYKASRGTLYKYIKNVKSASEGCVTDE

SEQ ID NO: 50 - protein, 600 aa, Quercus suber
MDPSNAEKQTQEQPEEARYLNFQHSKPGSGLNKFSSTLTREHDFPAAQAMLYAAGVPDEHTMKTFPHVAIISVWWEANPCNTHLLELGKAVKHAVQGQDMLGWQHNTIGVSDGITMGGEGMRFSLQSREIIADSVETVLSAGHMDASVTLPGCDKNMPGVIMGMTRVNRPSIMVYGGSIMPGYSKTLKKPINITSALEAHGAYIYDNLKNPDDPSQTKDEILSDLERNACPGVGACGGMFTANTLATAIETLGLTLPGSASTPAASPAKMRECNKVAAALKICMEKQILPSHCLTKKSFENALVMMMALGGSTNGVLHLLAMAGTAGVDLTLDDFQRVSDRIPFIADLAPSGKHLMADLFEVGGVPSVQKLLIAAGLLDGTTITVTGKTLGENVESWPSLKQGQEVIRSLSDPIKTTGHIEILYGNLAPKGAVAKITGKEGMRFVGKARVFNKEHELDEALNKGLIPRTENLVLIVRYEGPKGGPGMPEQLKASAAIVGASLKNVALITDGRYSGASHGFIVGHICPEAAAGGPIAIVQDGDLITIDAEKNRIDMHVDEAEVQKRFASWTPPKPMVTRGVLAKYAKLVGDASHGAMTDLF

SEQ ID NO: 51 - protein, 526 aa, Nicotiana tomentosiformis
MSKPQIGISSVWYEGNTCNMHLLKLAEAVKEGVQEANMVGFRFNTIGVSDAISMGTRGMCFSLQSRDLIADSIETVMSAQWYDGNIAIPGCDKNMPGTIMAMGRLNRPSIMIYGGTIKPGHFQGHTFDIVSAFQVYGEYVSGSVSDEERMNVVRNSCPGAGACGGMYTANTMASAIETLGMSLPYSSSTPAEDPLKLDECRLAGKYLLELLKMDLKPRDIITKKSLRNAMVMVMSLGGSTNAVLHLIAIARSVGLELTLDDFQKVSDAVPFLADLKPSGKYVMEDVHKIGGTPAVIRHLLELGYLDGDCMTVTGKTLAENAKLFPSLAEGQQIIRPLSNPIKETGHIQILYGNLAPDGSVAKITGKEGMYFSGPALVFEGEEAMIAAISEDPLSFKGKVVVIRGEGPKGGPGMPEMLTPTSAIMGAGLGKDCALLTDGRFSGGSHGYVVGHICPEAQEGGPIGLVQDGDIITIDIQKRKMDVQLSDEEFEQRRKNWTPPAYKADRGVLYKYIRNVQSASKGCVTDE

SEQ ID NO: 52 - protein, 616 aa, Micractinium conductrix
MQALSSKTCGLPASRAAGRRPSRVQRPATVCQGAAAPVQEVLEAGSQGFNGEIPKGLNKYSGQITQPKSQGASQAMLYATGLTEADMSKPQVGISSMWWEGNPCNMHLNDLAAEVKAGVADAGLVGLRFNTIGVSDGISMGTDGMSFSLQSRDLIADSIETVMSAQWYDANVSLPGCDKNMPGTIMAMARLNRPSLMIYGGTIKPGHSQLGSNATLDVVSAFQSFGGYAAGLITEEERCDIVRNSCPGPGACGGMYTANTMASAIEALGMSLPYSSSTPAEDPLKRLECRMAGRYVLELLKKDLKPRDIMTLAAFENAIVLIMATGGSTNAVLHLIAMARSCGLELTLDDFQRVSDRVPFIADLKPSGKYVMEDVHLVGGTPAVLKYLHQNNLIDGSCMTVTGRSLADNLATCPGLKEGQQVILPLNKPIKSSGHLQILYGNLSPDGSVGKITGKEGMVFEGHALCFDCEEDMLAALEKDQEQFKGSVVVIRYEGPKGGPGMPEMLTPTSAIMGAGLGNDCALITDGRFSGGSHGFVIGHVCPEAQEGGPIALVQDGDTIRIDVEKRSMDILNVDDAEMQRRRDAWTAPPLKATAGTLYKYIKNVASASLGCVTDM

SEQ ID NO: 53 - protein, 613 aa, Nicotiana attenuata
MQAQLLSPPTYHPTPTTPTTTIFPKCRPHFKKPPTFTIKATTTTSTPPPPPQTTTQKLNRYSSRITEPKSQGGSQAILYGVGLTDEDMSKPQIGISSVWYEGNTCNMHLLKLAEAVKEGVQEANMVGFRFNTIGVSDAISMGTRGMCFSLQSRDLIADSIETVMSAQWYDGNIAIPGCDKNMPGTIMAMGRLNRPSIMIYGGTIKPGHFQGHTFDIVSAFQVYGEYVSGSVSDEERMNVVRNSCPGAGACGGMYTANTMASAIETLGMSLPYSSSTPAEDPLKLDECRLAGKYLLELLKMDLKPRDIITKKSLRNAMVMVMALGGSTNAVLHLIAIARSVGLELTLDDFQKVSDAVPFLADLKPSGKYVMEDVHKIGGTPAVIRHLLELGYLDGDCMTVTGKTLAENAKLFPSLAEGQQIIRPLSNPIKETGHIQILYGNLAPDGSVAKITGKEGMYFSGPALVFEGEEAMIAAISEDPLRFKGKVVVIRGEGPKGGPGMPEMLTPTSAIMGAGLGKDCALLTDGRFSGGSHGYVVGHICPEAQEGGPIGLVQDGDIITIDIQKRKMDVQLSDEEFEQRRKNWTPPAYKADRGVLYKYIRNVQSASKGCVTDE

SEQ ID NO: 54 - protein, 604 aa, Nostoc sp. 3335mG
MTVRQSIRDVTDRIAARSRDSRRDYLKRIEAAREAGVYRSSLSCGNLAHGFAACTPAEKAALAGNKTLNLGIVTSYNDMLSAHQPYQFYPDIIKEAAREMGATAQVAGGVPAMCDGVTQGQPGMDLSLFSRDVIAMATAIALSHNMFDAAVYLGICDKIVPGLVIGALTFGHLPAVFIPAGPMPSGIPNDEKSKVRQLYAEGKVGRAELLEAESKSYHSAGTCTFYGTANSNQMLMEIMGLHLPGASFVNPGTPLRDALTREATKRALSLSAQGNDYTPIGHIIDEKAIVNGLVGLHATGGSTNHTMHLIAMAAAAGLQVTWDDMSDLSDATPLLARVYPNGVADVNHFHAAGGMGFLIRELLDEGYLHEDVKTVWGAGLHNYTVEAKLIEDKLAFEPAPAESALPKVLTSARTPFQQTGGLKLLTGNLGRSVIKVSAVKPEHRVVEAPARVFHGQDGLIAAFKAGELTGDVIAVVRFSGPKAIGMPELHKLTPPLGILQDRGFKVALLTDGRMSGASGKVPAAIHMTPEAIDGGPISKIRNGDLIRLDANEGTLTFLGDEKEFFSRTPAIEDLRPQQFGMGRELFAGFRNLVGVADRGASVFG

SEQ ID NO: 55 - protein, 609 aa, Nostoc sp. 3335mG
MTSLHPAVHAVTQRIVERSKPARERYLDLIERQRDAGVDRSRLGCANLAHGFAAAGEDKATIRAGAAMNIGIVTAYNDMLSAHQPYYRYPEQMKLWAREVGATAQVAGGVPAMCDGVTQGYAGMELSLFSRDTIALSTAIGLSHGMFEGAAMLGICDKIVPGLLIGALRFGHLPMLLIPAGPMPSGLANKEKQRIRQLYAEGKCSRDELLESEAASYHGAGTCTFYGTANSNQMMMEMMGLHVPQSAFVNPGTKLRQALTRAAVHRVAEIGWDGDDYRPLGKVVDEKAVVNAAIGLLATGGSTNHAIHLPAIARAAGIVIDWEDIDRLSSIVPLLARVYPNGSGDVNHFQAAGGLAYITKTLLEHGLLHGDILTIAKGGFADYTADPRLDGEDLVWEPAPAEPLDASMVRPASDPFQADGGMRLVKGNLGRAIFKTSAVDPSRWTIEAPARVFSDQLQVLEAFKAGELDRDVIVVVRFQGPRANGMPELHKLTPPLGVLQDKGFKVALVTDGRMSGASGKVPAAIHVSPEVLGGGPLGKLRDGDMVRLSAEEGSLEALVDFDEWEARQIADAPPPALGTGRELFAFMRNGADDAEKGGSAMLAAMDAIV

SEQ ID NO: 56 - protein, 606 aa, Nostoc sp. 3335mG
MALNATIAAVTDRIIEASRTKRAAYLALIDRERESGVDRPMLGCANLAHAYAGTDEDRDAMRPANRMNIGIVTAYNDMLSAHATYYRYPEQMKVWAREAGVTAQVAGGVPAMCDGVTQGFPGMELSLFSRDTIALSTAVALSHGTFEGAALLGICDKIVPGLLMGALRFGHLPMVLIPGGPMRSGIPNKQKAAVREAYAEGKVGREELLDAEIAAYHGKGTCTFYGTANSNQMMMEAMGLHVPDAAFANPGTKLRQELTRHAVHRLAEIGWDGDDYRPIGRVVDEKAIVNAAIVLLATGGSTNHLIHLPAIARSAGIIIDWEDFNQLSQVVPLLTRVYPNGSADVNGFEDAGGPSFVIRELLAGGLMHGDVLTIARDGMAAYGRKSTIENDQLVWKEHGAVSGDDTIVRTVADPFNDEGGFRILQGNIGRACIKVSAVDRDRWVIEAPCRVFSDQASVQAAFKAGELDRDVVVVVRHQGPRANGMPELHKLTPPLGVLQNRGYRVALVTDGRMSGASGKVPAAIHLSPEALGGGPIGKLRDGDIVRLDADANTLEALVPADEWAAREHAPVPPEADGTGREIFAMMRQGASEAEAGGSAMLAAAGL

SEQ ID NO: 57 - protein, 551 aa, Cyanobacteria bacterium UBA11991
MRSDNIKKGIAHTPHRGLLMAAGVSRKNMDAPFIGIASSFTDLIPGHAGMRDLERRIEKGIHTGGGQAFIFGVPAICDGIAMGHNGMRYSLPSRDLIADCVETVAQGHQLDGLVLLTNCDKITPGMLIAAMRLNIPSIIVTAGPMLEGESRCKKLTMIKGSFEAVGKYRNGEIDKDRLLELEEESCPTFGSCQGMYTANTMACLTEVMGMSLPLCATSSAVSSKKRRIAFDSGVQIVNLVKNDIKPSDIIDKETIENAIIADLALGGSTNSVMHLLALANSGGIDLKLEDFDRWGNTVHQILKLDPAANPTMTEFHYAGGVPALMKNLIEANIGIKNKNTVSGLSTFEISKNAFVDKNLIKTYDNPVNTKPGLGILHGNIAPDGCVTKISGIDESCFEFEGKAVCFDCEEDAMSALENDEIKDGDVIIIRYEGPKGGPGMREMLAPTSLLVGKDLGKTVALITDGRFSGGTRGICIGHVCPEAANGGNIALIENGDKIKIDINNRRIELLVSEDELNERRKHLKPFENKIKSGYLAKYARTVQDASHGAIV

SEQ ID NO: 58 - protein, 552 aa, Cyanobacteria bacterium UBA9971
MKSDKITEGLDRAAHRGLLYSLGLTKKQLKAPFIGIASSFSDLVPGHSGMRDLERFIEKGVHSAGGHSFIFGVPAICDGIAMGHEGMRFSLPSRDLIADCVESVASAHSLDGLVLLSNCDKITPGMLMAAARLNIPCIVVTAGPMMDGKCENQTLTLIRGTFEAVGQVKAGKITEEELEKYEINSCPGIGSCQGLYTANTMACLTEVMGMSLPGCASALAVSAKKRRIAFDSGFRIVELVKEKILPLDIITKDSVRNAIIADLALGGSTNSILHLLAIANEAGLDISLKDFEDLSSKIPQIIKLDPSSNVTMTDFDNAGGISGALKNLIQKDIGISNTKTLSGFTIKEIAENAWVDTEVIRPVDNPITSNPGLAVLYGNIVPEGAVIKISGVDPECLKFEGTARIFNSEDDVMAAINTDVVQSGDVVVIRYEGPKGGPGMREMLAPTSSIVGKGLGKSVALITDGRFSGGTRGLCIGHIAPEAAAGGAIAYLKDGDKIKIDILARELNVDISDEELAKRKQEIQLLPQKITKGYLSRYSKTVTSASKGAISC

SEQ ID NO: 59 - protein, 551 aa, Cyanobacteria bacterium UBA10660
MRSNCIKEGIARTPHRGLLMATGVSKKNMDVPFIGIASSFTDLVPGHIGMRDLERQIEKGIHSGGGQAFIFGVPAVCDGISMGHSGMRYSLPSRDLIADCVETMAEAHQLDGLVLLSNCDKITPGMLIAALRLNIPTIIVTAGPMLDGESRCEKLTMIKGSFEAVGKFRSGVIDKDRLLELEEESCPSAGSCQGMYTANTMACLAEVMGMSLPLCGTAAAVSSKKRRIAFDSGMQIVELVKNDVKPLDIVDKDTLRNAIICDLAMGGSTNSFLHILALANAGGIDVTLDDFEELSYKIHQILKLDPATSFTMTDFHKAGGVPALLKTLQDANVGLVDKNTVSGLKISEITKNAYVNTDLIHPYSEPFTTKPGLGILHGNIAPDGCVTKISGIDSSCYEFEGVAKTFDSEELAMKALEDDKIKAGDIIIIRYEGPKGGPGMREMLAPTSLLVGKGLGTTCALITDGRFSGATRGICIGHVCPEAAEGGTIALIEDGDKICIDINKRTMNLLVSDEELKKRREKLKPFELKCNSGYLAKYAKNVQDASHGAIV

SEQ ID NO: 60 - protein, 563 aa, Cyanobacteria bacterium UBA9579
MRSDRIKKGIDRAPHRALLHSVGVSKDQLDKPFIGIASSFSDLVPGHSGMRELERAIEKGIHSGGGHSFIFGIPAICDGIAMGHEGMRFSLPSRDLIADSVESVASAHALDGLVLLTNCDKITPGMLMAAVRLNIPTIVVTAGPMSDGYCEGKDLTFIKGTFEAIGQYRTGQITEEELSKMEMNSCPGIGSCQGLYTANTMACLTEVMGMSLPGCAAASAVSAKKRRLAFESGIKIIDLVRNDVTPRTIITKESIRNAIIADLALGGSTNSVLHLLAIASEAEVDVTLNDFAQLSHQIPQIIKLDPSSTVTMTDFEKAGGIPGTLKNLLKGDSRLVNTKGVSGLALQDIAEKAWVDSNVIHNINEPITSNPGLAVLYGNLAPEGSVVKISGVAPECLKFEGIAKIFNSEEDAMIAINSDKIVAGDVIVIRYEGPKGGPGMREMLSPTSAIVGRGLGNQVALITDGRFSGGTRGLCIGHIAPEALAGGTIGLLQDGDAICIDIENRTLEVLLTEEQLRARKQSWKALPPKINKGYLARYARTVSSASLGAVTSFCPADLESISH

SEQ ID NO: 61 - protein, 560 aa, Planktothricoides sp. SR001
MPENLRSQVVTQGTQRSPNRAMLRAVGFGDDDFTKPIVGVANGYSTITPCNMGLDDLAKQAQAAIRDAGGMPQVFGTITISDGISMGTEGMKYSLVSRDVIADSIETVCNGQSMDGVLAIGGCDKNMPGAMIAIARMNIPAIFVYGGTIKPGNYEGRDLTVVSAFEAVGQYSAGKISEAELLGVERNACPGAGSCGGMYTANTMSSVFEAMGMSLMYSSTMAAEDREKADSTADSAKVLVNAIRNQILPRQILTRKAFENAISVVMAVGGSTNAVLHLLAIAHAAGVKLTLDDFTTIRDRVPVLCDLKPSGQYVITQFHQAGGVPQVMKMLLEHGLLHGDCLTITGKTIAEQLADIPSEPPANQSVIRPWDQPMYAQGHLVVLKGNLATEGAVAKISGVKNPKITGPARVFESEEECLQAILDGKIKAGDVVVVRYEGPKGGPGMREMLAPTSAIIGAGLGDSVGLITDGRFSGGTYGLVVGHVAPEAAVGGAIALVKEGDTITIDATTRQLTLEISDRELAERRANWQPPKPRYTAGVMAKYAKLVASSSVGAVTDLNL

SEQ ID NO: 62 - protein, 561 aa, Planktothrix paucivesiculata PCC 9631
MSDNLRSQAITQGTQRTPNRAMLRAVGFGDQDFIKPIVGIANGYSTITPCNMGLNTLALRAETALKQAGAMPQMFGTITVSDGISMGTEGMKYSLVSREVIADSIETACNAQSMDAVLAIGGCDKNMPGAMIAISRMNIPAIFVYGGTIKPGHYNGKDLTVVSAFEAVGQYSAGTIDETELIEVERRSCPGAGSCGGMYTANTMSSAIEAMGMSLMYSSTMAAEDEEKAESTEKSAYVLREAIKNQILPRQIITRKAFENAIAVIMAVGGSTNSVLHLLAIANAIGVELNIDDFEAIRAKVPVLCDLKPSGRYVATDLHKAGGIPLVMKMLLEKGLIHGDALTITGKTVAEQLADVPSEPSADQDVIRPWNNPMYAQGHLAILKGNLATEGAVAKITGVKNPVITGPARVFESEEECLDAILAGKISAGDVIVIRYEGPKGGPGMREMLAPTSAIIGAGLGDKVGLITDGRFSGGTYGMVVGHVAPEAAVGGNIGLVKEGDTITIDAHQRLLQVNISEEELAQRRQTWQAPKPRYTKGVLAKYAKLVSSSSIGAVTDLNLG

SEQ ID NO: 63 - protein, 561 aa, Oscillatoriales bacterium UBA8482
MSDNLRSQTITQGTQRTPNRAMLRAVGFGDQDFVKPIVGIANGYSTITPCNMGINGLALRAEAALKQAGAMPQMFGTITVSDGISMGTEGMKYSLVSREVIADSIETACNAQSMDAVLAIGGCDKNMPGAMIAISRMNIPAIFVYGGTIKPGHYNGKDLTVVSAFEAVGQYSAGKIDDTELIEVERHSCPGAGSCGGMYTANTMSSAIEAMGMSLMYSSTMAAEDEEKAESTEKSAYVLREAIKNQILPRQIITRKAFENAIAVIMAVGGSTNSVLHLLAIANAIGVELTIDDFEEIRAKVPVLCNLKPSGQYVATDLHKAGGIPLVMKMLLEKGLIHGDALTITGKTVAEQLADVPSEPSPDQDVIRPWNNPMYAQGHLAILKGNLASEGAVAKISGVKNPVITGPARVFESEEECLDAILAGKISAGDVIVIRYEGPKGEPGMREMLAPTSAIIGAGLGDKVGLITDGRFSGGTYGMVVGHVAPEAAVGGNIGLVQEGDSITIDARQRLLQVNISDEELAGRRQNWQAPKPRYTKGVLAKYAKLVSSSSIGAVTDLNLG

SEQ ID NO: 64 - protein, 214 aa, Acetobacter aceti
MSQGHIQDGLEHILRLAPVVPVVIVESVAEAVPLARALVKGGLKAIEITLRTAAGLDAIRAVAAEVEGAVPGVGTVLTPAQYRQAAEAGARFAVSPGATPRLLDAAEDAGLAPLPGIATASEAMALIERDYRFAKFFPAEAAGGAPFLASIASPLPQLTFCPTGGITPDIAPRYLKLPNVICVGGSWMVKRSVIEAQDWDAITRAAEQAAAITG

SEQ ID NO: 65 - protein, 247 aa, Hafnia alvei
MQSINFYQGRVAINVLAKNIENARAVYDAAEGHAVIGVLSAQFDNVAQGIEEVKRWQLQVPSISVGLGAGDPAQFYKAAMIAAATGAAHVNQTFTGCGFAAGALAQAGQSHTHINALVSPTGKMGQVQISTGERSAKGDKAIVSCDSAVNMMLDMGAHAAKFFPMGGERSLEELKTLAESAARNGMTLIEPTGGIDLDNFGIILQTCLQAGMPRVMPHVYTSIIDPKSGETRPEDIKQLMDMIKAIV

SEQ ID NO: 66 - protein, 213 aa, Hafnia alvei
MKNWKVSAESIMTAGPVVPVIVIKKIEQAVPLAKALVAGGVRVLEVTLRTECAVEAIRLIAKEVPEAIVGAGTVTNAKQLADVTEAGAQFAISPGLTADLLKAATAGSIPLIPGISTVSELMLGMDHGLKEFKFFPAEANGGVKALQAIAGPFSHVRFCPTGGINPQNYRDYLALKSVLCIGGSWLIPNDALDNGDYARITELAKEAVAGAKA

SEQ ID NO: 67 - protein, 213 aa, Proteus vulgaris
MDHWNTSAESVLKSGPVVPVIVINHIDDAVPVAKALVAGGVKVLEVTLRTECAIEAIRRIAKEVPEAIVGAGTVINPQQLAQVTEAGAQFAISPGLTEALLKSAVAGTIPLIPGISTVSELMLGMSYGLNEFKFFPAEANGGVKALKAIAGPFSKVRFCPTGGISPENYRNYLALESVLCIGGSWLVPNDAVKAGDFQRITELAKEAVAGTQR

SEQ ID NO: 68 - protein, 213 aa, Proteus vulgaris
MTQSIVDTLSSLKVIPVIQINRAEDAIWLGEILTQNQLPIAEITFRTPAAVKTIKLMHEHFPELILCAGTVLTAQQADMAKEAGASFVISPGYNPSTVDYCLNNGIDIVPGINNPSQIEVALERGLTLVKFFPAEASGGIKMLKALAAPYSQMQFMPTGGISLNNVSDYLAIPQVVACGGSWIATADTIDKQDKETIVNHIQNIHSLLKTDKG

SEQ ID NO: 69 - protein, 221 aa, Pseudomonas fluorescens
MTTPSPTVSMADKVALIDSLCAKARILPVITIAREQDVLPLADALAAGGLTALEVTLRSQFGLKAIQILREQRPELVTGAGTVLDRNMLAAAEAAGSQFIVTPGITRDLLEASVESPIPLLPGISNASGIMEGYGLGYRRFKLFPAEVSGGVAAIKALGGPFGEVKFCPTGGVGPANIKSYMALKNVMCVGGSWMLDPEWIKNGDWARIQECTAEALALLD

SEQ ID NO: 70 - protein, 208 aa, Zymomonas mobilis
MRDIDSVMRLAPVMPVLVIEDIADAKPIAEALVAGGLNVLEVTLRTPCALEAIKIMKEVPGAVVGAGTVLNAKMLDQAQEAGCEFFVSPGLTADLGKHAVAQKAALLPGVANAADVMLGLDLGLDRFKFFPAENIGGLPALKSMASVFRQVRFCPTGGITPTSAPKYLENPSILCVGGSWVVPAGKPDVAKITALAKEASAFKRAAVA

SEQ ID NO: 71 - protein, 214 aa, Leptolyngbya valderiana
MTDAPDLALLDRLMTRAPVIPVLTLDDPGRAVDLARTLVDAGLDVLEVTLRTDQALAAIRRIADEVPEALVGAGTCIAPSDLDRVADAGGQFAISPGLTPPLCERALGIGLPLLPGVATASEVMTGLALGWERFKLFPAKACGGVALLRSFSGPFPGVCFCPTGGIGAQDFRDFLALPNVTCVGGSWMVPADRLAASDWVSIGRLARACLDPPA

SEQ ID NO: 72 - protein, 204 aa, Nostoc sp. 3335mG
MADVDALMRKAPVIPVLVIDRLEDAVPIAESLVAGGLPLLEVTLRTPVALDAIRAMAQVPGAVVGAGTVLNGRQLHDAQGAGATFIVSPGLTAPLAKAAAAADIDFLPGVANPADIMTGLDLGLTRFKFFPAEAVGGLKALKAYASVFRDVRFCPTGGITAETAPQWLAEPAVLCVGGSWLVPSGAIDEAAIRQRAEAAAGLRA

SEQ ID NO: 73 - protein, 211 aa, Nostoc sp. 3335mG
MPQDANAIRSILSLAPVVPVIILDDVSQARPLAEALVAGGLPVLEVTLRTPNALKVMEEMAKVTGAIVGSGTVRHAAHMQASVDAGCRFMVSPGASPRLLDAADDIAIPLLPGIGTPTEAMTAAERGYSFLKFFPAEALGGAPVLKAFASPLPDITFCPTGGIDPAKAKTYLGLPNVICVGGSWIMPADALASGDFARIEALAKEASALRG

SEQ ID NO: 74 - protein, 175 aa, Nostoc sp. 3335mG
MTVTISDIMQTSPVIPVLVVHDAAEARPMAEALVKGGLRVLEVTLRTPAALEAIAEMKKVDGAIVGAGTVVNADQFRQAMDAGSEFIVSPGLTETIARPIIDSKVPFLPGIANAGDIMRGLDLGLTHFKFFPAEASGGLKALKALAAPFYQAKFCPTGGITEASAPEWLAFDPVL

SEQ ID NO: 75 - protein, 222 aa, cyanobacterium endosymbiont of Rhopalodia gibberula
MSSNSSRNPSLVWQDLLRQHRAIAVIRTNNLNHGLDMAKAVSAGGIRIIEITWNSDRPVQLIRQICEELPNCTIGTGTILNLEQLQEAIAAKVQFCFTPHVNPTLIQTAINAEIPIIPGALSPTEIITAWQLGASCVKVFPVQAIGGVPYIEGLQGPLGSIPLIPTGGVTLDNAADFIQAGAIAVGLSGQLFPENAIDNKNWEVITQKAQILLQQLKPFSSI

SEQ ID NO: 76 - protein, 223 aa, cyanobacterium endosymbiont of Rhopalodia gibberula
MISSNSSRNPSLVWQDLLRQHRAIAVIRTNNLNHGLDMAKAVSAGGIRIIEITWNSDRPVQLIRQICEELPNCTIGTGTILNLEQLQEAIAAKVQFCFTPHVNPTLIQTAINAEIPIIPGALSPTEIITAWQLGASCVKVFPVQAIGGVPYIEGLQGPLGSIPLIPTGGVTLDNAADFIQAGAIAVGLSGQLFPENAIDNKNWEVITQKAQILLQQLKPFSSI

SEQ ID NO: 77 - protein, 215 aa, Coleofasciculus chthonoplastes PCC 7420
MSISNQSWLTLLQDYRTIAVIRASDQSVGRQMAQAVAAGGIKLIEITWNTDAAADLIRHLRQALPDCTIGTGTLLNLEQVKQAIEAGAEFLFTPHTNITLINTAVNAGVPIIPGALSPSEIVTAWDAGASCVKVFPIQAVGGVSYIKALQGPLGHIPLIPTGGVTFANAKDFITAGAIAVGLAGDLFPTSLIASGDWHGITQRAKKLRSQFSGDP

SEQ ID NO: 78 - protein, 213 aa, Coleofasciculus chthonoplastes
MSNQSWLTLLQDYRTIAVIRASDQSVGRQMAQAVAAGGIKLIEITWNTDAAADLIRHLRQALPDCTIGTGTLLNLEQVKQAIEAGAEFLFTPHTNITLINTAVNAGVPIIPGALSPSEIVTAWDAGASCVKVFPIQAVGGVSYIKALQGPLGHIPLIPTGGVTFANAKDFITAGAIAVGLAGDLFPTSLIASGDWHGITQRAKKLRSQFSGDP

SEQ ID NO: 79 - protein, 211 aa, unclassified Calothrix
MADDWLSILQKNRAFAVIRAPSLEIGRQMALAVAASGMRLIEITWNSDKAVDLITKLRCELPDCIIGTGTVLNLQDLHSCIDCGAQYIFTPHVDPELIHAAVAQNIPIIPGALTPTEIVTAWNFGAPSVKVFPVQSLGGANYIKSLTGPLGHIPLIPTGGVTIDNANQFLSAGAIAVGLSSELFPKALVEARNWELITQNAKNLIQNIDIT

SEQ ID NO: 80 - protein, 221 aa, Crocosphaera watsonii
MEANKTLNSSQSWRKLLTKNPAIAVIRTDNIEQGINMAKAVAAGGMNIIEITWNSHNPGEIIQQLKQDLPECTIGTGTILTLEELKEAIASGIQFCLTPHVNQTLIKTAINHDIPIIPGALSPTEIITAWQAGASCVKVFPVQAIGGIAYIKGLQGPLGSIPLIPTGGVTLDNAAYFIEAGAIAVGLSSQLFPRHALDDQKWDTITAQAQLLMERLSKFQQ

SEQ ID NO: 81 - protein, 197 aa, Aureococcus anophagefferens
MAAAIDGGFKIAEFTLTTPGCLDAVADFRGKYDGDVMVGCGTIMCTEDAEAACDAGAEFIITPVLLPEVVTWCAQRNIVCVPGCQTPTELYTAYTLGAPLQKVFPGVAGAVTWVKAVSAALPMLRLNPTSGVDLDNAGEYLSNGAASVGLVAPLFPPDAIANEDWDAIHANATKVVGNAAKAGPFVRKPTGFQLAKL

SEQ ID NO: 82 - protein, 566 aa, Bathycoccus prasinos
MLRRASSRTLRASSSLFFRAGGTGGSFPTIPIVNTATTTNKTAVMMVRCMASGPPRNATEISDELLAKLGTLSTQALIDGLWVMGWPTSHIMGARPLTEGQPKTIGRAITIQFVPQRPDIMKDKPAGMDSPEYEAFELAGPKEVIVMNSVGPWESVGGDIKFLRLMQKKVAGLVTDGSVRDTAVLRGYGFPVFCHSTTPRQGPHVHQPWACNLVINCGGVTVRPGDAIIGDQDGAVVIPAAVAQEVYDIAHSREIIEDVVKTELEQNPGPPGRYYPFHSKMIKEDSPLGKLLTSKGITPTGGFMKGMHSAARGGQEKYFGNNYYRGGTNVRSSRNNTRNSNNAMFKRNMSSYARSQSDYDEVLKTILQHKACAVLRTLHEGKVELAMDAAVRGGFKLVEFTMTTPGWADAVANFAKRTDVMMGVGTVLSVDDAKKAMDAGSRFIVSPILIPSVVEWCKENTIVCMPGCQTPTELHYAYTLGAPIQKLFPGVAGGPAWVKAVSSALPHLRINPTSGTDLDTCQDYLRNGASSVGFVAPAFDQEKIKNSDWDGIAATAKALTDAVKAA

SEQ ID NO: 83 - protein, 346 aa, Cyanidioschyzon merolae
MDGHEYSQLASKIPCFLTNMHGFLSIHDLPQQQLRFTRQRERQWRLAAAACRTCTRLTWPPRYATQRTRTATLMATFDVPTRSFPVPEKTRTYLRRQPLIVCIRLKSFILDQYAGARDALEAQRKRLLTLAAGHGVELTDQESQTAAMIIQIVETLQNAGLHNFEITMDMPGVEWALRYLHETRGTSILLGAGTVLHASQLERAIACGCRFTMSPVFLPGRSEQLIAYAREHNVLFIPGAATPSECVQASLALGEYQPVKVFPADVVGGLRFIRALAGPLSHIPLIPTSGVTFDVVEDYLSADNVYAVGVSQQILPTSRLLAGDWPQIHRLACVWRKAVESAAPSP

SEQ ID NO: 84 - protein, 273 aa, Ectocarpus siliculosus
MACVHFAACAAILCISAQSFIIDPSASSSTVHVSRCQAARLSCRETELRSKPREDALSLPISIAVLRDVQLERAVEVVRALYEEGFNMISVTADTKNFSKILRSIPRSFFPDLLLGASSVTTLDEVDVAFACAVNFVSSTSCDPAIVKRTRDLGMVSIPGVSTPAQAASAVQAGGGILKVYPATNVSPNQCRDIVNAIKTSNIPVVISGGVEPDQLQAYVKSGASGFAVGRTLFEPEKSSAEIADRARCFLLSWPQKRRRHRNVVESEDAGAS

SEQ ID NO: 85 - protein, 227 aa, Emiliania huxleyi
MAGDKYMRTPESFQAVLDRFKKDRACAVLRTPTAEKCAPAMDAAIDGGFQIVEFTLTTPGCLDRVAEYRAKYDGLKTGNVMVGCGTIMDIEDCKLALDAGAEFIVAPVLVPEVVTWCRTHNVVIIPGTQTPSEAYAAYKAGAPVQKIFPGVAGGAAWVKAVKGALPMLQLNPTSGVDLDNAGDFVQLGCSIGLVAPLFPPDVIAGNKWDVVHANATKVIGNVKAALA

SEQ ID NO: 86 - protein, 205 aa, Galdieria sulphuraria
MFQTTWVPFQISHKKCKRQRTRISFYWKQHTFKVLSQLKSEQVLDKLYKSVVVCVRQQSFSTALKTCRALMAGGLDRLEITLTTPDAFELIHTLSNEYPQATIGAGTVLKPSQVKLAAKSGALFIMSPVLVHDVIEEAKRHDLLAIPGGTTPSEIYNCFSAGASLVKLFPVESFGGLSLVKTVAGPFPDIPLLPTSGITLNALPE

SEQ ID NO: 87 - protein, 259 aa, Guillardia theta
MIALRRSSALNAVRSLAVKASAPQVRSYGERAADQSRWGMTPKTFESVLDRFAQHRACAVLRTPTAEAAPKAMQAAIDGGFKIVEFTLTTPKCLDAVADFRKKYDGKVMIGCGTIMDIEDAQNALDAGAEFIVAPVLVPEVVTWCRTRNVVCIPGCCTPTELYQAYKAGAPIQKLFPGVAGGPDFVKAVSAALPMLKINPTSGVDLNNAHEFLQKGAASVGLVAPLFDPQDISSGNWDSIKAKAEKVIKNVESVGPLKR

SEQ ID NO: 88 - protein, 545 aa, Micromonas pusilla
MSGALRSLAKRAFTAATVAPRRAANVPYLLNTARALTGPASGGHISDELLEKLGTLSTQALIDGLWVMGWPTSHVMGARPLTEGQKKMVGRAITLQFAATRPDIAADKPAGMDSPEYEAFEACAGNKGVVVMSSIGPWESVGGDIKFLRLKQLECRGLVTDGSVRDTGVLREYAFPVFSHSTTPRQGPHVHQPWAANVVINCGGVVVRPGDAVIGDDDGCIIVPASHAQQVYDIAHSREVVEEIVKEELTKNPGPPGKYYPFMTGKIKPESPLGKLLTSKGVKFTHTAARPGARATTRSVVSPMNRYKTLGARRMSTRATHPFGRPEGHYVRTHLEMEKVLKEFEIHKACAVLRTGIDGAVLPAMDAAVAGGFRLAEFTLTTPGCLDAVAEYASNRPEVLTGVGTVLTVQDAKDAMDAGAKFIVSPCLIPEVVEWCAKNNIVSAPGCGTPTEMVQAFKLGAPIQKLFPGVAGGPGWVKAVSAALPFLRINPTSGVEIDTAADYLRSGAKSLGFVAPAFPPELVKNKDWDGISEIARKIHAAVASA

SEQ ID NO: 89 - protein, 539 aa, Micromonas commoda
MYSAIRTFVRRAFPAATARQPAVTAAHVQTICMNACRGMATGPAAGGHISDELLDKLGSLSTQALIDGLWVMGWPTSHIMGARPLTEGQKKMIGRAITIQFAAARPDIAADKPSGTDSPEYEAFELVNGKEVIVMSSVGPWESVGGDIKFLRLFQRGCAGLVTDGSVRDTAVLREYGFPVFSHSTTPRQGPHVHQPWSCNTVINCGGVVVRPGDAIIGDQDGAVVIPASHAQQVYDIAHSREVIEEIVKEELTKNPGPPGKFYPFMSGKIKPNSPLGQLLTSKGVKFVHCAARPGTRLGVPTVAPTGATLGSRRTVGGPCNMSTYTQTEAHYQSVLKQFAEHKACAVLRTPIVGACTPAMNAAVDGGFKLVEFTLTTPGCLDSVAEYASRKDVLTGVGTVMTVDDAKRALDAGAQFIVSPVLIPEVVEWCAANKIVCAPGCQTPSEMVQAWKLGAPIQKLFPGVAGGPAWVRAVSAALPMLRINPTSGVEISTAADYLRAGASSLGFVAPAFPPKLVENKEWDAISKIASEIQAAVKSA

SEQ ID NO: 90 - protein, 214 aa, Ostreococcus lucimarinus CCE9901
MRTDAEFNEVLDQFAKHKACAVLRTGVEGAAYNAMDAAVQGGFKLVEFTLTTPGCLDAVSRFASRTDVLTGCGTILNIDDAKRALDAGSKFIVAPILIPEVVEWCAANKIVCIPGCQTPSELYRAYSLGAPVQKLFPGVAGGPNWVRAVASALPMLRINPTSGVEIDTAADYLRAGAASLGFVAPAFDQNAIKAGDWDAISKRATEIIAAVKAA

SEQ ID NO: 91 - protein, 553 aa, Ostreococcus tauri
MSSALRRTARTALRAARAVTTTHAGWMPKTASETRTALSAPQMAIRALATGPARHEKTISDDLLHKLGDLSTQALIDGLWVLGWPTSHVMGARPLTEGQKKLVGRAVTLRFVPQRPDIAASKPAGTESPEYEAFEQTGPNECVVMESVGPWESVGGDIKFLRLKQLGCAGLVTDGSVRDTAVLREYGFPVFSISTTPRQGPHVHQPWEANGVIVCGGIVVRPGDAVIGDQDGVVIVPASHAQEVYDIAHSREIVEGIVKDELTANPGPPGKFYPFVSGKIKKDSPLCKLLASKGYDSSKFAHTAANPGARAGIGGIPKSRNTLGSRRGMSTQQVRHKSYMRTDAEYNRVLEEFAKHKACAVLRTSVEGAAYNAMDAAVRGGFKLAEFTLTTPGCLDAVSRFADRTDVLTGCGTILNIDDAKMALDAGSKFIVAPILVPEVVEWCAANKIVCIPGCQTPSELYRAYTLGAPVQKLFPGVAGGPNWVRAVANALPMLRINPTSGVEIDTAADFLRAGASSLGFVAPAFDQNAIKAGDWDTISETARRIDAAVKAA

SEQ ID NO: 92 - protein, 254 aa, Phaeodactylum tricornutum
MAHRLALASAARLRVSHARLFSSAASSGLDRVQTVENYEHVLNTFAQVRACAVLRTATSDDCPKAMQAAIDGGFQICEFTLTTPNCLQHLADFRAKYDGKVMIGCGTILTIDDAAAAVDAGSEFIITPVMLPDVIEWCKERNIVSVPGCQTPTELVNAYRHGAPLQKLFPGVAGGPGWVKAVSSALPFLSINPTSGVDLDNAGEFLKNGAASVGLVAPLFDPVAVANKDWDQIAQNAAKVMANVREVGPYQRKK

SEQ ID NO: 93 - protein, 428 aa, Thalassiosira oceanica
MPDLNTATSAFRGKMPGRAMPTAFTERSRNEEGEYVSGVSVRLARGREIRETPPSSFAGRVTERRRGISSAERTAASLVSDDGQAKQLAGSKLHWNRGRRGSTTTLVAPSLEEVAGHRRSPRRSSSVSVRASDSALISCWEKKATTCGTSQGDRPFMSLYDLILLYPPLESSNSKNRLASSTLLRQTASRTATRQSAIRSMSTLDRTKSTDDYNRVLQTFAEIRACAVLRTPTSEACPKAMQASIDGGFKIVEFTLTTPDCLQHLSDFRAKYDGDVMVGCGTIMNTTDAENAVDAGAEFIITPVMLPDVIEWCAARNVVVVPGCQTPTEMVTAYRAGAPLQKLFPGVAGGAGWVKAVSSALPMLNINPTSGVTLDNAGEYLNNGAASVGLVAPLFDPVAIANEDWDQIAQNAAKVMANVREAGPWTRR

SEQ ID NO: 94 - protein, 157 aa, Thalassiosira pseudonana
MQACIDGGFKITEFTLTTPDCLEHLANFREKYDGDVMVGCGTIMNTEDAERAVDAGAEFIITPVMLPDVIEWCAKRNIVIVPGCQTPTELVSAYRAGAPLQKLFPGVTGGPAWVKAVSSALPMLSINPTSGVTLENAGDYLRNGAASVGLVAPLFDP

SEQ ID NO: 95 - protein, 237 aa, Physcomitrella patens
MVFGREFDRFLFYWSSVFDIRVVESCSEEVALNAGRAALNGGVSVLEVTMRTPGAAQVISKLVEEYPSATIGAGTVLTQEDAERAKEAGASFLMSPVANKDLIEAHCRSEVLFVPGVMSPSEILQARRWGASCLKLYPVSLVGGMEFVKLLKRLHPSMPIIPANGISLDVVEGYLQAGSTAVVASDAIFNKSSLARHDYAVISQQSKIAAAMGSETTRGDTSTLKYTRGGLRGRAGT

SEQ ID NO: 96 - protein, 250 aa, Selaginella moellendorffii
MAMFGERSWSVNAFGAGIDQRRSACGAQIAWPSPDDHGSCCSIHRIRESKIVACLRAHSSKLALDAARAALDGGLDVLEVTMTTPGASKVIATLVHEYPSALIGAGTVLSLDQAEEAKASGATFLLSPITVKELVEIHSFGPVLFVPGAMTPTEVYTAHSYGAHIVKIYPTDLAGGVKYVRSLRKLLAHIPMIASQGITLEMIRPYLDAGASAVVLSDAIFDSSSLSSSCFAKITQQASVAVKEAAKNAR

SEQ ID NO: 97 - protein, 236 aa, Hordeum vulgare
MLMLVAAARLPLLASQPRRRDPPRPPKALAAILRSRVIACLRAQDGETAMQAAHAAVRGGVSVLEIVMSTPGVLEVVEDLCRSYPSLTFGVGTVLNADDARKAIRAGAQFLMSPGTVMEILYDLDGSEILYIPGVLTPTEVISAYNAGANVVKVYPVSVMGGEMYMSALKKPFPLVPMVASQGITIGSIKSYMEAGASAVVLSDAIFGKELMRERDFIGISALANQATLQASQRGR

SEQ ID NO: 98 - protein, 250 aa, Amborella trichopoda
MASALCIPPSFARPTETPLRCYSHGISAKFRQFSLSSRHETLVEIQNSGVIACLRAQSGQLAMEAACAALRGGISMLEVVVSTPGVFEVITALTKDHPSSIIGAGTILNAKDATCAINAGAKFLMSPATIKEILIDLRGSSVLYIPGVMTPTEVLSAYNANAEIVKVYPVTSLGGPHYISAIKKPFSNIPMVASQGITIDTMESYIHAGASAVVLSDAIFEKEAMRRQDFDAISQLASLAASRGRAALHR

SEQ ID NO: 99 - protein, 311 aa, Amborella trichopoda
MASALCIPPSFARPTETPLRCYSHGISAKFRQFSLSSRHETLVEIQNSGVIACLRAQSGQLAMEAACAALRGGISMLEVVVSTPGVFEVITALTKDHPSSIIGAGTILNAKDATCAINAGAKFLMSPATIKEILIDLRGSSVLYIPGVMTPTEVLSAYNANAEIVKVYPVTSLGGPHYISAIKKPFSNIPMVASQGITIDTMESYIHAGASAVVLSDAIFEKEAMRRQDFDAISQLASLAASRGRAALHRLRSSTIANLYRFHENGRDVYPAMDVGELMCHGSPLFFLNMPKVTWVENLKFPPDHFENKVV

SEQ ID NO: 100 - protein, 263 aa, Beta vulgaris
MMAAGSIGLCFCPPNPKFGSSQSHPSKSSSQIKASSSCNSLSSPLIKTLSDIHTSGVIACLRAPSAELALGAARAALRGGIKVLEVVMSTPGVFEVLHQLVLENPTMTLGVGTVLNTQDARIAMKVGAKFLMSPAMIKDVLADVQDHEVLYIPGVMTPTEILSAYHAGAKLVKVYPVSALGGVEYMSALKKPFHHIPMVASQGIKLGLVADFITRGASAVVLSDAIFPKEAIARGDFDTISERAELAASTAIEAIKQLELVKQ

SEQ ID NO: 101 - protein, 236 aa, Brachypodium distachyon
MLVAAAQPPRPTSQLRRRRRDPSRPPKALASILRSRVIACLRAQDSDMALQAAHAAVRGGVSVLEIVMSTPGVLEVVEDLRRSYPSLTFGVGTVLSVDDARKAIRAGAQFLMSPGTVMEILHELEESEVLYIPGVLTPTEIISACSAGAKIVKVYPVSVMGGEMYMSALKKPFPVLPMVASQGITIDSIKSHMEAGASAVVLSDAIFNKELMGERKFVEISELANRATLQALQSGK

SEQ ID NO: 102 - protein, 265 aa, Cicer arietinum
MQLQTMASASKTLTLCQCQCQWWSSSTSSSSSLHIPSRVTCRASESHPQQLSVDKTICQIKTSGVIACLRANSAEVALEAANVAIAGGISVLEIVMSTPGVFEVLQQLVKDHPRMALGVGTVLRIEDAKTAINAGAKFLMSPANVKDIMDYVQGREVLYIPGAMTPTEILSAYDAGAKMVKIYPVSALGGFQYIEALKKPFPHVSMVASQGITIDSIVEYITRGASSVVLSDAIFDKEAIAECNFSKIYKLAQSATMLGNQAVNR

SEQ ID NO: 103 - protein, 263 aa, Cucumis melo
MLVSRSISMWSLPTIGCWNPCAFARFRVFCASSQLPVPSKDKTLRTIHNSGVIACLRASSAELAMSAACAALNGGISVLEIVMSTPGVLEVLQQLLQDYPTKTLGVGTVLNIKDAKNAVKAGAKFLMSPTMVKGIIMGDIEDEFLYIPGVMTPTEVLTAYESGCEIVKVYPVSALGGIKYISALKKPFPHISMVASQGITIESTGDYIRQGASSVVLSDAIFNKEFMDQKNFDGIFQLSKLAASQAMEALEWKQQSLLTESCH

SEQ ID NO: 104 - protein, 261 aa, Cucumis sativus
MLVSRSISMWSLPAIGCWYPCAFARFRVFCASSQLPLPPKDETLRTIHNSGVIACLRASSAELAMSAACAALNGGISVLEIVMSTPGVLEVLQQLLQDYPTKTLGVGTVLNIKDAKNAVKAGAKFLMSPTMVKGIIMGDIEGEFLYIPGVMTPTEVLTAYESGSEIVKVYPVSALGGIKYISALKKPFPHISMVASQGITIESTGDYIRQGASSVVLSDAIFNKEFMDKKNFDGIFQLSKLAASQAMEALECPLIPREQPF

SEQ ID NO: 105 - protein, 284 aa, Erythranthe guttata
MMAVANTVGLWPRNITFYSPTAAPHNLGKPFCFRSRRQNCNTNSVDVYPSKISDRVLMDIQNSGIIACLRAQSAEVAIEAARAAFRGGISVLEVTMSTPGVFEVLECLVQEYPALSIGVGTVLDRENAKVALRLGAKFLMSPAMVKGILDDVTEYQALYIPGATTPTEILSASNAGARIVKVYPVSALGGVEYMAAVKKPFPHIPMVASQGITLDSVCQYISSGAMSVVLSDAIFNKRAMFQRDFVLVNQLATIAAFTAKEAIRYKKKRVNYGRPRGKEKQGIT

SEQ ID NO: 106 - protein, 260 aa, Eucalyptus grandis
MAVLSASYCLWRRPPLILPPQPPPPPSRRYRLRACTDRSSLSPVQRTISHIENSGIIACLRANSPELAFEAACAALGAGITVLEIVRSTPGVFEVLHQLIKDHPTATLGVGTILNAEDAQNAIVAGAKFLMSPAMVKDILSDSQDCGVLYIPGVMTPTEILSAYDAGAKIVKVYPVSTLGGTHYISALKKPFPHIRMVASQGIPIDLAGEYIAKGASAVVLSDAIFTKGAMAQCNLSEISKFAHIAALQGRQAVDRRKRC

SEQ ID NO: 107 - protein, 261 aa, Fragaria vesca
MATATASLMRSPLACLSSTIVIQSSPRRPHRLRVCCSSSIQKTLTQIDSSGVIACLRANSAELAFEASCAALEGGISVLEIVMSTPGVFEVLQQLVHIYPTRVLGVGTVLNIQDAERAVIAGAKFLMSPALVKDIMDGFRCGDILYIPGVMTPTEILSAFNAGAKIVKIYPVSALGGSHYISAIKKPFPHISMVASQGITIGSVGEYIAKGASSVVLSDAIFDKEAMGQRNFNRIRQLANMAALQCKEAVDRKKNLVLNNM

SEQ ID NO: 108 - protein, 184 aa, Genlisea aurea
AQVAIEAARAAMKGGISVLEVTMSTPDVFEILQLLVEENPSASIGVGTVLNVSDAEKAVKFGAKFLMSPVMVKGILDMSMDNASSFLYIPGAMTPGEILSAHNAGAKIVKVYPVSALGGVNYIAAIKKPFSHIPLVASQGVSMETLDQYITHGASAVVLSNAIFDAEAMDRLNFCRIQQLASIA

SEQ ID NO: 109 - protein, 264 aa, Glycine max
MGTATATLSLCQWPTSSSSSSVRMHVRFSCRTPHLSPYTVDRTLCQINNSGVIACLRANSAEVAMEAAKAAIAGGISVLEIVVSTPGVFEVLQQLVKEHPTMALGVGTVLRIEDAKSAINAGAKFLMSPATVKDIMEMDYVQSGEILYIPGTMTPTEILSACDEGAKIVKIYPVSALGGFQYISALKKAFPHISMVASQGITIDSIGEYILRGASAVVLSDAIFDKDAIAQHDLDKIQKLAHSAALLGNKSVNRKSMVHLLSNL

SEQ ID NO: 110 - protein, 254 aa, Glycine max
MGTATATLSLCQWPTSSSSSSVRMHVRFSCRTPHLSPYTVDRTLCQINNSGVIACLRANSAEVAMEAAKAAIAGGISVLEIVVSTPGVFEVLQQLVKEHPTMALGVGTVLRIEDAKSAINAGAKFLMSPATVKDIMEMDYVQSGEILYIPGTMTPTEILSACDEGAKIVKIYPVSALGGFQYISALKKAFPHISMVASQGITIDSIGEYILRGASAVVLSDAIFDKDAIAQHDLDKIQKLAHSAALLGNKSVNR

SEQ ID NO: 111 - protein, 265 aa, Gossypium raimondii
MVFTCTFPCNPVFQFQRLFTRSRLQQHHSAPPLLPSNLKPSSSSSSSHHYSHTFSLIQDSAVIACLRANSAEQAMEAARAAVNGGIAVLEIVRSTPGVFEVLQALANEYPTKAFGVGTVLTAKDARTAIKAGAKFLMSPATVNDILDDVRDTDVLYMPGVMTPTEIYSAHNAGAKIVKIYPVSALGGVRYISAIRKPFSHIPMVASQGITIDSVGDYIAQGAISVVLSDAIFDKEAMSQNNFDVVNRLATSAALQGKTAVERKKR

SEQ ID NO: 112 - protein, 286 aa, Jatropha curcas
MVVAASVCSWRPNLAPQLSQNLKRFRVYSSLPPISFSSTRERTLSLIHNSGVIACLRANSAELAFEAASAALRAGISVLEIVMSTPGVFQVLQQLVKDHPTVALGVGTVLNAEDAINAKRAGAKFLMSPATVMGIMDVVLDGEVLYIPGAMTPTEILSAYDAGAKIIKVYPVSALGGTQYISALKKPFAHISMVASQGIMIDSVGDYISCGASSVVLSDAIFNKEAMAQKNFNEIYQLACLANLQGQEAVERGTTFQIEFASAEYTVFILYYLYLNRLKHWRRIFY

SEQ ID NO: 113 - protein, 259 aa, Medicago truncatula
MASASTTLTLNQCQWLSSSTSSMRLPSSRVTCKASESHPQLSVSTTVDKTLCQINNSGVIACLRANSAEVALEAANAAIAGGISVLEIVMSTPGVFEVLQQLVKEHPRITLGVGTVLRIEDAKTAIKAGAKFLMSPANVKDILNYVQGGDILYIPGTMTPTEILSAYDAGAKMVKIYPVSALGGFQYIAALKKPFPHVSMVASQGITIDSMEEYIMRGASAVVLSDAIFDKEAISQCNFSKIYKLAQSATLLGNQAVNR

SEQ ID NO: 114 - protein, 254 aa, Musa acuminata
MALASCFSPPFRPPLCAGGRSLSPVACACAQTVTTAPAAPSLPKALPAILDSRLIACLRARDGNTAMEAACAALSGGISVLEIVMSTPGVLEVIRGLLKDYPSSVIGVGTVLNAEDARKAVKAGAQFLMSPGTVMEILLDLQNTDVLYIPGAMTPTEVLSAYNAGARIIKIYPVSVLGGCDYIKALKKPFPHIPMVASQGTTTDSIRKYIECGASAVVLSDAIFEKEAMRHRNFDEIHRLAHLATLQVGQAGKC

SEQ ID NO: 115 - protein, 263 aa, Nelumbo nucifera
MATASFSLSPSPFSRSDFRPSSTQARLRIPCRSDGTSLSPLSRTLSAIESSGVIACLRASSGDLAREAAQAALSGGISVLEIVMSTPGVFEVLEGLVQDHPTSIIGVGTVLNALDARKAIKAGARFLMSPVTVKDILEDIQGGEILYIPGVMTPTELWAAYNSGARVVKVYPISALGGTQYISSLKKPFPHIPMVASQGITLDSIGTYIAGGASAVVLSDAIFDKEAMCQQNFDTIYQLAQLAALQGHEAVQRKKRCMLSLAS

SEQ ID NO: 116 - protein, 264 aa, Nelumbo nucifera
MATASFSLSPSPFSRSDFRPSSTQARLRIPCRSDGTSLSPLSRTLSAIESSGVIACLRASSGDLAREAAQAALSGGISVLEIVMSTPGVFEVLEGLVQDHPTSIIGVGTVLNALDARKAIKAGARFLMSPVTVKQDILEDIQGGEILYIPGVMTPTELWAAYNSGARVVKVYPISALGGTQYISSLKKPFPHIPMVASQGITLDSIGTYIAGGASAVVLSDAIFDKEAMCQQNFDTIYQLAQLAALQGHEAVQRKKRCMLSLAS

SEQ ID NO: 117 - protein, 287 aa, Nicotiana sylvestris
MAIASFSLPLTYFPQRPFSYCCSSSSLKQSNSAPSNCIANKALQEIQNSGVIACLRAQNADLATRAARAALDGGISVLEIVVSTPDAFEVLRNLVHDYPAKTFGVGTVLQAKDAKDSIKSGAKFLMSPATVMDILVGVSVGNALYIPGVMTPTEILSAFNAGAKIVKVYPVSALGGVGYISALKRPFPHIPMVASQGVTLDLIGQYIGQGATAVVLSDAIFDKEAMIQRNFDKIYQLASVAALQGKQAVERKDSCAGFGMESFTPLSGPLGESSLVESRLVGPMDFG

SEQ ID NO: 118 - protein, 260 aa, Nicotiana tomentosiformis
MAVASFSLPLTYFPQRHKLPFSCCCSSSVKQSNSVPSNCIANKALQEIQNSGVIACLRTQNADLATRAARAALDGGISVLEIVVSTPDAFEVLRNLVHHYPTKTFGAGTVLQAKDAKDSIKSGAKFLMSPATVMDILVGVSERDALYIPGVMTPTEILSAFNAGAKIVKVYPVSALGGVGYISALKRPFSHIPMVASQGVTIDLVGQYIGQGASAVVLSDAIFDKEAMSQWNFDKIYQLASHAALQGKQAVESQGQNISC

SEQ ID NO: 119 - protein, 190 aa, Oryza brachyantha
MKAAHAAVRGGVSVLEVVMSTPGALEVICDLRRSYPSLTFGIGTVLNPEDARKAIRAGAQFLMSPGTVMEILHDLEEREVLYIPGVLTPTEVISASSAGAEVVKVYPVSVMGGEAYMRALKKPFPSLPMVASQGISIDSINGYLEAGASAVVLSDAIFDKELMRERKFDEISELANLSILQASQSRMTKI

SEQ ID NO: 120 - protein, 244 aa, Oryza sativa
MLVAAAAHSPASTALPRRRRRAAAPPSRKTPESQALRSILHSRVIACLRAQDGETAMQAARAAVRGGVSVLEIVMSTPGALEVVIGDLRRSYPSLTFGVGTVLNPEDARKAITAGAQFLMSPGTVMEILHALKESEVLCIPGVLTPTEVISASNAGAEVVKVYPVSVMGGEVYMLALKKPFPFLPMVASQGISIDSIKGYLEAGASAVVLSDAIFDKELMRERKFDEISELANLATLRASQSGT

SEQ ID NO: 121 - protein, 187 aa, Oryza sativa
MQAARAAVRGGVSVLEIVMSTPGALEVIGDLRRSYPSLTFGVGTVLNPEDARKAITAGAQFLMSPGTVMEILHALKESEVLCIPGVLTPTEVISASNAGAEVVKVYPVSVMGGEVYMLALKKPFPFLPMVASQGISIDSIKGYLEAGASAVVLSDAIFDKELMRERKFDEISELANLATLRASQSGT

SEQ ID NO: 122 - protein, 255 aa, Phaseolus vulgaris
MDAATATLSMCPCHIFSSSLPSLRMHVRVSSRIPHLSASAVDKTLSQINNSGIIACLRANSAEVAMKAANAAIAGGVSVLEIVVSTPGVFEVLQQLVKEHPTMALGVGTVLRIEDAKNAIDAGAKFLLSPATVKDIMVMDYVQSGKVLYIPGTMTPTEILSACDAGAKMVKIYPVSALGGFQYISALKKTFPHVSMVASQGITIDAIGEYILRGASSVVLSDAIFDKEAIEQLNFDKIQKLAHSATLLGNKAVNR

SEQ ID NO: 123 - protein, 256 aa, Phoenix dactylifera
MALAGFSRFHRPPPPPPLHLRRPSASARASAPSVSPLPRALIEIERSRVIACLRASDGKLAMEAACAALSGGISVLEIVMSTPGVLEVIKGLQKDHPSSVIGVGTVLNAEDARKAAKAGAQFLMSPCTVMEILVDIQDNEVLYIPGVMTPTEVLFAYNAGARIVKVYPVSMLGGYKYISALKKPFPHIPMVASQGIATDSIEGYIGGGASAVVLSDAIFEKEAMRQRNFDEIQRLAYLATLLADRARRPRGQGYIA

SEQ ID NO: 124 - protein, 261 aa, Populus euphratica
MDMVAASWRLTPQSLPLLSPKVKSFRVYSSSSSSSLSLSPIIQKTSSLIQNSGVIACLRANSAELAYEAATAALNGGISVLEIVMSTPGVFQVLRQLVKDYPTLALGVGTALNAEDARNAMNAGAKFFMSPATVKDIMDDVVKDEILYIPGVMTPTEILSAYDAGAKMVKVYPVSALGGVQYISALKKPFPHIPMVASQGIKIDSIGEYISSGASSVVLSDAIFDKGAMTQRNFNAIHQLASLAALEGKEAIERKRSCTTS

SEQ ID NO: 125 - protein, 259 aa, Populus trichocarpa
MATSWRLTPQSLPLLSPKVKSFRVYSSSSSSSLSLSPIIQKTSSLIQNSGVIACLRANSAELAYEAATAALNGGISVLEIVMSTPGVFQVVLRQLVKDYPTLALGVGTALNAEDARNAMNAGSKFFMSPATVKDIMDDVVKDEILYIPGVMTPTEILSAYDAGAKMVKVYPVSALGGVQYISALKKPFPHIPMVASQGITIDSIGEYISSGASSVVLSDAIFDKGAMTQRNFNVIHQLASLAALEGKEAVERKRSCTTN

SEQ ID NO: 126 - protein, 154 aa, Prunus persica
VGTVLNVEDAKRAMIAGAKFLMSPAMVKDILDDFQYSGVLYIPGVMTPTEILSAYDAGAKIVKVYPVSALGGSQYISAIKKPFPHISMVASQGITTDSVGDYIAQGASSVVLSDAIFDKEAMGQKNFTKIYQLANAAALHGNEAVDRGQQYTSN

SEQ ID NO: 127 - protein, 304 aa, Sesamum indicum
MMAVASSGGSIWPLNLFFSSSKIKPFLTKASVHNYSKRFSSHVRCKNSYKNSVAVYPGTVADRVLMDIQNSGIIACLRAQSAEMAIEAAQAALSGGISVLEITMSTPGVFEALQFLVHEYPSASLGVGTVLDREDAKDALKCGAKFLMSPAMVKEILDDVAEGQALYIPGAMTPTEILLASTAGAKIVKVSCFFLLIQSLVYPVSALGGVQYIAAVKKPFPNIKMIASQGITIGQMVQRFPAYATIFTSTSAMISPSFLQVEGNFIIRIPNVILPFAKLCPHLNAHVKEYLERNRQIEDMMLTI

SEQ ID NO: 128 - protein, 245 aa, Setaria italica
MRVAATQLPLPTASPPRRRRRAQHMPTAAAPRPPPRALAAILRSRVIACLRAEDGETALQAAHAAVLGGVTVLEVVMSTPGVLEVIEDLCRSYPSLTFGVGTVLNAADARKAIGAGAQFLMSPGTVMEVLHDLEESKVLYIPGVLTPTEVLSACSAGAKVVKVYPVSVMGGEVYMSALKKPFPLVPMVASQGIQIGSIKGYVEAGASAVVLSDAIFDKELMRKGKFSEISELASQATFEALQSTK

SEQ ID NO: 129 - protein, 239 aa, Solanum lycopersicum
MRCSSSSSSSFENPIFVHSSNGIANKALQEIRNSGVIACLRAQNADLANRAARAALDGGISALEIVVSTPDVFEVIRDLVHDYPAKTFGVGTVLQAKDAKDSIKAGAKFLMSPATVMDILVGVSESDILYIPGVMTPTEILSAFSAGAKIVKVYPVSALGGVGYISALKKPFSHIPMVASQGITIDLIGQYIGQGASAVVLSDAIFDKEAMSQQNFDKIYQLASHSALQGKQAVERLEC

SEQ ID NO: 130 - protein, 256 aa, Solanum tuberosum
MAIASFSLPFTYLQKPSSCSCRCSSSSFEKPIFAHSSNGIANKALQEIRNSGVIACLRAQNADLANRAARAALDGGISALEIVVSTPDVFEVLRDLVHDYPAKTFGVGTVLQAKDAKDSIKSGAKFLMSPATVMDILVGVSESDVLYIPGVMTPTEILSAFSAGAKIVKVYPVSALGGVGYISALKRPFSHIPMVASQGITIDLIGQYIGQGASAVVLSDAIFDKEAMSQQNFDKIYQLASHAALQGKQAVERSEC

SEQ ID NO: 131 - protein, 245 aa, Sorghum bicolor
MRVAASQLPLPTTAAPPRRRRRAPAPPTAAPRPPPRALAAILRSRVIACLRAQDGETALQAAHAAVRGGITVLEVVMTTPGVLEVIEDLCRSYPSLTFGVGTVLNAADARKAIEAGAQFLMSPGTVLDILHDLEESNVLYIPGVLTPTEVLSACSAGAKVVKVYPVSVMGGEAYMSALKKPFPVVPMVASQGIKIGSIKGYMEAGASAVVLSDTIFDNQLMKDRKFSKISELANLATLEALQFTK

SEQ ID NO: 132 - protein, 256 aa, Spinacia oleracea
MAGRSIGLCFCPPMVGNSPCLSQLKASSSCNSPPQTSTLSPPLIKTLSDIHSSGIIACLRAPSAEIAVGAARAALRGGIKVLEVVMSTPGVFEVLQQLVLEYPTMTLGVGTVLNAEDAKVAMKVGAKFIMSPAMIKDVLADVHDHEVLYIPGVMTPTEILSAYNAGARIVKVYPVSALGGVEYISAVKKPFHHIPMVASQGIKMDLVEEYITRGASAVVLSDAIFCKEAIARGDFDTISEQAQLAASIGIEAKKQL

SEQ ID NO: 133 - protein, 273 aa, Theobroma cacao
MVFTCTLTCLSGTQSQRLFTWNSVQQHYSALPLPPYPFKPCCSSSSRNHNHTFSLIQDCAVIACLRANSAEQAMEAARAAVNGGIEVLEIVRSTPGVFEVLQALVKEYPTKAFGVGTVLNAEDAKTAMNAGAKFLMSPATVKDIMDDVQDADVLYMPGVMTPTEIFSAYNSGAKIVKIYPVSALGGTRYISAIMKPFPHIPMVASQGITIDSVGDYIAQGAISVVLSDAIFDKEAMGRNNFNAIHQLAKSAALQGKIAVEWKRNCRLTFFPHS

SEQ ID NO: 134 - protein, 235 aa, Triticum urartu
MLVVAAAKLPLLASQSPRRDPPRSPKALAAILRSRVIACLRAQDGETAMQAVHAAVRGGVSVLEIVMSTPGVLEAVEDLRRIYPSLTFGVGTVLNADDARKAIRAGAQFLMSPGTVMEILHDLDGSEVLYIPGVLTPTEVIYAFNAGAKVVKVYPASVMGGEMYMSALKKPFPLVPMVASQGITIGSIKSYMDAGASAVVLSDAIFDKELMRERNFIGIAALANQATLQASQCGR

SEQ ID NO: 135 - protein, 244 aa, Zea mays
MRVAASQLPLPTTTPARRRRRASAPPTAAPRPPPRALAAILRSRVIACLRAQDSETALQAAHAAVHGGITVLEVVMTTPGVLEVIEDLCRSYPSLTFGVGTVLNAADARKAICAGAQFLMSPGTVLDILHDLKESNVLYIPGVLTSTEVLSACSAGAKVVKVYPVSVMGGEMYMSALKKPFPLVPMVASQGIKIGSIKGYMEAGASAVVLSDAIFDSQLMRDGKFNEISELANLATLEALQFTK

SEQ ID NO: 136 - protein, 561 aa, Synechocystis PCC6803
MSNNPRSQVITQGTQRSPNRAMLRAVGFGDDDFTKPIVGIANGYSTITPCNMGINDLALRAEAGLRTAGAMPQLFGTITISDGISMGTEGMKYSLVSREVIADSIETVCNGQRMDGVLAIGGCDKNMPGAMIAMARLNIPSIFVYGGTIKPGHYAGEDLTVVSAFEAVGQYSAGKIDEETLYGIERNACPGAGSCGGMFTANTMSSAFEAMGMSLPYSSTMAAVDGEKADSTEESAKVLVEAIKKQILPSQILTRKAFENAIAVIMAVGGSTNAVLHLLAIANTIGVPLSLDDFETIRHKVPVLCDLKPSGKYVTTNLHAAGGIPQVMKILLVNGILHGDALTITGQTIAEVLADIPDQPPAGQDVIHSWDDPVYQEGHLAVLKGNLATEGSVAKISGVKKPVITGPAKVFESEEDCLEAILAGKIQAGDVVVVRYEGPKGGPGMREMLAPTSAIIGAGLGDSVGLITDGRFSGGTYGLVVGHVAPEAYVGGAIALVQEGDQITIDAGKRLLQLNISEEELAQRRAQWTPPQPRYPRGILAKYAKLVSSSSLGAVTDIDLF

SEQ ID NO: 137 - protein, 206 aa, Synechocystis PCC6803
MLRQHRAIAVIRTDSIDLSLQLAKVAIEAGMGLVEITWNSDQPGETIAHLRHQFPHCAIGTGTVLSQEDLLQAVASGAQFCFTPHSEPELITSAVAHGIPIIPGAFSPTEIVQAWQRGATAVKVFPIKTLGGVDYIQALQGPLGQIPLIPTGGVTLANAQAFLAAGAIAVGLSGQLFPPNLVAIKAWREIGDLIRTLLKEIQTPRG

SEQ ID NO: 138 - protein, 555 aa, Phaeodactylum tricornutum
LTQTKVQGASQAMLYATGITEEDLDKPQVGICSVWYEGNPCNMHLLDLSEKVKKGVEDASCVGYRFNTVGVSDGISMGTSGMRYSLQSRDLIADSMETTMGGQWYDGLIALPGCDKNMPGCIMAMGRLNRPGIMVYGGTIRAGKQPSTGNSLDIVSAFQSYGEYVYDKITEEERKEILQHACPGQGACGGMYTANTMATAIEALGMSLPYSSSSPADSKEKADECYRSGEAMYRLLELDLKPRDIMTKAAFENAMRMVMVTGGSTNAVLHLIAMSRSVQNPEVAITLEDFQRISNQTPFLADLKPSGKYVMEDVQNIGGTPGLIKFMIDNGLFDGSQMTVSGKTHAENLKDHPGLTPGQDIIRPLSDPVKKTGHLMMMYGNLCPGGGVAKITGKEGETFTGTARVYDNEQLMMRGLENKEIKAGDVVIIRYEGPKGGPGLPEMLTPTSAIMGAGLGDKVALLTDGRFSGGSHGFCIGHITPEAQVGGPIALVKNGDPIRIDARAEQRTIDLLISDEEWEKRRTEWTPPPLRATQGTLFKYIQCVATASEGCVTDE

SEQ ID NO: 139 - protein, 595 aa, Hordeum vulgare
MQSLALTPPSLPASGPTPASGRRLATRVRAASAVSDEPKLNKYSARITEPKSQGASQAVLYGVGLTDADLRKPQVGVSSVWYEGNTCNMHLLQLAEAVRDGIRDAGMVAFRFNTVGVSDAISMGTRGMCYSLQSRDLIADSIETVMGAQHYDANISIPGCDKNMPGTIMAMGRLNRPSIMVYGGTIKPGHFQGNSYDIVSAFQSYGEFVSGSISDEERKNVLRNSCPGAGACGGMYTANTMASAIETMGMSLPYSSSTPAEDSLKLDECRLAGKYLLELLKMDLKPRDIITEKSLRNAMVIIMALGGSTNAVLHLIAIARSVGLQLTLDDFQKVSDQVPFLADLKPSGKYVMEDLHKIGGTPAVIHYLLEQGLLDGDCMTVTGKTLAENAKIFPPLSEGQQIIRRLDNPIKSTGHIQILYGNLAPEGSVAKITGKEGLFFSGPALVFEGEESMITAISENPANFKGKVVVIRGEGPKGGPGMPEMLTPTSAIMGAGLGKECALLTDGRFSGGSHGFVVGHVCPEAQEGGPIGLVEDGDTITIDVVKKVIDVDLTEDQLEQRRRKWSPPPHKVTSGALWKYIKLVSSASSGCVTDE

SEQ ID NO: 140 - protein, 624 aa, Guillardia theta
MLLVAAVLSAMSAGSLAFMPGHTGNAFVSQASQTSSSFSSPSVLPRALGARGRARVSAAGLSMQINKYSRTLTQKKSQGASQAMLYATGLKPEDMNKAQVGISSVWYEGNPCNMHLLDLAGEVKKGVEAAGMVGYRFNTIGVSDGISMGTDGMSYSLQSRDIIADSIETVVCGQWYDGNIALPGCDKNMPGTIIAMARHNRPSIMIYGGTIRAGSAPEIDRPVLDIVSAFQSYGEFQYGKITEEQRQTIVQKSCPGPGACGGMYTANTMATSIEALGMSLPYSSSYPADSKEKKMECYNSGKAMMKLLEMDLKPRDIMTREAFENAMVMVMATGGSTNAVLHLIAMARSVGIKLTVDDFQRVSDRVPMLADMKPSGKYVMEDIQNIGGTPAVMKYLVDKNLFDDSQMTVTGKTIRENLESLPGLTDGQKIILPVEAPIKKTGHLQILYGNVAPEGSVAKITGKEGETFSGTAAVFDSEEDMLAALDRKEIKAGMVVVIRYEGPKGGPGMPEMLTPTSAIMGAGLGNDVALITDGRFSGGTHGFCIGHVTPEAQVGGPIALIRNGDPIQIDARSSVRTLNVLVSDEEMAKRKAEWKAPPLKATQGTLYKYIKAVSTASEGCVTDA

SEQ ID NO: 141 - protein, 503 aa, Acetobacter aceti
MDAAGSSSKIQADGKRADSEAVAFDFVIVGATGDLTMRKLLPAFYECFRRGQIDRTTKIIGVARSDLSIQDYRVKAEEALRDFVAADSYDDVTVRDFLGLVHYVSLDMSDRATDWSGLISLLSTNGGRPRVFYVATAPRLYVPTADAIGHNKLITESTRIVLEKPIGTDQATAAEINDGVGRYFTEEQIFRIDHYLGKQTVQNILALRFANPILERVWNTDSIAHVQITAAETVGVGKRGPYYDNAGALRDMVQNHLLQVLSLVAMEPPTAFSAMDLRDEKLKILRALKPMSDHDIATDTVRAQYGEGHPNGKLAPGYLEDLGAPTSTTETYLAIRAEIRTARWAGVPFYIRTGKRMASKETTVVVEFRPQPWAIFADNPEPSQLVLRIQPNEGVSLSLASKDPASEQYRLKEAVLDVDYVKAFNTRYPDSYEDLLMAAVRGDQVLFIRRDEVEASWRWIEPILRGWEKNIRPLETYPAGTQGPASSDQLLARDGFVWKENIQ

SEQ ID NO: 142 - protein, 491 aa, Hafnia alvei
MAVTSTAQACDLVIFGAKGDLARRKLLPSLYQLEKAGHIHPDTRIIGVGRADWDKDAYTKVVREALDTFLKEKVDEELWTTLSARLDFCNLDVNDTQHFKNLAKMLDQKKRVTINYFAMPPSTFGAICQGLGASGLNKDPSRIVMEKPLGTDLESSRAINNQVAEFFDESQVYRIDHYLGKETVLNLLALRFANSLFANNWDNSMIDSVQITVAEEVGIEGRWGYFDKAGQMRDMIQNHLLQILTMIAMSPPADLSTDRIRDEKVKVLRSLRRINHSNVRDTTVRGQYTAGFVQGKKVPGYLEEEGANKTSNTETFVSIRVDIDNWRWAGVPFYLRTGKRLPSKCSEVVIYFKNPPLNLFSDSYQELPQNKLTIRLQPDEGVQIDILNKVPGLEHKHRLQTTKLDLSFSDTFHQEHLADAYERLLLETMRGIQALFVRRDEVEEAWKWVDSIMEAWAADNESPKPYQAGTWGPVASVAMISRDGRAWNEFE

SEQ ID NO: 143 - protein, 491 aa, Proteus vulgaris
MAAISTAQACDLVIFGTKGDLARRKLIPSLYQLEKAGYIHPDSRIIGVGRADWDVEAYKNVAHEALKTFLKEEINPEIWQRLSDRLDFCNLDVNETDHFIELSKHLKQDKLPAIYYFAMPPSTFSAMCQGLGHAKLNKEPNRVVMEKPLGTDLASSVSINDSVAKYFKETQIYRIDHYLGKETVLNLLALRFANSLFVNNWDNKTIDHVQITVAEEVGIEGRWGYFDQAGQMRDMVQNHLLQILTMIAMSPPADLTADSIRQEKVKVLRSLRRIDNTNIREKTVRGQYTGGFVQGKKVPGYLDEEGANKTSNTETFVAIRADIDNWRWAGVPFYLRTGKRLPSKCSEVVVYFKKPALNIFSETYQELPQNKLTIRLQPDEGIDIEVLNKAPGLDHKHRLQTTKLDLSFSETFNQTHLADAYERLLLEAMRGIQALFVRRDEVEEAWKWVDSIINAWECDNELPKPYQAGTWGPVASVAMITRDGRSWNEIE

SEQ ID NO: 144 - protein, 507 aa, Pseudomonas fluorescens
MTHTIRRKSKAEPAPPTTLFLFGAHGDLVKRLLMPALYNLSRDGLLDDGLRIVGVDHNAITDEAFAQKLEDFIRTEVAAKVGKGDQMLDPALWAKLAKGISYVQGDFLDDSTYSALAAKIADSGTGNAVFYLATAPRFFSEVVRRLGSAGLLEETAEAFRRVVIEKPFGSDLQTAEALNACLLKVMSEKQIYRIDHYLGKETVQNILVSRFSNSLFEAFWNNHYIDHVQITAAETVGVETRGSFYEHTGALRDMVPNHLFQLLAMVAMEPPAAFGADAVRGEKAKVVGAIRPWTTEEARENSVRGQYSAGEVAGKPVSGYRDENNVSPDSTTETYVALKVMIDNWRWVGVPFYLRTGKRMSVRDTEIVICFKPAPYAQFRDTEVDELQPTYLRIQIQPNEGMWFDLLAKRPGPALNMANIELGFAYKDFFEMQPSTGYETLIYDCLTGDQTLFQRADNIENGWRAVQPFLDAWQQDASVQTYAAGEDGPKAADDLLTRDGRVWHGLG

SEQ ID NO: 145 - protein, 485 aa, Zymomonas mobilis
MTNTVSTMILFGSTGDLSQRMLLPSLYGLDADGLLADDLRIVCTSRSEYDTDGFRDFAEKALDRFVASDRLNDDAKAKFLNKLFYATVDITDPTQFGKLADLCGPVEKGIAIYLSTAPSLFEGAIAGLKQAGLAGPTSRLALEKPLGQDLASSDHINDAVLKVFSEKQVYRIDHYLGKETVQNLLTLRFGNALFEPLWNSKGIDHVQISVAETVGLEGRIGYFDGSGSLRDMVQSHILQLVALVAMEPPAHMEANAVRDEKVKVFRALRPINNDTVFTHTVTGQYGAGVSGGKEVAGYIDELGQPSDTETFVAIKAHVDNWRWQGVPFYIRTGKRLPARRSEIVVQFKPVPHSIFSSSGGILQPNKLRIVLQPDETIQISMMVKEPGLDRNGAHMREVWLDLSLTDVFKDRKRRIAYERLMLDLIEGDATLFVRRDEVEAQWVWIDGIREGWKANSMKPKTYVSGTWGPSTAIALAERDGVTWYD

SEQ ID NO: 146 - protein, 251 aa, Acetobacter aceti
MSTQDVMTAREVVLEDPDAVAQWTADFLLEKALSARGVFRIALSGGSTPRKLYALMAEAGYADRFPWSRTEFFYGDDRFVPAESADSNRGMVQSILFDHVPVPTKNIHPMPDRGEPDQAARSYEADLQQAYGGNTLIANKPLFDVVLLGLGENGHTASLFPRQPVLEEKIRWVASCVPDDAPHTRLTLTYPAIASSRHVLFLVTGTSKAGVLGRVRAGDAAEPASHICSDGEVVWIIDRGAAGQAEGKTER

SEQ ID NO: 147 - protein, 231 aa, Hafnia alvei
MAQFKEFPSAQALNEQFAEEIAAALSHSISVQGEASLVVSGGRTPLGLFEALTQQAIEWSKVSITLADERWVEANDDSSNEKLVREHLLKGHAATAKFVGLKNSHATPFAGAADTERALQSIARPFDVVILGMGDDGHTASLFPGAENLFPALAMDSGRVCMGMTPLTAPLDRITLTLPALLNSRHIYLHVVGGSKRDVYQRAIQGTDVNEMPIRAVLHQTQTPVDVYWTA

SEQ ID NO: 148 - protein, 329 aa, Proteus vulgaris
MKQVVYIASPESQQIHVYALNTEGEMDLLQVVEVAGQVQPMIASADGKYLYVGIRPHFSVVTFAIADDGQLEQKAITSIPSTPVHLCLDKTERFLFVPSYHQGNLAVLPINEKGIPQSAIQIIEGLNKPHSSNIDWNNKQLIVPCLGEDHIRLFNLSEKGYLTEAGTEELTTAQNAGPRHMAFHPNGDVFYCLNELDATINVYRRMGDLYRLMQTVDIVPETFNSTRWASDIHITPDGRFLYASERSESFICAMAISESGDYLQPVGYYPTQTQPRGFNLDNIGNYLISCGQKSDFASVSRIDKHTGALTELAYYPVGKGAMWVTVVTK

SEQ ID NO: 149 - protein, 232 aa, Pseudomonas fluorescens
MKLPEGVNAHEFKSPVLLAEGLALNVAKQLSEALAARGNAVLVVSGGRSPVAFFQHLAKQELDWSKVVVTLADERWVPVEHADSNAGLLKQYLLKGPAAKAQFLSLYSAAANVEQAAEQADRLLAELPPIDVLVLGMGDDGHTASLFPDSPNLVEALQADGTRRCWPMLAPSVPRQRLTMSRALLASARHKILSISGQSKLTTLNAALASNDVAAMPVRAFLQPTLEIYWCP

SEQ ID NO: 150 - protein, 235 aa, Zymomonas mobilis
MTEAEWWEFENVEAMAKQIADDIEFIIKQAIEKKGRALIIVPGGSTPKLVFPTLAARDLDWSKVTLMLTDDRLVAKDNPLSNFGLLTKHFGSSGAELVSLIDENYLDDRAAAGRAADQKLASYKWPADLVWLGMGNDGHTASIFPGPNFDEAVNGPRERRALGLLPVPLPPEAPVARVTLSLSTLASAHTVMVVITGDHKRTVLTDALKEGASSRLPVGRVLGETEASIDVYWSK

SEQ ID NO: 151 - DNA, 1470 bp, Arabidopsis thaliana
gaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatc

SEQ ID NO: 152 - DNA, 15551 bp, Artificial Sequence
  Synthetic construct plasmid pED01
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggatccaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgactgatctgcattcaacggtagaaaaggttaccgcgcgcgttattgaacgctcgcgggaaacccgtaaggcttatctggatttgatccagtatgagcgggaaaaaggcgtagaccgtccaaacctgtcctgtagtaaccttgctcatggctttgcggctatgaatggtgacaagccagctttgcgcgacttcaaccgcatgaatatcggcgtcgtgacttcctacaacgatatgttgtcggctcatgaaccatattatcgctatccggagcagatgaaagtatttgctcgcgaagttggcgcaacggttcaggtcgccggtggcgtgcctgctatgtgcgatggtgtgacccaaggtcagccgggcatggaagaatccctgtttagccgcgatgttatcgctttggctaccagcgtttctttgtctcatggtatgtttgaaggggctgcccttctcggtatctgtgacaagattgtccctggtctgttgatgggcgctctgcgctttggtcacctgccgaccattctggtcccatcaggcccgatgacgactggtatcccgaacaaagaaaaaatccgtatccgtcagctctatgctcagggtaaaatcggccagaaagaacttctggatatggaagcggcttgctaccatgctgaaggtacctgcaccttctatggtacggcaaacaccaaccagatggttatggaagtcctcggtcttcatatgccaggttcggcatttgttaccccgggtaccccgctccgccaggctctgacccgtgctgctgtgcatcgcgttgctgaattgggttggaagggcgacgattatcgtccgcttggtaaaatcattgacgaaaaatcaatcgtcaatgctattgttggtctgttggcaaccggtggttccaccaaccataccatgcatattccggccattgctcgtgctgctggtgttatcgttaactggaatgacttccatgatctttctgaagttgttccgttgattgcccgcatttacccgaatggcccgcgcgacatcaatgaattccagaatgcaggcggcatggcttatgtcatcaaagaactgctttctgctaatctgttgaaccgtgatgtcacgaccattgccaagggcggtatcgaagaatacgccaaggctccggcattaaatgatgctggcgaattggtctggaagccagctggcgaacctggtgatgacaccattctgcgtccggtttctaatcctttcgcaaaagatggcggtctgcgtctcttggaaggtaaccttggccgtgcaatgtacaaggccagtgcggttgatcctaaattctggaccattgaagcaccggttcgcgtcttctctgaccaagacgatgttcagaaagccttcaaggctggcgaattgaacaaagacgttatcgttgttgttcgtttccagggcccgcgcgcaaacggtatgcctgaattgcataagctgaccccggctttgggtgttctgcaggataatggctacaaagttgctttggtaactgatggtcgtatgtccggtgctaccggtaaagttccggttgctttgcatgtcagcccagaagctcttggcggtggtgccatcggtaaattacgtgatggcgatatcgtccgtatctcggttgaagaaggcaaacttgaagctttggttccagctgatgagtggaatgctcgtccgcatgctgaaaaaccggctttccgtccgggaaccggacgcgaattgtttgatatcttccgtcagaatgctgctaaagctgaagacggtgcagtcgcaatatatgcaggtgccggtatctaacctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaattaattaatacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcagatctaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgcgtgatatcgattccgtaatgcgtttggcaccggttatgccggtcctcgtcattgaagatattgctgatgcaaaacctatcgcagaagctttggttgctggtggtctgaacgttcttgaagtaacgcttcgcaccccttgtgctcttgaagccatcaagatcatgaaagaagttccgggtgccgttgttggtgccggtacggttctgaacgcaaaaatgctcgaccaagctcaggaagctggttgcgaatttttcgttagcccgggtctgaccgctgacctcggcaagcatgctgttgcccagaaagcagctttgcttccaggtgttgctaatgctgctgatgtgatgcttggtcttgaccttggtcttgatcgcttcaaattcttcccggctgaaaatatcggtggtttacctgccctgaagtccatggcttctgttttccgtcaggttcgtttctgcccgaccggcggtatcaccccgacgtcagctcctaaatatcttgaaaacccgtccattctttgcgtcggtggtagctgggttgttccggctggcaaaccagatgtcgcaaaaatcacggcactcgctaaagaagcttctgctttcaagcgcgctgctgttgcctaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 153 - DNA, 15407 bp, Artificial Sequence
  Synthetic construct plasmid pED02
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggatccaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgtccaacaatcccagaagccaggtaattacccagggcacccaacggagtcccaaccgggccatgctccgggccgttggttttggcgatgatgactttacgaaacccattgtgggtattgccaacgggtacagcaccattaccccctgcaacatgggcattaatgatctggctctacgggccgaggccggcttacggacagcgggggccatgccccaattgtttggcactatcaccatcagtgacggtatttccatgggtacggaagggatgaaatattccctagtttcccgggaagtgattgctgattccatcgaaacagtatgcaatggccaacgcatggacggagttttggcgatcgggggctgtgataagaatatgccgggggcaatgattgccatggctcgcctcaatattccctctatctttgtttacggtggcactattaaacccggacattatgccggggaagatttaacagtggtcagtgcctttgaagcggtgggccagtacagcgctggcaaaattgatgaggaaaccctctacggcattgaacggaatgcctgtcctggggctggttcctgtggggggatgttcaccgccaacaccatgtcctccgcctttgaagctatggggatgagcttgccctattcttctaccatggccgcagtggatggggaaaaagccgacagcaccgaggagtccgccaaggttttggtggaagccatcaaaaaacagattttgcccagtcaaattctgacccggaaagcttttgaaaatgccattgccgtgattatggccgtggggggctccactaatgcagtgctccatctgctggcgatcgccaacaccattggggtgcccctaagcttggacgattttgaaaccattcgccacaaagtacctgtactgtgcgacctcaaaccttccggcaaatacgtcaccactaatctccacgccgccggtggcattccccaggtgatgaaaatattgctggtcaatggcattctccacggtgacgctttaacgattacaggacagaccattgctgaagttttagccgatattcctgaccaaccccctgccggccaggacgtaatccattcctgggatgaccctgtttatcaagaaggtcacctggcagtgctcaaaggtaacctggccaccgaaggcagtgtggctaaaatcagcggtgttaaaaagccggtgatcactggtcccgccaaagtatttgaatctgaagaagattgcctagaagcgattctagccggcaaaattcaagcaggagatgtggtggtcgtgcgctatgaagggcccaaaggtggccccggtatgcgggaaatgctagcccccacttctgccatcattggggcagggttgggagactccgttggcctaatcaccgatggccgcttttctggtggtacctatggcttagtggtgggtcacgttgcccccgaagcttatgtgggaggggcgatcgccttggtgcaagagggggatcaaatcaccatcgatgctggcaagcggttactgcaactcaacatcagtgaagaggaattggcccaacggcgggcccaatggactcctcctcagccccgctatccccgggggattttagccaagtatgccaagttagtttcctccagcagccttggtgcagtgactgatatagacttgttctaacctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaattaattaatacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcagatctaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgttgcgacagcaccgggcgatcgccgttattcgcactgattccatcgacctaagtctgcaattggctaaggtggccatcgaagcaggcatggggctagtggaaattacctggaacagtgaccagccgggggaaaccattgctcacctgcgtcatcaatttccccattgtgccattggtacgggtacagtcctctctcaggaggatttgctccaggccgttgccagtggagcccaattttgctttaccccccacagtgagccagaattaattaccagcgccgtggcccatggcatccccattattcccggtgccttttcccctacggagattgtccaagcctggcagaggggagccaccgccgtcaaagtttttcccattaaaaccttgggaggagtggactacattcaagctctccaaggtcccctcggccaaattccccttattcccaccggaggggtaactttggcaaatgcccaagctttcctagcggccggagcgatcgccgttggcctatctggacagctattcccccccaatttagtagcaatcaaagcctggagagaaattggcgatctaatccgtactttgttgaaagaaatccagacaccaaggggctgaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 154 - DNA, 15242 bp, Artificial Sequence
  Synthetic construct plasmid pED03
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggatccaaaatgctgctggtggcggcggtgctaagcgccatgagcgccggaagtctcgccttcatgcccggacacacgggaaatgctttcgtctcgcaagcatctcagacctcctcttccttctcatcgccctccgtgcttccaagagccctcggagccaggggaagggctagggtcagcgccgcgggcctgtctatgcagatcaacaagtacagcaggaccctgacgcagaagaagtcacaaggagcgtcgcaggcgatgttgtatgctaccggtctgaagccggaggacatgaacaaagctcaggtgggtatttcaagtgtttggtatgagggaaatccttgcaatatgcacctcctcgaccttgcgggagaggtcaagaagggagttgaggcagctggcatggttggctaccgcttcaacaccatcggagtgtccgacggtatctcgatgggaaccgacggcatgtcatactcgcttcagtcacgagacatcattgcggactccattgagactgttgtctgtggacagtggtacgacggcaacatcgcacttcccggctgtgacaagaacatgccaggaaccattatcgccatggcgcgccacaaccgcccatccatcatgatctatggtggaactatcagggcaggaagcgccccggagatcgataggccagtgctcgacatcgtttcagctttccagtcttatggagagttccagtatggaaagatcactgaggaacagagacagaccattgtgcagaagagttgccctggacctggtgcttgcggtggcatgtacactgccaacaccatggcgacctcgatcgaggccctcggaatgtcgctcccctactccagctcttacccagctgactccaaggagaagaagatggagtgctacaactccggcaaggccatgatgaagctgctcgaaatggacctgaaaccgcgcgatatcatgaccagggaggccttcgagaacgccatggtcatggtcatggcgacgggcggcagcaccaacgctgtgctccatctcatcgccatggcgcgatctgtcggcatcaagctcactgtcgacgacttccagcgcgtcagcgacagagtgcccatgctcgccgacatgaagccctcgggcaagtacgtgatggaggacatccagaacatcggagggacgcctgcagtgatgaagtacctcgtggacaagaatctcttcgatgacagccagatgaccgtcaccggcaagacgatccgcgagaacctcgagagcctccccgggctcactgacggtcagaagatcatcctgccggtcgaggccccgatcaagaagaccgggcacttgcagatcctctacggtaacgtcgctcccgagggctccgtggccaagatcacgggcaaggaaggagagactttctctggaactgctgctgtgttcgactctgaggaggacatgctcgcagcgttggaccgcaaggagatcaaggcagggatggttgtggtgatccgctacgagggccccaagggaggccctggaatgccagagatgttgacgccgacgtcggcgatcatgggggccgggttagggaacgacgtggctctgatcactgacgggaggttctccggaggaacccacgggttctgcatcggtcacgtgacccccgaggcccaggttggcgggccgatcgccctgatccgcaatggcgacccgatccagatcgacgcgaggtcgagcgtgaggacgctgaacgtactggtgagcgacgaggagatggcgaagaggaaggcggaatggaaagctcctcctctcaaggccacccaagggactctgtacaagtacatcaaggcggtgtcgactgcgtccgaaggatgcgtgactgatgcttgacctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaattaattaatacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcagatctaaaatggctcaccgacttgctctagcgtcagctgcccgcctccgcgtatcccacgcgcgtctcttttcgagcgcggcttcatcgggtctcgatcgcgttcaaaccgtcgaaaactacgaacacgtcctgaacacctttgcgcaggtccgtgcttgcgccgttctccggaccgctacttcggatgattgccccaaggccatgcaagccgccattgacggtggtttccagatctgcgagttcaccctcacgactcccaactgtctgcagcatctggccgatttccgtgccaagtacgatggcaaggtcatgattggatgcgggaccattctgacgatcgatgatgctgccgctgctgtcgacgcaggcagtgagtttatcatcactcccgtcatgcttcccgacgttattgagtggtgcaaagagcgcaacattgtctccgtgcccggatgccaaacgcccacggaactcgtcaacgcgtatcgtcacggtgctcccttgcaaaagctattccccggtgtcgcgggtggacccggttgggtcaaagccgtctcgtcggcgttgccctttttgagcatcaaccccacgtctggggtggacctcgacaacgccggggagttccttaagaacggagccgcctccgtgggactcgtcgcacccctctttgatcccgtcgccgtcgcgaataaggactgggaccagattgctcagaacgccgccaaggtcatggccaatgtgcgtgaagtcggaccctaccagcgaaagaagtaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 155 - DNA, 15101 bp, Artificial Sequence
  Synthetic construct plasmid pED04
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggatccaaaatgcagtccctggcgctcacacccccctctctccccgcctccggccccacccccgcctccggccgtcgcctcgccacgcgtgtccgcgccgcctcggccgtctccgacgagcccaagctcaacaagtacagcgcgcgcatcacggagcccaagtcgcagggggcgtcgcaggccgtgctctacggcgtcggcctcaccgacgccgacctccgcaagccgcaggtcggggtctcctccgtctggtatgaggggaacacctgcaacatgcacctgctccagctcgccgaggccgtgcgcgacggaatccgcgacgccggcatggtcgccttccgcttcaacaccgtcggggtcagcgacgccatatccatgggcaccaggggcatgtgctacagcctccagtcgcgcgacctcatcgccgacagcatagagaccgtcatgggcgcgcagcactacgacgccaacatctcaatccctgggtgcgataagaacatgccaggtacaataatggcaatgggacggctaaatcgaccaagtatcatggtttatggtggaactattaagcctgggcactttcagggcaattcgtatgacatagtatctgcattccagagctacggagaatttgttagtgggtcaatcagcgatgaggaaagaaagaatgtactccgcaactcatgcccaggagcaggtgcttgtggcggtatgtacacagcaaacacaatggcatctgccatcgagacaatgggcatgagccttccatacagctcttcaacacctgctgaagactcattaaagctagatgaatgccgtcttgctgggaagtatctcttagaattgttaaagatggatttgaagcctagagacattataactgagaagtcattgcggaacgcaatggttattatcatggcacttggtgggtctactaatgctgtactgcatttgattgctattgccaggtctgtgggtttgcaattaactcttgatgatttccagaaggtcagcgaccaagttccttttcttgcagatcttaagcctagcggaaagtatgtcatggaggatctgcataagattggtggaacacctgcagtaattcattatctcttggagcaaggtcttcttgatggggattgtatgactgtcactggaaaaactctagcggaaaatgctaaaatattcccacctttatcggaaggacagcaaataatacgacggctcgacaatcctatcaaatcaactgggcatatacagatactttatggcaatcttgcaccagaaggctctgtagcaaaaataactggcaaggagggactgtttttctcaggccctgcactagtttttgaaggtgaagaatcaatgattacagctatatcagaaaacccagcaaatttcaagggaaaggttgtagtgatccgaggagaaggaccaaaaggaggtccagggatgcctgaaatgttgactccaacaagtgcaataatgggggctggtcttgggaaggagtgtgccctgctgacagatggtagattttctggggggtcgcatggatttgttgtgggccacgtatgtcctgaagcacaggaaggaggcccaattggtcttgttgaggacggtgatacaatcacgatcgacgtcgtgaagaaagtaattgatgttgatctgacggaagaccagctcgaacaaaggcgaaggaaatggagcccgccgccacacaaggttaccagtggagcactttggaagtacataaagcttgtgtcttcagcctcaagcgggtgcgtcaccgacgagtagcctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaattaattaatacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcagatctaaaatgctcatgctcgtcgccgcagcacggctgcctcttctcgcctcccagcctcgccggcgagatccgccgcggccaccgaaggcgctcgccgccatactgcgctcgcgcgtcattgcctgcctccgcgcgcaagacggcgagacggcgatgcaggcggcccacgcggcagttcgcggcggcgtctccgtgctagaaatcgtaatgtccactcctggagtgctagaggttgttgaggatctttgtaggagttatccttccttgacatttggggttggtacggtcctcaatgctgatgatgcaaggaaagccattagagctggtgcacagtttctcatgagccctggtacagtcatggaaatactttatgatcttgatggaagtgaaattctatatattccaggagtgctgacaccaactgaggttatatctgcttacaatgctggtgctaacgtcgttaaggtgtacccagtttcagtgatgggtggagagatgtacatgtctgctctaaagaaaccatttcctcttgtaccaatggttgcttctcaaggaattaccataggttcaatcaagtcgtacatggaggcaggggcatctgcagtggtgctgtccgatgctatctttggcaaagagttgatgagagaaagggatttcataggaatttcagcgcttgcaaatcaggccactttacaggcatcacagcgtggaagatgaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 156 - DNA, 17381 bp, Artificial Sequence
  Synthetic construct plasmid pED05
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcggatccaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgactgatctgcattcaacggtagaaaaggttaccgcgcgcgttattgaacgctcgcgggaaacccgtaaggcttatctggatttgatccagtatgagcgggaaaaaggcgtagaccgtccaaacctgtcctgtagtaaccttgctcatggctttgcggctatgaatggtgacaagccagctttgcgcgacttcaaccgcatgaatatcggcgtcgtgacttcctacaacgatatgttgtcggctcatgaaccatattatcgctatccggagcagatgaaagtatttgctcgcgaagttggcgcaacggttcaggtcgccggtggcgtgcctgctatgtgcgatggtgtgacccaaggtcagccgggcatggaagaatccctgtttagccgcgatgttatcgctttggctaccagcgtttctttgtctcatggtatgtttgaaggggctgcccttctcggtatctgtgacaagattgtccctggtctgttgatgggcgctctgcgctttggtcacctgccgaccattctggtcccatcaggcccgatgacgactggtatcccgaacaaagaaaaaatccgtatccgtcagctctatgctcagggtaaaatcggccagaaagaacttctggatatggaagcggcttgctaccatgctgaaggtacctgcaccttctatggtacggcaaacaccaaccagatggttatggaagtcctcggtcttcatatgccaggttcggcatttgttaccccgggtaccccgctccgccaggctctgacccgtgctgctgtgcatcgcgttgctgaattgggttggaagggcgacgattatcgtccgcttggtaaaatcattgacgaaaaatcaatcgtcaatgctattgttggtctgttggcaaccggtggttccaccaaccataccatgcatattccggccattgctcgtgctgctggtgttatcgttaactggaatgacttccatgatctttctgaagttgttccgttgattgcccgcatttacccgaatggcccgcgcgacatcaatgaattccagaatgcaggcggcatggcttatgtcatcaaagaactgctttctgctaatctgttgaaccgtgatgtcacgaccattgccaagggcggtatcgaagaatacgccaaggctccggcattaaatgatgctggcgaattggtctggaagccagctggcgaacctggtgatgacaccattctgcgtccggtttctaatcctttcgcaaaagatggcggtctgcgtctcttggaaggtaaccttggccgtgcaatgtacaaggccagtgcggttgatcctaaattctggaccattgaagcaccggttcgcgtcttctctgaccaagacgatgttcagaaagccttcaaggctggcgaattgaacaaagacgttatcgttgttgttcgtttccagggcccgcgcgcaaacggtatgcctgaattgcataagctgaccccggctttgggtgttctgcaggataatggctacaaagttgctttggtaactgatggtcgtatgtccggtgctaccggtaaagttccggttgctttgcatgtcagcccagaagctcttggcggtggtgccatcggtaaattacgtgatggcgatatcgtccgtatctcggttgaagaaggcaaacttgaagctttggttccagctgatgagtggaatgctcgtccgcatgctgaaaaaccggctttccgtccgggaaccggacgcgaattgtttgatatcttccgtcagaatgctgctaaagctgaagacggtgcagtcgcaatatatgcaggtgccggtatctaacctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacggaccgctcgagcaattgtacgtagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcttcgaaaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgcgtgatatcgattccgtaatgcgtttggcaccggttatgccggtcctcgtcattgaagatattgctgatgcaaaacctatcgcagaagctttggttgctggtggtctgaacgttcttgaagtaacgcttcgcaccccttgtgctcttgaagccatcaagatcatgaaagaagttccgggtgccgttgttggtgccggtacggttctgaacgcaaaaatgctcgaccaagctcaggaagctggttgcgaatttttcgttagcccgggtctgaccgctgacctcggcaagcatgctgttgcccagaaagcagctttgcttccaggtgttgctaatgctgctgatgtgatgcttggtcttgaccttggtcttgatcgcttcaaattcttcccggctgaaaatatcggtggtttacctgccctgaagtccatggcttctgttttccgtcaggttcgtttctgcccgaccggcggtatcaccccgacgtcagctcctaaatatcttgaaaacccgtccattctttgcgtcggtggtagctgggttgttccggctggcaaaccagatgtcgcaaaaatcacggcactcgctaaagaagcttctgctttcaagcgcgctgctgttgcctaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 157 - DNA, 17237 bp, Artificial Sequence
  Synthetic construct plasmid pED06
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcggatccaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgtccaacaatcccagaagccaggtaattacccagggcacccaacggagtcccaaccgggccatgctccgggccgttggttttggcgatgatgactttacgaaacccattgtgggtattgccaacgggtacagcaccattaccccctgcaacatgggcattaatgatctggctctacgggccgaggccggcttacggacagcgggggccatgccccaattgtttggcactatcaccatcagtgacggtatttccatgggtacggaagggatgaaatattccctagtttcccgggaagtgattgctgattccatcgaaacagtatgcaatggccaacgcatggacggagttttggcgatcgggggctgtgataagaatatgccgggggcaatgattgccatggctcgcctcaatattccctctatctttgtttacggtggcactattaaacccggacattatgccggggaagatttaacagtggtcagtgcctttgaagcggtgggccagtacagcgctggcaaaattgatgaggaaaccctctacggcattgaacggaatgcctgtcctggggctggttcctgtggggggatgttcaccgccaacaccatgtcctccgcctttgaagctatggggatgagcttgccctattcttctaccatggccgcagtggatggggaaaaagccgacagcaccgaggagtccgccaaggttttggtggaagccatcaaaaaacagattttgcccagtcaaattctgacccggaaagcttttgaaaatgccattgccgtgattatggccgtggggggctccactaatgcagtgctccatctgctggcgatcgccaacaccattggggtgcccctaagcttggacgattttgaaaccattcgccacaaagtacctgtactgtgcgacctcaaaccttccggcaaatacgtcaccactaatctccacgccgccggtggcattccccaggtgatgaaaatattgctggtcaatggcattctccacggtgacgctttaacgattacaggacagaccattgctgaagttttagccgatattcctgaccaaccccctgccggccaggacgtaatccattcctgggatgaccctgtttatcaagaaggtcacctggcagtgctcaaaggtaacctggccaccgaaggcagtgtggctaaaatcagcggtgttaaaaagccggtgatcactggtcccgccaaagtatttgaatctgaagaagattgcctagaagcgattctagccggcaaaattcaagcaggagatgtggtggtcgtgcgctatgaagggcccaaaggtggccccggtatgcgggaaatgctagcccccacttctgccatcattggggcagggttgggagactccgttggcctaatcaccgatggccgcttttctggtggtacctatggcttagtggtgggtcacgttgcccccgaagcttatgtgggaggggcgatcgccttggtgcaagagggggatcaaatcaccatcgatgctggcaagcggttactgcaactcaacatcagtgaagaggaattggcccaacggcgggcccaatggactcctcctcagccccgctatccccgggggattttagccaagtatgccaagttagtttcctccagcagccttggtgcagtgactgatatagacttgttctaacctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacggaccgctcgagcaattgtacgtagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcttcgaaaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgttgcgacagcaccgggcgatcgccgttattcgcactgattccatcgacctaagtctgcaattggctaaggtggccatcgaagcaggcatggggctagtggaaattacctggaacagtgaccagccgggggaaaccattgctcacctgcgtcatcaatttccccattgtgccattggtacgggtacagtcctctctcaggaggatttgctccaggccgttgccagtggagcccaattttgctttaccccccacagtgagccagaattaattaccagcgccgtggcccatggcatccccattattcccggtgccttttcccctacggagattgtccaagcctggcagaggggagccaccgccgtcaaagtttttcccattaaaaccttgggaggagtggactacattcaagctctccaaggtcccctcggccaaattccccttattcccaccggaggggtaactttggcaaatgcccaagctttcctagcggccggagcgatcgccgttggcctatctggacagctattcccccccaatttagtagcaatcaaagcctggagagaaattggcgatctaatccgtactttgttgaaagaaatccagacaccaaggggctgaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 158 - DNA, 17072 bp, Artificial Sequence
  Synthetic construct plasmid pED07
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcggatccaaaatgctgctggtggcggcggtgctaagcgccatgagcgccggaagtctcgccttcatgcccggacacacgggaaatgctttcgtctcgcaagcatctcagacctcctcttccttctcatcgccctccgtgcttccaagagccctcggagccaggggaagggctagggtcagcgccgcgggcctgtctatgcagatcaacaagtacagcaggaccctgacgcagaagaagtcacaaggagcgtcgcaggcgatgttgtatgctaccggtctgaagccggaggacatgaacaaagctcaggtgggtatttcaagtgtttggtatgagggaaatccttgcaatatgcacctcctcgaccttgcgggagaggtcaagaagggagttgaggcagctggcatggttggctaccgcttcaacaccatcggagtgtccgacggtatctcgatgggaaccgacggcatgtcatactcgcttcagtcacgagacatcattgcggactccattgagactgttgtctgtggacagtggtacgacggcaacatcgcacttcccggctgtgacaagaacatgccaggaaccattatcgccatggcgcgccacaaccgcccatccatcatgatctatggtggaactatcagggcaggaagcgccccggagatcgataggccagtgctcgacatcgtttcagctttccagtcttatggagagttccagtatggaaagatcactgaggaacagagacagaccattgtgcagaagagttgccctggacctggtgcttgcggtggcatgtacactgccaacaccatggcgacctcgatcgaggccctcggaatgtcgctcccctactccagctcttacccagctgactccaaggagaagaagatggagtgctacaactccggcaaggccatgatgaagctgctcgaaatggacctgaaaccgcgcgatatcatgaccagggaggccttcgagaacgccatggtcatggtcatggcgacgggcggcagcaccaacgctgtgctccatctcatcgccatggcgcgatctgtcggcatcaagctcactgtcgacgacttccagcgcgtcagcgacagagtgcccatgctcgccgacatgaagccctcgggcaagtacgtgatggaggacatccagaacatcggagggacgcctgcagtgatgaagtacctcgtggacaagaatctcttcgatgacagccagatgaccgtcaccggcaagacgatccgcgagaacctcgagagcctccccgggctcactgacggtcagaagatcatcctgccggtcgaggccccgatcaagaagaccgggcacttgcagatcctctacggtaacgtcgctcccgagggctccgtggccaagatcacgggcaaggaaggagagactttctctggaactgctgctgtgttcgactctgaggaggacatgctcgcagcgttggaccgcaaggagatcaaggcagggatggttgtggtgatccgctacgagggccccaagggaggccctggaatgccagagatgttgacgccgacgtcggcgatcatgggggccgggttagggaacgacgtggctctgatcactgacgggaggttctccggaggaacccacgggttctgcatcggtcacgtgacccccgaggcccaggttggcgggccgatcgccctgatccgcaatggcgacccgatccagatcgacgcgaggtcgagcgtgaggacgctgaacgtactggtgagcgacgaggagatggcgaagaggaaggcggaatggaaagctcctcctctcaaggccacccaagggactctgtacaagtacatcaaggcggtgtcgactgcgtccgaaggatgcgtgactgatgcttgacctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacggaccgctcgagcaattgtacgtagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcttcgaaaaaatggctcaccgacttgctctagcgtcagctgcccgcctccgcgtatcccacgcgcgtctcttttcgagcgcggcttcatcgggtctcgatcgcgttcaaaccgtcgaaaactacgaacacgtcctgaacacctttgcgcaggtccgtgcttgcgccgttctccggaccgctacttcggatgattgccccaaggccatgcaagccgccattgacggtggtttccagatctgcgagttcaccctcacgactcccaactgtctgcagcatctggccgatttccgtgccaagtacgatggcaaggtcatgattggatgcgggaccattctgacgatcgatgatgctgccgctgctgtcgacgcaggcagtgagtttatcatcactcccgtcatgcttcccgacgttattgagtggtgcaaagagcgcaacattgtctccgtgcccggatgccaaacgcccacggaactcgtcaacgcgtatcgtcacggtgctcccttgcaaaagctattccccggtgtcgcgggtggacccggttgggtcaaagccgtctcgtcggcgttgccctttttgagcatcaaccccacgtctggggtggacctcgacaacgccggggagttccttaagaacggagccgcctccgtgggactcgtcgcacccctctttgatcccgtcgccgtcgcgaataaggactgggaccagattgctcagaacgccgccaaggtcatggccaatgtgcgtgaagtcggaccctaccagcgaaagaagtaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 159 - DNA, 16931 bp, Artificial Sequence
  Synthetic construct plasmid pED08
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcggatccaaaatgcagtccctggcgctcacacccccctctctccccgcctccggccccacccccgcctccggccgtcgcctcgccacgcgtgtccgcgccgcctcggccgtctccgacgagcccaagctcaacaagtacagcgcgcgcatcacggagcccaagtcgcagggggcgtcgcaggccgtgctctacggcgtcggcctcaccgacgccgacctccgcaagccgcaggtcggggtctcctccgtctggtatgaggggaacacctgcaacatgcacctgctccagctcgccgaggccgtgcgcgacggaatccgcgacgccggcatggtcgccttccgcttcaacaccgtcggggtcagcgacgccatatccatgggcaccaggggcatgtgctacagcctccagtcgcgcgacctcatcgccgacagcatagagaccgtcatgggcgcgcagcactacgacgccaacatctcaatccctgggtgcgataagaacatgccaggtacaataatggcaatgggacggctaaatcgaccaagtatcatggtttatggtggaactattaagcctgggcactttcagggcaattcgtatgacatagtatctgcattccagagctacggagaatttgttagtgggtcaatcagcgatgaggaaagaaagaatgtactccgcaactcatgcccaggagcaggtgcttgtggcggtatgtacacagcaaacacaatggcatctgccatcgagacaatgggcatgagccttccatacagctcttcaacacctgctgaagactcattaaagctagatgaatgccgtcttgctgggaagtatctcttagaattgttaaagatggatttgaagcctagagacattataactgagaagtcattgcggaacgcaatggttattatcatggcacttggtgggtctactaatgctgtactgcatttgattgctattgccaggtctgtgggtttgcaattaactcttgatgatttccagaaggtcagcgaccaagttccttttcttgcagatcttaagcctagcggaaagtatgtcatggaggatctgcataagattggtggaacacctgcagtaattcattatctcttggagcaaggtcttcttgatggggattgtatgactgtcactggaaaaactctagcggaaaatgctaaaatattcccacctttatcggaaggacagcaaataatacgacggctcgacaatcctatcaaatcaactgggcatatacagatactttatggcaatcttgcaccagaaggctctgtagcaaaaataactggcaaggagggactgtttttctcaggccctgcactagtttttgaaggtgaagaatcaatgattacagctatatcagaaaacccagcaaatttcaagggaaaggttgtagtgatccgaggagaaggaccaaaaggaggtccagggatgcctgaaatgttgactccaacaagtgcaataatgggggctggtcttgggaaggagtgtgccctgctgacagatggtagattttctggggggtcgcatggatttgttgtgggccacgtatgtcctgaagcacaggaaggaggcccaattggtcttgttgaggacggtgatacaatcacgatcgacgtcgtgaagaaagtaattgatgttgatctgacggaagaccagctcgaacaaaggcgaaggaaatggagcccgccgccacacaaggttaccagtggagcactttggaagtacataaagcttgtgtcttcagcctcaagcgggtgcgtcaccgacgagtagcctaagggcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacggaccgctcgagcaattgtacgtagaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcttcgaaaaaatgctcatgctcgtcgccgcagcacggctgcctcttctcgcctcccagcctcgccggcgagatccgccgcggccaccgaaggcgctcgccgccatactgcgctcgcgcgtcattgcctgcctccgcgcgcaagacggcgagacggcgatgcaggcggcccacgcggcagttcgcggcggcgtctccgtgctagaaatcgtaatgtccactcctggagtgctagaggttgttgaggatctttgtaggagttatccttccttgacatttggggttggtacggtcctcaatgctgatgatgcaaggaaagccattagagctggtgcacagtttctcatgagccctggtacagtcatggaaatactttatgatcttgatggaagtgaaattctatatattccaggagtgctgacaccaactgaggttatatctgcttacaatgctggtgctaacgtcgttaaggtgtacccagtttcagtgatgggtggagagatgtacatgtctgctctaaagaaaccatttcctcttgtaccaatggttgcttctcaaggaattaccataggttcaatcaagtcgtacatggaggcaggggcatctgcagtggtgctgtccgatgctatctttggcaaagagttgatgagagaaagggatttcataggaatttcagcgcttgcaaatcaggccactttacaggcatcacagcgtggaagatgaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 160 - DNA, 2073 bp, Artificial Sequence
  Synthetic construct pea plastid targeting signal fused to Zymomonas mobilis EDD gene
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgactgatctgcattcaacggtagaaaaggttaccgcgcgcgttattgaacgctcgcgggaaacccgtaaggcttatctggatttgatccagtatgagcgggaaaaaggcgtagaccgtccaaacctgtcctgtagtaaccttgctcatggctttgcggctatgaatggtgacaagccagctttgcgcgacttcaaccgcatgaatatcggcgtcgtgacttcctacaacgatatgttgtcggctcatgaaccatattatcgctatccggagcagatgaaagtatttgctcgcgaagttggcgcaacggttcaggtcgccggtggcgtgcctgctatgtgcgatggtgtgacccaaggtcagccgggcatggaagaatccctgtttagccgcgatgttatcgctttggctaccagcgtttctttgtctcatggtatgtttgaaggggctgcccttctcggtatctgtgacaagattgtccctggtctgttgatgggcgctctgcgctttggtcacctgccgaccattctggtcccatcaggcccgatgacgactggtatcccgaacaaagaaaaaatccgtatccgtcagctctatgctcagggtaaaatcggccagaaagaacttctggatatggaagcggcttgctaccatgctgaaggtacctgcaccttctatggtacggcaaacaccaaccagatggttatggaagtcctcggtcttcatatgccaggttcggcatttgttaccccgggtaccccgctccgccaggctctgacccgtgctgctgtgcatcgcgttgctgaattgggttggaagggcgacgattatcgtccgcttggtaaaatcattgacgaaaaatcaatcgtcaatgctattgttggtctgttggcaaccggtggttccaccaaccataccatgcatattccggccattgctcgtgctgctggtgttatcgttaactggaatgacttccatgatctttctgaagttgttccgttgattgcccgcatttacccgaatggcccgcgcgacatcaatgaattccagaatgcaggcggcatggcttatgtcatcaaagaactgctttctgctaatctgttgaaccgtgatgtcacgaccattgccaagggcggtatcgaagaatacgccaaggctccggcattaaatgatgctggcgaattggtctggaagccagctggcgaacctggtgatgacaccattctgcgtccggtttctaatcctttcgcaaaagatggcggtctgcgtctcttggaaggtaaccttggccgtgcaatgtacaaggccagtgcggttgatcctaaattctggaccattgaagcaccggttcgcgtcttctctgaccaagacgatgttcagaaagccttcaaggctggcgaattgaacaaagacgttatcgttgttgttcgtttccagggcccgcgcgcaaacggtatgcctgaattgcataagctgaccccggctttgggtgttctgcaggataatggctacaaagttgctttggtaactgatggtcgtatgtccggtgctaccggtaaagttccggttgctttgcatgtcagcccagaagctcttggcggtggtgccatcggtaaattacgtgatggcgatatcgtccgtatctcggttgaagaaggcaaacttgaagctttggttccagctgatgagtggaatgctcgtccgcatgctgaaaaaccggctttccgtccgggaaccggacgcgaattgtttgatatcttccgtcagaatgctgctaaagctgaagacggtgcagtcgcaatatatgcaggtgccggtatctaa

SEQ ID NO: 161 - DNA, 876 bp, Artificial Sequence
  Synthetic construct pea plastid targeting signal fused to Zymomonas mobilis EDA gene
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgcgtgatatcgattccgtaatgcgtttggcaccggttatgccggtcctcgtcattgaagatattgctgatgcaaaacctatcgcagaagctttggttgctggtggtctgaacgttcttgaagtaacgcttcgcaccccttgtgctcttgaagccatcaagatcatgaaagaagttccgggtgccgttgttggtgccggtacggttctgaacgcaaaaatgctcgaccaagctcaggaagctggttgcgaatttttcgttagcccgggtctgaccgctgacctcggcaagcatgctgttgcccagaaagcagctttgcttccaggtgttgctaatgctgctgatgtgatgcttggtcttgaccttggtcttgatcgcttcaaattcttcccggctgaaaatatcggtggtttacctgccctgaagtccatggcttctgttttccgtcaggttcgtttctgcccgaccggcggtatcaccccgacgtcagctcctaaatatcttgaaaacccgtccattctttgcgtcggtggtagctgggttgttccggctggcaaaccagatgtcgcaaaaatcacggcactcgctaaagaagcttctgctttcaagcgcgctgctgttgcctaa

SEQ ID NO: 162 - DNA, 1707 bp, Artificial Sequence
  Synthetic construct pea plastid targeting signal fused to Zymomonas mobilis ZWF gene
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgacaaataccgtttcgacgatgatattgtttggctcgactggcgacctttcacagcgtatgctgttgccgtcgctttatggtcttgatgccgatggtttgcttgcagatgatctgcgtatcgtctgcacctctcgtagcgaatacgacacagatggtttccgtgattttgcagaaaaagctttagatcgctttgtcgcttctgaccggttaaatgatgacgctaaagctaaattccttaacaagcttttctacgcgacggtcgatattacggatccgacccaattcggaaaattagctgacctttgtggcccggtcgaaaaaggtatcgccatttatctttcgactgcgccttctttgtttgaaggggcaatcgctggcctgaaacaggctggtctggctggtccaacttctcgcctggcgcttgaaaaacctttaggtcaagatcttgcttcttccgatcatattaatgatgcggttttgaaagttttctctgaaaagcaagtttatcgtattgaccattatctgggtaaagaaacggttcagaatcttctgaccctgcgttttggtaatgctttgtttgaaccgctttggaattcaaaaggcattgaccacgttcagatcagcgttgctgaaacggttggtcttgaaggtcgtatcggttatttcgacggttctggcagcttgcgcgatatggttcaaagccatatccttcagttggtcgctttggttgcaatggaaccaccggctcatatggaagccaacgctgttcgtgacgaaaaggtaaaagttttccgcgctctgcgtccgatcaataacgacaccgtctttacgcataccgttaccggtcaatatggtgccggtgtttctggtggtaaagaagttgccggttacattgacgaactgggtcagccttccgataccgaaacctttgttgctatcaaagcgcatgttgataactggcgttggcagggtgttccgttctatatccgcactggtaagcgtttacctgcacgtcgttctgaaatcgtggttcagtttaaacctgttccgcattcgattttctcttcttcaggtggtatcttgcagccgaacaagctgcgtattgtcttacagcctgatgaaaccatccagatttctatgatggtgaaagaaccgggtcttgaccgtaacggtgcgcatatgcgtgaagtttggctggatctttccctcacggatgtgtttaaagaccgtaaacgtcgtatcgcttatgaacgcctgatgcttgatcttatcgaaggcgatgctactttatttgtgcgtcgtgacgaagttgaggcgcagtgggtttggattgacggaattcgtgaaggctggaaagccaacagtatgaagccaaaaacctatgtctctggtacatgggggccttcaactgctatagctctggccgaacgtgatggagtaacttggtatgactga

SEQ ID NO: 163 - DNA, 957 bp, Artificial Sequence
  Synthetic construct pea plastid targeting signal fused to Zymomonas mobilis PGL gene
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtgaccgaagccgaatggtgggaatttgaaaatgtcgaagctatggcaaaacagattgccgacgacatcgaatttattatcaagcaagccattgagaaaaaaggccgcgctttaatcatcgttccgggtggttctacaccgaaactggttttcccgacgctcgcggctcgtgacctcgattggtcaaaagtaacgctgatgctgaccgatgaccgtctggttgccaaagataatccgttaagcaattttggccttctgaccaaacatttcggcagtagcggtgctgaactggtctccttgattgatgaaaattatctggatgaccgcgctgccgctggccgtgctgcagaccagaaactggcttcttataaatggcctgccgatctcgtttggttgggtatgggtaatgatggccataccgcttctattttcccaggtcccaatttcgacgaagctgttaacgggccccgtgaacgccgcgctcttggccttcttccggttccgttaccgccagaagcaccggttgcccgcgtaacccttagcttgtccacgctggcttctgctcatactgttatggtggttatcactggcgatcataaacgcacggttctgaccgatgctttgaaagaaggcgcttccagccgtcttccggttggccgcgttctcggtgaaaccgaagcctccatcgacgtgtactggagcaaataa

SEQ ID NO: 164 - DNA, 504 bp, Glycine max
tgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaa